(19) United States
(12) Patent Application Publication
(10) Pub. No.: US 2014/0295512 A1
(43) Pub. Date: Oct. 2, 2014

(54) Ketol-Acid Reductoisomerases With Improved Performance Properties

(71) Applicants: Thomas Buelter, Duisburg (DE); Doug Lies, Parker, CO (US); Stephanie Porter-Scheinman, Conifer, CO (US); Christopher Smith, Parker, CO (US); Peter Meinhold, Topanga, CA (US); Ruth Berry, Bellevne, WA (US); Christopher Snow, Fort Collins, CO (US); Sabine Bastian, Pasadena, CA (US)

(72) Inventors: Thomas Buelter, Duisburg (DE); Doug Lies, Parker, CO (US); Stephanie Porter-Scheinman, Conifer, CO (US); Christopher Smith, Parker, CO (US); Peter Meinhold, Topanga, CA (US); Ruth Berry, Bellevne, WA (US); Christopher Snow, Fort Collins, CO (US); Sabine Bastian, Pasadena, CA (US)

(73) Assignees: CALIFORNIA INSTITUTE OF TECHNOLOGY, Pasadena, CA (US); GEVO, INC., Englewood, CO (US)

(21) Appl. No.: 14/126,606

(22) PCT Filed: Jun. 15, 2012
(86) PCT No.: PCT/US12/42624

§ 371 Date: Jun. 16, 2014

Related U.S. Application Data
(60) Provisional application No. 61/497,851, filed on Jun. 16, 2011.
(60) Provisional application No. 61/510,617, filed on Jul. 22, 2011.

(51) Int. Cl. C12P 7/16; C12N 9/04

(52) U.S. Cl. CPC C12P 7/16; C12N 9/0006
USPC 435/160; 435/254.2; 435/190

(57) ABSTRACT

The present invention relates to recombinant microorganisms comprising at least one nucleic acid molecule encoding a ketol-acid reductoisomerase (KARI) or modified NADH-dependent variant thereof, wherein said KARI is at least about 60% identical to SEQ ID NO: 2. In various aspects of the invention, the recombinant microorganisms may comprise an isobutanol producing metabolic pathway and can be used in methods of making isobutanol.

### CROSS-REFERENCE TO RELATED APPLICATIONS

This application is a U.S. national stage of International Application No. PCT/US2012/042624, filed Jun. 15, 2012, which claims priority to U.S. Provisional Application Ser. No. 61/497,851, filed Jun. 16, 2011, and U.S. Provisional Application Ser. No. 61/510,617, filed Jul. 22, 2011, each of which is herein incorporated by reference in their entireties for all purposes.

### ACKNOWLEDGMENT OF GOVERNMENTAL SUPPORT

This invention was made with government support under Contract No. 2009-10006-05919, awarded by the United States Department of Agriculture, and under Contract No. W911NF-09-2-0022, awarded by the United States Army Research Laboratory. The government has certain rights in the invention.

### TECHNICAL FIELD

Recombinant microorganisms and methods of producing such microorganisms are provided. Also provided are methods of producing beneficial metabolites including fuels and chemicals by contacting a suitable substrate with the recombinant microorganisms of the invention and enzymatic preparations therefrom.

### DESCRIPTION OF THE TEXT FILE SUBMITTED ELECTRONICALLY

The contents of the text file submitted electronically herewith are incorporated herein by reference in their entirety: A computer readable format copy of the Sequence Listing (filename: GEVO_{—}063_{—}02US_SeqList_ST25.txt, date recorded: Dec. 16, 2013, file size: 49 kilobytes).

### BACKGROUND

The ability of microorganisms to convert sugars to beneficial metabolites including fuels, chemicals, and amino acids has been widely described in the literature in recent years. See, e.g., Alper et al., 2009, Nature Microbiol. Rev. 7: 715-723 and McCourt et al., 2006, Amino Acids 31: 173-210. Recombinant engineering techniques have enabled the creation of microorganisms that express biosynthetic pathways capable of producing a number of useful products, including the commodity chemical, isobutanol.

Isobutanol, also a promising biofuel candidate, has been produced in recombinant microorganisms expressing a heterologous, five-step metabolic pathway (See, e.g., WO/2007/050671 to Donaldson et al., WO/2008/098227 to Liao et al., and WO/2009/103533 to Festel et al.). However, the microorganisms produced to date have fallen short of commercial relevance due to their low performance characteristics, including, for example low productivities, low titers, and low yields.

The second step of the isobutanol producing metabolic pathway is catalyzed by ketol-acid reductoisomerase (KARI), which converts acetolactate to 2,3-dihydroxyisovalerate. The present inventors have observed that KARI enzymes currently being used in isobutanol-producing recombinant microorganisms suffer from product inhibition (i.e., inhibition by 2,3-dihydroxyisovalerate), resulting in low isobutanol productivity. To overcome this problem and thereby improve isobutanol production, the present inventors have identified a group of KARI enzymes exhibiting reduced inhibition by 2,3-dihydroxyisovalerate. Accordingly, this application describes methods of increasing isobutanol production through the use of recombinant microorganisms comprising KARI enzymes with improved properties for the production of isobutanol.

One KARI enzyme of particular interest identified herein that exhibits reduced product inhibition is the S. exigua KARI enzyme (SEQ ID NO: 2). The present inventors have found that the use of the S. exigua KARI enzyme in an isobutanol pathway may lead to improved isobutanol yields, titers, and productivity.

Another important feature of a KARI enzyme is the ability to use NADH as a cofactor for the conversion of acetolactate to 2,3-dihydroxyisovalerate. The present inventors have found that when an NADH-dependent KARI is used in conjunction with an NADH-dependent alcohol dehydrogenase (ADH), isobutanol can be produced at theoretical yield and/or under anaerobic conditions. See, e.g., commonly owned and co-pending US Publication No. US 2010/0143997. Because NADH-dependence is an important feature of a KARI enzyme, the present inventors have identified several beneficial mutations which can be made to the S. exigua KARI enzyme to switch the cofactor specificity of the enzyme from NADPH to NADH.

### SUMMARY OF THE INVENTION

The present inventors have discovered that a group of KARI enzymes with reduced inhibition by 2,3-dihydroxyisovalerate. The use of one or more of these KARI enzymes, or NADH-dependent variants thereof, can improve production of the isobutanol.

In a first aspect, the application relates to a recombinant microorganism comprising at least one nucleic acid molecule encoding a ketol-acid reductoisomerase (KARI), wherein said KARI is at least about 60% identical to SEQ ID NO: 2 and/or SEQ ID NO: 12. In one embodiment, the KARI is derived from the genus Slackia. In a specific embodiment, the KARI is derived from Slackia exigua. In another specific embodiment, the KARI is encoded by SEQ ID NO: 1. In another embodiment, the KARI is derived from the genus Cryptobacterium. In a specific embodiment, the KARI is derived from Cryptobacterium curtum. In another specific embodiment, the KARI is encoded by SEQ ID NO: 3. In yet another embodiment, the KARI is derived from the genus Eggerthella. In a specific embodiment, the KARI is derived from Eggerthella lenta. In another specific embodiment, the KARI is encoded by SEQ ID NO: 5, SEQ ID NO: 7, or SEQ ID NO: 9.

In some embodiments, the KARI may be modified to be NADH-dependent. Accordingly, the present application further relates to NADH-dependent ketol-acid reductoisomerases (NKRs) derived from a KARI that is at least about 60% identical to SEQ ID NO: 2 and/or SEQ ID NO: 12. Thus, in one embodiment, the present application relates to a recombinant microorganism comprising a NKR derived from a KARI that is at least about 60% identical to SEQ ID NO: 2 and/or SEQ ID NO: 12.

Therefore, the present application also relates to mutated ketol-acid reductoisomerase (KARI) enzymes that utilize NADH rather than NADPH. Examples of such KARIs include enzymes having one or more modifications or mutations at positions corresponding to amino acids selected from: (a) tyrosine 35 of the S. exigua KARI (SEQ ID NO: 2); (b) leucine 57 of the S. exigua KARI (SEQ ID NO: 2); (c) arginine 58 of the S. exigua KARI (SEQ ID NO: 2); (d) glycine 60 of the S. exigua KARI (SEQ ID NO: 2); (e) serine 61 of the S. exigua KARI (SEQ ID NO: 2); (f) serine 62 of the S. exigua KARI (SEQ ID NO: 2); (g) serine 63 of the S. exigua KARI (SEQ ID NO: 2); (h) isoleucine 95 of the S. exigua KARI (SEQ ID NO: 2); and (i) valine 99 of the S. exigua KARI (SEQ ID NO: 2).

In one embodiment, the KARI enzyme contains a modification or mutation at the amino acid corresponding to position 35 of the S. exigua KARI (SEQ ID NO: 2). In another embodiment, the KARI enzyme contains a modification or mutation at the amino acid corresponding to position 57 of the S. exigua KARI (SEQ ID NO: 2). In yet another embodiment, the KARI enzyme contains a modification or mutation at the amino acid corresponding to position 58 of the S. exigua KARI (SEQ ID NO: 2). In yet another embodiment, the KARI enzyme contains a modification or mutation at the amino acid corresponding to position 60 of the S. exigua KARI (SEQ ID NO: 2). In yet another embodiment, the KARI enzyme contains a modification or mutation at the amino acid corresponding to position 61 of the S. exigua KARI (SEQ ID NO: 2). In yet another embodiment, the KARI enzyme contains a modification or mutation at the amino acid corresponding to position 62 of the S. exigua KARI (SEQ ID NO: 2). In yet another embodiment, the KARI enzyme contains a modification or mutation at the amino acid corresponding to position 63 of the S. exigua KARI (SEQ ID NO: 2). In yet another embodiment, the KARI enzyme contains a modification or mutation at the amino acid corresponding to position 95 of the S. exigua KARI (SEQ ID NO: 2). In yet another embodiment, the KARI enzyme contains a modification or mutation at the amino acid corresponding to position 99 of the S. exigua KARI (SEQ ID NO: 2).

In one embodiment, the KARI enzyme contains two or more modifications or mutations at the amino acids corresponding to the positions described above. In another embodiment, the KARI enzyme contains three or more modifications or mutations at the amino acids corresponding to the positions described above. In yet another embodiment, the KARI enzyme contains four or more modifications or mutations at the amino acids corresponding to the positions described above. In yet another embodiment, the KARI enzyme contains five or more modifications or mutations at the amino acids corresponding to the positions described above. In yet another embodiment, the KARI enzyme contains six or more modifications or mutations at the amino acids corresponding to the positions described above. In yet another embodiment, the KARI enzyme contains seven or more modifications or mutations at the amino acids corresponding to the positions described above. In yet another embodiment, the KARI enzyme contains eight modifications or mutations at the amino acids corresponding to the positions described above. In yet another embodiment, the KARI enzyme contains nine modifications or mutations at the amino acids corresponding to the positions described above.

Further included within the scope of the application are KARI enzymes, other than the S. exigua KARI (SEQ ID NO: 2), which contain modifications or mutations corresponding to those set out above.

In various embodiments described herein, the modified or mutated KARI may exhibit an increased catalytic efficiency with NADH as compared to the wild-type KARI. In one embodiment, the KARI has at least about a 5% increased catalytic efficiency with NADH as compared to the wild-type KARI. In another embodiment, the KARI has at least about a 25%, at least about a 50%, at least about a 75%, at least about a 100%, at least about a 500%, at least about 1000%, or at least about a 10000% increased catalytic efficiency with NADH as compared to the wild-type KARI.

In various embodiments described herein, the modified or mutated KARI may exhibit a decreased Michaelis Menten constant (K_{M}) for NADH as compared to the wild-type KARI. In one embodiment, the KARI has at least about a 5% decreased K_{M} for NADH as compared to the wild-type KARI. In another embodiment, the KARI has at least about a 25%, at least about a 50%, at least about a 75%, at least about a 90%, at least about a 95%, or at least about a 97.5% decreased K_{M} for NADH as compared to the wild-type KARI.

In various embodiments described herein, the modified or mutated KARI may exhibit an increased catalytic constant (k_{cat}) with NADH as compared to the wild-type KARI. In one embodiment, the KARI has at least about a 5% increased k_{cat} with NADH as compared to the wild-type KARI. In another embodiment, the KARI has at least about a 25%, at least about a 50%, at least about a 75%, at least about 100%, at least about 200%, or at least about a 500% increased k_{cat} with NADH as compared to the wild-type KARI.

In various embodiments described herein, the modified or mutated KARI may exhibit an increased Michaelis Menten constant (K_{M}) for NADPH as compared to the wild-type KARI. In one embodiment, the KARI has at least about a 5% increased K_{M} for NADPH as compared to the wild-type KARI. In another embodiment, the KARI has at least about a 25%, at least about a 50%, at least about a 100%, at least about a 500%, at least about a 1000%, or at least about a 5000% increased K_{M} for NADPH as compared to the wild-type KARI.

In various embodiments described herein, the modified or mutated KARI may exhibit a decreased catalytic constant (k_{cat}) with NADPH as compared to the wild-type KARI. In one embodiment, the KARI has at least about a 5% decreased k_{cat} with NADPH as compared to the wild-type KARI. In another embodiment, the KARI has at least about a 25%, at least about a 50%, or at least about a 75%, at least about 90% decreased k_{cat} with NADPH as compared to the wild-type KARI.

In some embodiments described herein, the catalytic efficiency of the modified or mutated KARI with NADH is increased with respect to the catalytic efficiency with NADPH of the wild-type KARI. In one embodiment, the catalytic efficiency of said KARI with NADH is at least about 10% of the catalytic efficiency with NADPH of the wild-type KARI. In another embodiment, the catalytic efficiency of said KARI with NADH is at least about 25%, at least about 50%, or at least about 75% of the catalytic efficiency with NADPH of the wild-type KARI. In some embodiments, the modified or mutated KARI preferentially utilizes NADH rather than NADPH.

In one embodiment, the application is directed to NADH-dependent KARI enzymes having a catalytic efficiency with NADH that is greater than the catalytic efficiency with NADPH. In one embodiment, the catalytic efficiency of the NADH-dependent KARI is at least about 2-fold greater with NADH than with NADPH. In another embodiment, the catalytic efficiency of the NADH-dependent KARI is at least about 4-fold, at least about 5-fold, at least about 6-fold, at least about 7-fold, at least about 8-fold, at least about 9-fold, at least about 10-fold, at least about 25-fold, at least about 50-fold, at least about 100-fold, or at least about 500-fold greater with NADH than with NADPH.

In one embodiment, the application is directed to modified or mutated KARI enzymes that demonstrate a switch in cofactor specificity from NADPH to NADH. In one embodiment, the modified or mutated KARI has at least about a 2:1 ratio of catalytic efficiency (k_{cat}/K_{M}) with NADH over k_{cat} with NADPH. In an exemplary embodiment, the modified or mutated KARI has at least about a 10:1 ratio of catalytic efficiency (k_{cat}/K_{M}) with NADH over catalytic efficiency (k_{cat}/K_{M}) with NADPH.

In one embodiment, the KARI exhibits at least about a 1:10 ratio of K_{M} for NADH over K_{M} for NADPH.

In additional embodiments, the application is directed to modified or mutated KARI enzymes that have been codon optimized for expression in certain desirable host organisms, such as yeast and E. coli.

In another aspect, the application relates to a recombinant microorganism comprising at least one nucleic acid molecule encoding a KARI enzyme, wherein said KARI enzyme has one or more modifications or mutations at positions corresponding to amino acids selected from: (a) tyrosine 35 of the S. exigua KARI (SEQ ID NO: 2); (b) leucine 57 of the S. exigua KARI (SEQ ID NO: 2); (c) arginine 58 of the S. exigua KARI (SEQ ID NO: 2); (d) glycine 60 of the S. exigua KARI (SEQ ID NO: 2); (e) serine 61 of the S. exigua KARI (SEQ ID NO: 2); (f) serine 62 of the S. exigua KARI (SEQ ID NO: 2); (g) serine 63 of the S. exigua KARI (SEQ ID NO: 2); (h) isoleucine 95 of the S. exigua KARI (SEQ ID NO: 2); and (i) valine 99 of the S. exigua KARI (SEQ ID NO: 2). Further included within the scope of the application are recombinant microorganisms comprising a KARI enzyme, other than the S. exigua KARI (SEQ ID NO: 2), which contains modifications or mutations at positions corresponding to those set out above.

In various embodiments described in the application, the recombinant microorganism comprises an isobutanol producing metabolic pathway. In one embodiment, the isobutanol producing metabolic pathway comprises at least one exogenous gene encoding a polypeptide that catalyzes a step in the conversion of pyruvate to isobutanol. In another embodiment, the isobutanol producing metabolic pathway comprises at least two exogenous genes encoding polypeptides that catalyze steps in the conversion of pyruvate to isobutanol. In yet another embodiment, the isobutanol producing metabolic pathway comprises at least three exogenous genes encoding polypeptides that catalyze steps in the conversion of pyruvate to isobutanol. In yet another embodiment, the isobutanol producing metabolic pathway comprises at least four exogenous genes encoding polypeptides that catalyze steps in the conversion of pyruvate to isobutanol. In yet another embodiment, the isobutanol producing metabolic pathway comprises at least five exogenous genes encoding polypeptides that catalyze steps in the conversion of pyruvate to isobutanol. In yet another embodiment, all of the isobutanol producing metabolic pathway steps in the conversion of pyruvate to isobutanol are converted by exogenously encoded enzymes. In an exemplary embodiment, at least one of the exogenously encoded enzymes is a KARI that is at least about 60% identical to SEQ ID NO: 2 and/or SEQ ID NO: 12. In another exemplary embodiment, at least one of the exogenously encoded enzymes is a KARI enzyme has one or more modifications or mutations at positions corresponding to amino acids selected from: (a) tyrosine 35 of the S. exigua KARI (SEQ ID NO: 2); (b) leucine 57 of the S. exigua KARI (SEQ ID NO: 2); (c) arginine 58 of the S. exigua KARI (SEQ ID NO: 2); (d) glycine 60 of the S. exigua KARI (SEQ ID NO: 2); (e) serine 61 of the S. exigua KARI (SEQ ID NO: 2); (f) serine 62 of the S. exigua KARI (SEQ ID NO: 2); (g) serine 63 of the S. exigua KARI (SEQ ID NO: 2): (h) isoleucine 95 of the S. exigua KARI (SEQ ID NO: 2); and (i) valine 99 of the S. exigua KARI (SEQ ID NO: 2).

In one embodiment, one or more of the isobutanol pathway genes encodes an enzyme that is localized to the cytosol. In one embodiment, the recombinant microorganisms comprise an isobutanol producing metabolic pathway with at least one isobutanol pathway enzyme localized in the cytosol. In another embodiment, the recombinant microorganisms comprise an isobutanol producing metabolic pathway with at least two isobutanol pathway enzymes localized in the cytosol. In yet another embodiment, the recombinant microorganisms comprise an isobutanol producing metabolic pathway with at least three isobutanol pathway enzymes localized in the cytosol. In yet another embodiment, the recombinant microorganisms comprise an isobutanol producing metabolic pathway with at least four isobutanol pathway enzymes localized in the cytosol. In an exemplary embodiment, the recombinant microorganisms comprise an isobutanol producing metabolic pathway with five isobutanol pathway enzymes localized in the cytosol. In yet another exemplary embodiment, the recombinant microorganisms comprise an isobutanol producing metabolic pathway with all isobutanol pathway enzymes localized in the cytosol.

In various embodiments described herein, the isobutanol pathway genes may encode enzyme(s) selected from the group consisting of acetolactate synthase (ALS), ketol-acid reductoisomerase (KARI), dihydroxyacid dehydratase (DHAD), 2-keto-acid decarboxylase, e.g., keto-isovalerate decarboxylase (KIVD), and alcohol dehydrogenase (ADH). In one embodiment, the KARI is an NADH-dependent KARI (NKR). In another embodiment, the ADH is an NADH-dependent ADH. In yet another embodiment, the KARI is an NADH-dependent KARI (NKR) and the ADH is an NADH-dependent ADH. In an exemplary embodiment, the KARI is at least about 60% identical to SEQ ID NO: 2 and/or SEQ ID NO: 12. In another exemplary embodiment, the KARI comprises one or more modifications or mutations at positions corresponding to amino acids selected from: (a) tyrosine 35 of the S. exigua KARI (SEQ ID NO: 2); (b) leucine 57 of the S. exigua KARI (SEQ ID NO: 2); (c) arginine 58 of the S. exigua KARI (SEQ ID NO: 2); (d) glycine 60 of the S. exigua KARI (SEQ ID NO: 2); (e) serine 61 of the S. exigua KARI (SEQ ID NO: 2); (f) serine 62 of the S. exigua KARI (SEQ ID NO: 2); (g) serine 63 of the S. exigua KARI (SEQ ID NO: 2); (h) isoleucine 95 of the S. exigua KARI (SEQ ID NO: 2); and (i) valine 99 of the S. exigua KARI (SEQ ID NO: 2).

In various embodiments described herein, the recombinant microorganisms of the invention that comprise an isobutanol producing metabolic pathway may be further engineered to reduce or eliminate the expression or activity of one or more enzymes selected from a pyruvate decarboxylase (PDC), a glycerol-3-phosphate dehydrogenase (GPD), a 3-keto acid reductase (3-KAR), or an aldehyde dehydrogenase (ALDH).

In various embodiments described herein, the recombinant microorganisms may be recombinant yeast microorganisms. In some embodiments, the recombinant yeast microorganisms may be members of the Saccharomyces clade, Saccharomyces sensu stricto microorganisms, Crabtree-negative yeast microorganisms, Crabtree-positive yeast microorganisms, post-WGD (whole genome duplication) yeast microorganisms, pre-WGD (whole genome duplication) yeast microorganisms, and non-fermenting yeast microorganisms.

In some embodiments, the recombinant microorganisms may be yeast recombinant microorganisms of the Saccharomyces clade.

In some embodiments, the recombinant microorganisms may be Saccharomyces sensu stricto microorganisms. In one embodiment, the Saccharomyces sensu stricto is selected from the group consisting of S. cerevisiae, S. kudriavzevii, S. mikatae, S. bayanus, S. uvarum, S. carocanis and hybrids thereof.

In some embodiments, the recombinant microorganisms may be Crabtree-negative recombinant yeast microorganisms. In one embodiment, the Crabtree-negative yeast microorganism is classified into a genera selected from the group consisting of Saccharomyces, Kluyveromyces, Pichia, Issatchenkia, Hansenula, or Candida. In additional embodiments, the Crabtree-negative yeast microorganism is selected from Saccharomyces kluyveri, Kluyveromyces lactis, Kluyveromyces marxianus, Pichia anomala, Pichia stipitis, Hansenula anomala, Candida utilis and Kluyveromyces waltii.

In some embodiments, the recombinant microorganisms may be Crabtree-positive recombinant yeast microorganisms. In one embodiment, the Crabtree-positive yeast microorganism is classified into a genera selected from the group consisting of Saccharomyces, Kluyveromyces, Zygosaccharomyces, Debaryomyces, Candida, Pichia and Schizosaccharomyces. In additional embodiments, the Crabtree-positive yeast microorganism is selected from the group consisting of Saccharomyces cerevisiae, Saccharomyces uvarum, Saccharomyces bayanus, Saccharomyces paradoxus, Saccharomyces castelli, Kluyveromyces thermotolerans, Candida glabrata, Z. bailli, Z. rouxii, Debaryomyces hansenii, Pichia pastorius, Schizosaccharomyces pombe, and Saccharomyces uvarum.

In some embodiments, the recombinant microorganisms may be post-WGD (whole genome duplication) yeast recombinant microorganisms. In one embodiment, the post-WGD yeast recombinant microorganism is classified into a genera selected from the group consisting of Saccharomyces or Candida. In additional embodiments, the post-WGD yeast is selected from the group consisting of Saccharomyces cerevisiae, Saccharomyces uvarum, Saccharomyces bayanus, Saccharomyces paradoxus, Saccharomyces castelli, and Candida glabrata.

In some embodiments, the recombinant microorganisms may be pre-WGD (whole genome duplication) yeast recombinant microorganisms. In one embodiment, the pre-WGD yeast recombinant microorganism is classified into a genera selected from the group consisting of Saccharomyces, Kluyveromyces, Candida, Pichia, Issatchenkia, Debaryomyces, Hansenula, Pachysolen, Yarrowia and Schizosaccharomyces. In additional embodiments, the pre-WGD yeast is selected from the group consisting of Saccharomyces kluyveri, Kluyveromyces thermotolerans, Kluyveromyces marxianus, Kluyveromyces waltii, Kluyveromyces lactis, Candida tropicalis, Pichia pastoris, Pichia anomala, Pichia stipitis, Issatchenkia orientalis, Issatchenkia occidentalis, Debaryomyces hansenii, Hansenula anomala, Pachysolen tannophilis, Yarrowia lipolytica, and Schizosaccharomyces pombe.

In some embodiments, the recombinant microorganisms may be microorganisms that are non-fermenting yeast microorganisms, including, but not limited to those, classified into a genera selected from the group consisting of Tricosporon, Rhodotorula, Myxozyma, or Candida. In a specific embodiment, the non-fermenting yeast is C. xestobii.

In another aspect, the present invention provides methods of producing isobutanol using a recombinant microorganism as described herein. In one embodiment, the method includes cultivating the recombinant microorganism in a culture medium containing a feedstock providing the carbon source until a recoverable quantity of isobutanol is produced and optionally, recovering the isobutanol. In one embodiment, the microorganism produces isobutanol from a carbon source at a yield of at least about 5 percent theoretical. In another embodiment, the microorganism produces isobutanol at a yield of at least about 10 percent, at least about 15 percent, about least about 20 percent, at least about 25 percent, at least about 30 percent, at least about 35 percent, at least about 40 percent, at least about 45 percent, at least about 50 percent, at least about 55 percent, at least about 60 percent, at least about 65 percent, at least about 70 percent, at least about 75 percent, at least about 80 percent, at least about 85 percent, at least about 90 percent, at least about 95 percent, or at least about 97.5 percent theoretical.

In one embodiment, the recombinant microorganism converts the carbon source to isobutanol under aerobic conditions. In another embodiment, the recombinant microorganism converts the carbon source to isobutanol under microaerobic conditions. In yet another embodiment, the recombinant microorganism converts the carbon source to isobutanol under anaerobic conditions.

### BRIEF DESCRIPTION OF DRAWINGS

Illustrative embodiments of the invention are illustrated in the drawings, in which:

FIG. 1 illustrates an exemplary embodiment of an isobutanol pathway.

FIG. 2 illustrates an exemplary embodiment of an NADH-dependent isobutanol pathway.

FIG. 3 illustrates the inhibition of NKR by 2,3-dihydroxyisovalerate (DHIV) using a dose response curve for the in vitro activity of an NADH-dependent version of the E. coli KARI in the presence of racemic DHIV.

FIG. 4 illustrates the optimum pH of the S. exigua KARI.

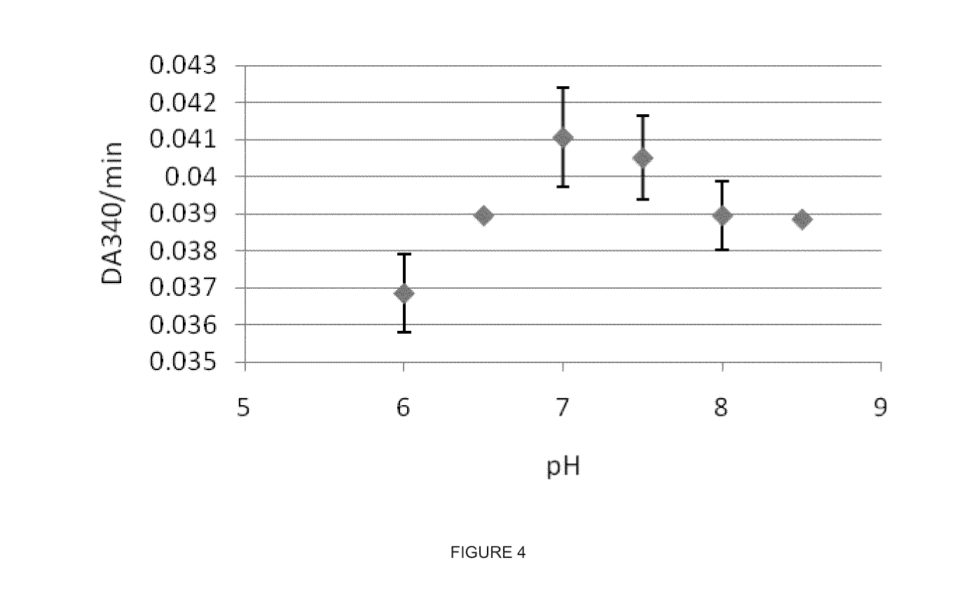

### DETAILED DESCRIPTION

As used herein and in the appended claims, the singular forms “a,” “an,” and “the” include plural referents unless the context clearly dictates otherwise. Thus, for example, reference to “a polynucleotide” includes a plurality of such polynucleotides and reference to “the microorganism” includes reference to one or more microorganisms, and so forth.

Unless defined otherwise, all technical and scientific terms used herein have the same meaning as commonly understood to one of ordinary skill in the art to which this disclosure belongs. Although methods and materials similar or equivalent to those described herein can be used in the practice of the disclosed methods and compositions, the exemplary methods, devices and materials are described herein.

Any publications discussed above and throughout the text are provided solely for their disclosure prior to the filing date of the present application. Nothing herein is to be construed as an admission that the inventors are not entitled to antedate such disclosure by virtue of prior disclosure.

The term “microorganism” includes prokaryotic and eukaryotic microbial species from the Domains Archaea, Bacteria and Eucarya, the latter including yeast and filamentous fungi, protozoa, algae, or higher Protista. The terms “microbial cells” and “microbes” are used interchangeably with the term microorganism.

The term “prokaryotes” is art recognized and refers to cells which contain no nucleus or other cell organelles. The prokaryotes are generally classified in one of two domains, the Bacteria and the Archaea. The definitive difference between organisms of the Archaea and Bacteria domains is based on fundamental differences in the nucleotide base sequence in the 16S ribosomal RNA.

The term “Archaea” refers to a categorization of organisms of the division Mendosicutes, typically found in unusual environments and distinguished from the rest of the prokaryotes by several criteria, including the number of ribosomal proteins and the lack of muramic acid in cell walls. On the basis of ssrRNA analysis, the Archaea consist of two phylogenetically-distinct groups: Crenarchaeota and Euryarchaeota. On the basis of their physiology, the Archaea can be organized into three types: methanogens (prokaryotes that produce methane); extreme halophiles (prokaryotes that live at very high concentrations of salt (NaCl); and extreme (hyper) thermophiles (prokaryotes that live at very high temperatures). Besides the unifying archaeal features that distinguish them from Bacteria (i.e., no murein in cell wall, ester-linked membrane lipids, etc.), these prokaryotes exhibit unique structural or biochemical attributes which adapt them to their particular habitats. The Crenarchaeota consist mainly of hyperthermophilic sulfur-dependent prokaryotes and the Euryarchaeota contain the methanogens and extreme halophiles.

“Bacteria”, or “eubacteria”, refers to a domain of prokaryotic organisms. Bacteria include at least eleven distinct groups as follows: (1) Gram-positive (gram+) bacteria, of which there are two major subdivisions: (1) high G+C group (Actinomycetes, Mycobacteria, Micrococcus, others) (2) low G+C group (Bacillus, Clostridia, Lactobacillus, Staphylococci, Streptococci, Mycoplasmas); (2) Proteobacteria, e.g., Purple photosynthetic+non-photosynthetic Gram-negative bacteria (includes most “common” Gram-negative bacteria); (3) Cyanobacteria, e.g., oxygenic phototrophs; (4) Spirochetes and related species; (5) Planctomyces; (6) Bacteroides, Flavobacteria; (7) Chlamydia; (8) Green sulfur bacteria; (9) Green non-sulfur bacteria (also anaerobic phototrophs); (10) Radioresistant micrococci and relatives; (11) Thermotoga and Thermosipho thermophiles.

“Gram-negative bacteria” include cocci, nonenteric rods, and enteric rods. The genera of Gram-negative bacteria include, for example, Neisseria, Spirillum, Pasteurella, Brucella, Yersinia, Francisella, Haemophilus, Bordetella, Escherichia, Salmonella, Shigella, Klebsiella, Proteus, Vibrio, Pseudomonas, Bacteroides, Acetobacter, Aerobacter, Agrobacterium, Azotobacter, Spirilla, Serratia, Vibrio, Rhizobium, Chlamydia, Rickettsia, Treponema, and Fusobacterium.

“Gram positive bacteria” include cocci, nonsporulating rods, and sporulating rods. The genera of gram positive bacteria include, for example, Actinomyces, Bacillus, Clostridium, Corynebacterium, Erysipelothrix, Lactobacillus, Listeria, Mycobacterium, Myxococcus, Nocardia, Staphylococcus, Streptococcus, and Streptomyces.

The term “genus” is defined as a taxonomic group of related species according to the Taxonomic Outline of Bacteria and Archaea (Garrity, G. M., Lilbum, T. G., Cole, J. R., Harrison, S. H., Euzeby, J., and Tindall, B. J. (2007) The Taxonomic Outline of Bacteria and Archaea. TOBA Release 7.7, March 2007. Michigan State University Board of Trustees.

The term “species” is defined as a collection of closely related organisms with greater than 97% 16S ribosomal RNA sequence homology and greater than 70% genomic hybridization and sufficiently different from all other organisms so as to be recognized as a distinct unit.

The terms “recombinant microorganism,” “modified microorganism,” and “recombinant host cell” are used interchangeably herein and refer to microorganisms that have been genetically modified to express or to overexpress endogenous polynucleotides, to express heterologous polynucleotides, such as those included in a vector, in an integration construct, or which have an alteration in expression of an endogenous gene. By “alteration” it is meant that the expression of the gene, or level of a RNA molecule or equivalent RNA molecules encoding one or more polypeptides or polypeptide subunits, or activity of one or more polypeptides or polypeptide subunits is up regulated or down regulated, such that expression, level, or activity is greater than or less than that observed in the absence of the alteration. For example, the term “alter” can mean “inhibit,” but the use of the word “alter” is not limited to this definition. It is understood that the terms “recombinant microorganism” and “recombinant host cell” refer not only to the particular recombinant microorganism but to the progeny or potential progeny of such a microorganism. Because certain modifications may occur in succeeding generations due to either mutation or environmental influences, such progeny may not, in fact, be identical to the parent cell, but are still included within the scope of the term as used herein.

The term “expression” with respect to a gene sequence refers to transcription of the gene and, as appropriate, translation of the resulting mRNA transcript to a protein. Thus, as will be clear from the context, expression of a protein results from transcription and translation of the open reading frame sequence. The level of expression of a desired product in a host cell may be determined on the basis of either the amount of corresponding mRNA that is present in the cell, or the amount of the desired product encoded by the selected sequence. For example, mRNA transcribed from a selected sequence can be quantitated by qRT-PCR or by Northern hybridization (see Sambrook et al., Molecular Cloning: A Laboratory Manual, Cold Spring Harbor Laboratory Press (1989)). Protein encoded by a selected sequence can be quantitated by various methods, e.g., by ELISA, by assaying for the biological activity of the protein, or by employing assays that are independent of such activity, such as western blotting or radioimmunoassay, using antibodies that recognize and bind the protein. See Sambrook et al., 1989, supra.

The term “overexpression” refers to an elevated level (e.g., aberrant level) of mRNAs encoding for a protein(s), and/or to elevated levels of protein(s) in cells as compared to similar corresponding unmodified cells expressing basal levels of mRNAs or having basal levels of proteins. In particular embodiments, mRNA(s) or protein(s) may be overexpressed by at least 2-fold, 3-fold, 4-fold, 5-fold, 6-fold, 8-fold, 10-fold, 12-fold, 15-fold or more in microorganisms engineered to exhibit increased gene mRNA, protein, and/or activity.

As used herein and as would be understood by one of ordinary skill in the art, “reduced activity and/or expression” of a protein such as an enzyme can mean either a reduced specific catalytic activity of the protein (e.g. reduced activity) and/or decreased concentrations of the protein in the cell (e.g. reduced expression). As would be understood by one or ordinary skill in the art, the reduced activity of a protein in a cell may result from decreased concentrations of the protein in the cell.

The term “wild-type microorganism” describes a cell that occurs in nature, i.e. a cell that has not been genetically modified. A wild-type microorganism can be genetically modified to express or overexpress a first target enzyme. This microorganism can act as a parental microorganism in the generation of a microorganism modified to express or overexpress a second target enzyme. In turn, the microorganism modified to express or overexpress a first and a second target enzyme can be modified to express or overexpress a third target enzyme.

Accordingly, a “parental microorganism” functions as a reference cell for successive genetic modification events. Each modification event can be accomplished by introducing a nucleic acid molecule in to the reference cell. The introduction facilitates the expression or overexpression of a target enzyme. It is understood that the term “facilitates” encompasses the activation of endogenous polynucleotides encoding a target enzyme through genetic modification of e.g., a promoter sequence in a parental microorganism. It is further understood that the term “facilitates” encompasses the introduction of heterologous polynucleotides encoding a target enzyme in to a parental microorganism

The term “engineer” refers to any manipulation of a microorganism that results in a detectable change in the microorganism, wherein the manipulation includes but is not limited to inserting a polynucleotide and/or polypeptide heterologous to the microorganism and mutating a polynucleotide and/or polypeptide native to the microorganism.

The term “mutation” as used herein indicates any modification of a nucleic acid and/or polypeptide which results in an altered nucleic acid or polypeptide. Mutations include, for example, point mutations, deletions, or insertions of single or multiple residues in a polynucleotide, which includes alterations arising within a protein-encoding region of a gene as well as alterations in regions outside of a protein-encoding sequence, such as, but not limited to, regulatory or promoter sequences. A genetic alteration may be a mutation of any type. For instance, the mutation may constitute a point mutation, a frame-shift mutation, a nonsense mutation, an insertion, or a deletion of part or all of a gene. In addition, in some embodiments of the modified microorganism, a portion of the microorganism genome has been replaced with a heterologous polynucleotide. In some embodiments, the mutations are naturally-occurring. In other embodiments, the mutations are identified and/or enriched through artificial selection pressure. In still other embodiments, the mutations in the microorganism genome are the result of genetic engineering.

The term “biosynthetic pathway”, also referred to as “metabolic pathway”, refers to a set of anabolic or catabolic biochemical reactions for converting one chemical species into another. Gene products belong to the same “metabolic pathway” if they, in parallel or in series, act on the same substrate, produce the same product, or act on or produce a metabolic intermediate (i.e., metabolite) between the same substrate and metabolite end product.

As used herein, the term “isobutanol producing metabolic pathway” refers to an enzyme pathway which produces isobutanol from pyruvate.

The term “NADH-dependent” as used herein with reference to an enzyme, e.g., KARI and/or ADH, refers to an enzyme that catalyzes the reduction of a substrate coupled to the oxidation of NADH with a catalytic efficiency that is greater than the reduction of the same substrate coupled to the oxidation of NADPH at equal substrate and cofactor concentrations.

The term “exogenous” as used herein with reference to various molecules, e.g., polynucleotides, polypeptides, enzymes, etc., refers to molecules that are not normally or naturally found in and/or produced by a given yeast, bacterium, organism, microorganism, or cell in nature.

On the other hand, the term “endogenous” or “native” as used herein with reference to various molecules, e.g., polynucleotides, polypeptides, enzymes, etc., refers to molecules that are normally or naturally found in and/or produced by a given yeast, bacterium, organism, microorganism, or cell in nature.

The term “heterologous” as used herein in the context of a modified host cell refers to various molecules, e.g., polynucleotides, polypeptides, enzymes, etc., wherein at least one of the following is true: (a) the molecule(s) is/are foreign (“exogenous”) to (i.e., not naturally found in) the host cell; (b) the molecule(s) is/are naturally found in (e.g., is “endogenous to”) a given host microorganism or host cell but is either produced in an unnatural location or in an unnatural amount in the cell; and/or (c) the molecule(s) differ(s) in nucleotide or amino acid sequence from the endogenous nucleotide or amino acid sequence(s) such that the molecule differing in nucleotide or amino acid sequence from the endogenous nucleotide or amino acid as found endogenously is produced in an unnatural (e.g., greater than naturally found) amount in the cell.

The term “feedstock” is defined as a raw material or mixture of raw materials supplied to a microorganism or fermentation process from which other products can be made. For example, a carbon source, such as biomass or the carbon compounds derived from biomass are a feedstock for a microorganism that produces a biofuel in a fermentation process. However, a feedstock may contain nutrients other than a carbon source.

The term “substrate” or “suitable substrate” refers to any substance or compound that is converted or meant to be converted into another compound by the action of an enzyme. The term includes not only a single compound, but also combinations of compounds, such as solutions, mixtures and other materials which contain at least one substrate, or derivatives thereof. Further, the term “substrate” encompasses not only compounds that provide a carbon source suitable for use as a starting material, such as any biomass derived sugar, but also intermediate and end product metabolites used in a pathway associated with a recombinant microorganism as described herein.

The term “fermentation” or “fermentation process” is defined as a process in which a microorganism is cultivated in a culture medium containing raw materials, such as feedstock and nutrients, wherein the microorganism converts raw materials, such as a feedstock, into products.

The term “volumetric productivity” or “production rate” is defined as the amount of product formed per volume of medium per unit of time. Volumetric productivity is reported in gram per liter per hour (g/L/h).

The term “specific productivity” or “specific production rate” is defined as the amount of product formed per volume of medium per unit of time per amount of cells. Specific productivity is reported in gram (or milligram) per gram cell dry weight per hour (g/g h).

The term “yield” is defined as the amount of product obtained per unit weight of raw material and may be expressed as g product per g substrate (g/g). Yield may be expressed as a percentage of the theoretical yield. “Theoretical yield” is defined as the maximum amount of product that can be generated per a given amount of substrate as dictated by the stoichiometry of the metabolic pathway used to make the product. For example, the theoretical yield for one typical conversion of glucose to isobutanol is 0.41 g/g. As such, a yield of isobutanol from glucose of 0.39 g/g would be expressed as 95% of theoretical or 95% theoretical yield.

The term “titer” is defined as the strength of a solution or the concentration of a substance in solution. For example, the titer of a biofuel in a fermentation broth is described as g of biofuel in solution per liter of fermentation broth (g/L).

“Aerobic conditions” are defined as conditions under which the oxygen concentration in the fermentation medium is sufficiently high for an aerobic or facultative anaerobic microorganism to use as a terminal electron acceptor.

In contrast, “anaerobic conditions” are defined as conditions under which the oxygen concentration in the fermentation medium is too low for the microorganism to use as a terminal electron acceptor. Anaerobic conditions may be achieved by sparging a fermentation medium with an inert gas such as nitrogen until oxygen is no longer available to the microorganism as a terminal electron acceptor. Alternatively, anaerobic conditions may be achieved by the microorganism consuming the available oxygen of the fermentation until oxygen is unavailable to the microorganism as a terminal electron acceptor. Methods for the production of isobutanol under anaerobic conditions are described in commonly owned and co-pending publication, US 2010/0143997, the disclosures of which are herein incorporated by reference in its entirety for all purposes.

“Aerobic metabolism” refers to a biochemical process in which oxygen is used as a terminal electron acceptor to make energy, typically in the form of ATP, from carbohydrates. Aerobic metabolism occurs, e.g., via glycolysis and the TCA cycle, wherein a single glucose molecule is metabolized completely into carbon dioxide in the presence of oxygen.

In contrast, “anaerobic metabolism” refers to a biochemical process in which oxygen is not the final acceptor of electrons contained in NADH. Anaerobic metabolism can be divided into anaerobic respiration, in which compounds other than oxygen serve as the terminal electron acceptor, and substrate level phosphorylation, in which the electrons from NADH are utilized to generate a reduced product via a “fermentative pathway.”

In “fermentative pathways”, NAD(P)H donates its electrons to a molecule produced by the same metabolic pathway that produced the electrons carried in NAD(P)H. For example, in one of the fermentative pathways of certain yeast strains, NAD(P)H generated through glycolysis transfers its electrons to pyruvate, yielding ethanol. Fermentative pathways are usually active under anaerobic conditions but may also occur under aerobic conditions, under conditions where NADH is not fully oxidized via the respiratory chain. For example, above certain glucose concentrations, Crabtree positive yeasts produce large amounts of ethanol under aerobic conditions.

The term “byproduct” or “by-product” means an undesired product related to the production of an amino acid, amino acid precursor, chemical, chemical precursor, biofuel, biofuel precursor, higher alcohol, or higher alcohol precursor.

The term “substantially free” when used in reference to the presence or absence of a protein activity (3-KAR enzymatic activity, ALDH enzymatic activity, PDC enzymatic activity, GPD enzymatic activity, etc.) means the level of the protein is substantially less than that of the same protein in the wild-type host, wherein less than about 50% of the wild-type level is preferred and less than about 30% is more preferred. The activity may be less than about 20%, less than about 10%, less than about 5%, or less than about 1% of wild-type activity. Microorganisms which are “substantially free” of a particular protein activity (3-KAR enzymatic activity, ALDH enzymatic activity, PDC enzymatic activity, GPD enzymatic activity, etc.) may be created through recombinant means or identified in nature.

The term “non-fermenting yeast” is a yeast species that fails to demonstrate an anaerobic metabolism in which the electrons from NADH are utilized to generate a reduced product via a fermentative pathway such as the production of ethanol and CO₂ from glucose. Non-fermentative yeast can be identified by the “Durham Tube Test” (J. A. Barnett, R. W. Payne, and D. Yarrow. 2000. Yeasts Characteristics and Identification. 3^{rd} edition. p. 28-29. Cambridge University Press, Cambridge, UK) or by monitoring the production of fermentation productions such as ethanol and CO₂.

The term “polynucleotide” is used herein interchangeably with the term “nucleic acid” and refers to an organic polymer composed of two or more monomers including nucleotides, nucleosides or analogs thereof, including but not limited to single stranded or double stranded, sense or antisense deoxyribonucleic acid (DNA) of any length and, where appropriate, single stranded or double stranded, sense or antisense ribonucleic acid (RNA) of any length, including siRNA. The term “nucleotide” refers to any of several compounds that consist of a ribose or deoxyribose sugar joined to a purine or a pyrimidine base and to a phosphate group, and that are the basic structural units of nucleic acids. The term “nucleoside” refers to a compound (as guanosine or adenosine) that consists of a purine or pyrimidine base combined with deoxyribose or ribose and is found especially in nucleic acids. The term “nucleotide analog” or “nucleoside analog” refers, respectively, to a nucleotide or nucleoside in which one or more individual atoms have been replaced with a different atom or with a different functional group. Accordingly, the term polynucleotide includes nucleic acids of any length, DNA, RNA, analogs and fragments thereof. A polynucleotide of three or more nucleotides is also called nucleotidic oligomer or oligonucleotide.

It is understood that the polynucleotides described herein include “genes” and that the nucleic acid molecules described herein include “vectors” or “plasmids.” Accordingly, the term “gene”, also called a “structural gene” refers to a polynucleotide that codes for a particular sequence of amino acids, which comprise all or part of one or more proteins or enzymes, and may include regulatory (non-transcribed) DNA sequences, such as promoter sequences, which determine for example the conditions under which the gene is expressed. The transcribed region of the gene may include untranslated regions, including introns, 5′-untranslated region (UTR), and 3′-UTR, as well as the coding sequence.

The term “operon” refers to two or more genes which are transcribed as a single transcriptional unit from a common promoter. In some embodiments, the genes comprising the operon are contiguous genes. It is understood that transcription of an entire operon can be modified (i.e., increased, decreased, or eliminated) by modifying the common promoter. Alternatively, any gene or combination of genes in an operon can be modified to alter the function or activity of the encoded polypeptide. The modification can result in an increase in the activity of the encoded polypeptide. Further, the modification can impart new activities on the encoded polypeptide. Exemplary new activities include the use of alternative substrates and/or the ability to function in alternative environmental conditions.

A “vector” is any means by which a nucleic acid can be propagated and/or transferred between organisms, cells, or cellular components. Vectors include viruses, bacteriophage, pro-viruses, plasmids, phagemids, transposons, and artificial chromosomes such as YACs (yeast artificial chromosomes), BACs (bacterial artificial chromosomes), and PLACs (plant artificial chromosomes), and the like, that are “episomes,” that is, that replicate autonomously or can integrate into a chromosome of a host cell. A vector can also be a naked RNA polynucleotide, a naked DNA polynucleotide, a polynucleotide composed of both DNA and RNA within the same strand, a poly-lysine-conjugated DNA or RNA, a peptide-conjugated DNA or RNA, a liposome-conjugated DNA, or the like, that are not episomal in nature, or it can be an organism which comprises one or more of the above polynucleotide constructs such as an agrobacterium or a bacterium.

“Transformation” refers to the process by which a vector is introduced into a host cell. Transformation (or transduction, or transfection), can be achieved by any one of a number of means including chemical transformation (e.g. lithium acetate transformation), electroporation, microinjection, biolistics (or particle bombardment-mediated delivery), or agrobacterium mediated transformation.

The term “enzyme” as used herein refers to any substance that catalyzes or promotes one or more chemical or biochemical reactions, which usually includes enzymes totally or partially composed of a polypeptide or polypeptides, but can include enzymes composed of a different molecule including polynucleotides.

The term “protein,” “peptide,” or “polypeptide” as used herein indicates an organic polymer composed of two or more amino acidic monomers and/or analogs thereof. As used herein, the term “amino acid” or “amino acidic monomer” refers to any natural and/or synthetic amino acids including glycine and both D or L optical isomers. The term “amino acid analog” refers to an amino acid in which one or more individual atoms have been replaced, either with a different atom, or with a different functional group. Accordingly, the term polypeptide includes amino acidic polymer of any length including full length proteins, and peptides as well as analogs and fragments thereof. A polypeptide of three or more amino acids is also called a protein oligomer or oligopeptide

The term “homolog,” used with respect to an original polynucleotide or polypeptide of a first family or species, refers to distinct polynucleotides or polypeptides of a second family or species which are determined by functional, structural or genomic analyses to be a polynucleotide or polypeptide of the second family or species which corresponds to the original polynucleotide or polypeptide of the first family or species. Most often, homologs will have functional, structural or genomic similarities. Techniques are known by which homologs of a polynucleotide or polypeptide can readily be cloned using genetic probes and PCR. Identity of cloned sequences as homolog can be confirmed using functional assays and/or by genomic mapping of the genes.

A polypeptide has “homology” or is “homologous” to a second polypeptide if the amino acid sequence encoded by a gene has a similar amino acid sequence to that of the second gene. Alternatively, a polypeptide has homology to a second polypeptide if the two polypeptides have “similar” amino acid sequences. (Thus, the terms “homologous polypeptides” or “homologous proteins” are defined to mean that the two polypeptides have similar amino acid sequences).

The term “analog” or “analogous” refers to polynucleotide or polypeptide sequences that are related to one another in function only and are not from common descent or do not share a common ancestral sequence. Analogs may differ in sequence but may share a similar structure, due to convergent evolution. For example, two enzymes are analogs or analogous if the enzymes catalyze the same reaction of conversion of a substrate to a product, are unrelated in sequence, and irrespective of whether the two enzymes are related in structure.

### Isobutanol Producing Recombinant Microorganisms

A variety of microorganisms convert sugars to produce pyruvate, which is then utilized in a number of pathways of cellular metabolism. In recent years, microorganisms, including yeast, have been engineered to produce a number of desirable products via pyruvate-driven biosynthetic pathways, including isobutanol, an important commodity chemical and biofuel candidate (See, e.g., commonly owned and co-pending patent publications, US 2009/0226991, US 2010/0143997, US 2011/0020889, US 2011/0076733, and WO 2010/075504).

As described herein, the present invention relates to recombinant microorganisms for producing isobutanol, wherein said recombinant microorganisms comprise an isobutanol producing metabolic pathway. In one embodiment, the isobutanol producing metabolic pathway to convert pyruvate to isobutanol can be comprised of the following reactions:

1. 2 pyruvate→acetolactate+CO₂

2. acetolactate+NAD(P)H→2,3-dihydroxyisovalerate+NAD(P)⁺

3. 2,3-dihydroxyisovalerate→alpha-ketoisovalerate

4. alpha-ketoisovalerate→isobutyraldehyde+CO₂

5. isobutyraldehyde+NAD(P)H→isobutanol+NADP

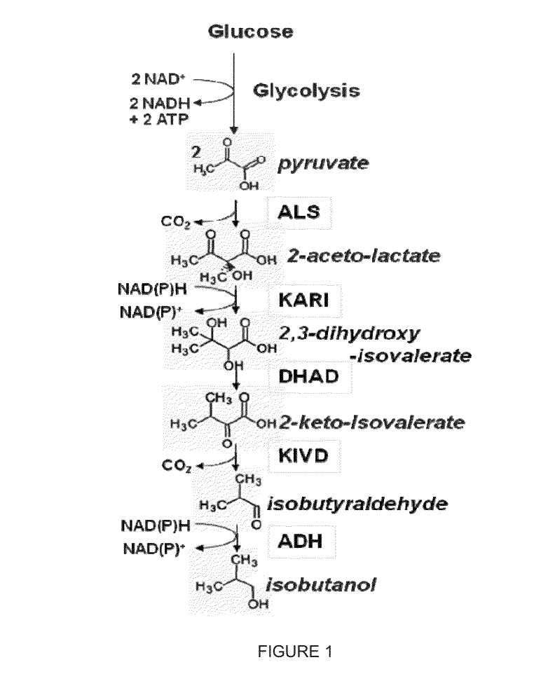

In one embodiment, these reactions are carried out by the enzymes 1) Acetolactate synthase (ALS), 2) Ketol-acid reductoisomerase (KARI), 3) Dihydroxyacid dehydratase (DHAD), 4) 2-keto-acid decarboxylase, e.g., Keto-isovalerate decarboxylase (KIVD), and 5) an Alcohol dehydrogenase (ADH) (FIG. 1). In some embodiments, the recombinant microorganism may be engineered to overexpress one or more of these enzymes. In an exemplary embodiment, the recombinant microorganism is engineered to overexpress all of these enzymes.

Alternative pathways for the production of isobutanol in yeast have been described in WO/2007/050671 and in Dickinson et al., 1998, J Biol Chem 273:25751-6. These and other isobutanol producing metabolic pathways are within the scope of the present application. In one embodiment, the isobutanol producing metabolic pathway comprises five substrate to product reactions. In another embodiment, the isobutanol producing metabolic pathway comprises six substrate to product reactions. In yet another embodiment, the isobutanol producing metabolic pathway comprises seven substrate to product reactions.

In various embodiments described herein, the recombinant microorganism comprises an isobutanol producing metabolic pathway. In one embodiment, the isobutanol producing metabolic pathway comprises at least one exogenous gene encoding a polypeptide that catalyzes a step in the conversion of pyruvate to isobutanol. In another embodiment, the isobutanol producing metabolic pathway comprises at least two exogenous genes encoding polypeptides that catalyze steps in the conversion of pyruvate to isobutanol. In yet another embodiment, the isobutanol producing metabolic pathway comprises at least three exogenous genes encoding polypeptides that catalyze steps in the conversion of pyruvate to isobutanol. In yet another embodiment, the isobutanol producing metabolic pathway comprises at least four exogenous genes encoding polypeptides that catalyze steps in the conversion of pyruvate to isobutanol. In yet another embodiment, the isobutanol producing metabolic pathway comprises at least five exogenous genes encoding polypeptides that catalyze steps in the conversion of pyruvate to isobutanol. In yet another embodiment, all of the isobutanol producing metabolic pathway steps in the conversion of pyruvate to isobutanol are converted by exogenously encoded enzymes.

In one embodiment, one or more of the isobutanol pathway genes encodes an enzyme that is localized to the cytosol. In one embodiment, the recombinant microorganisms comprise an isobutanol producing metabolic pathway with at least one isobutanol pathway enzyme localized in the cytosol. In another embodiment, the recombinant microorganisms comprise an isobutanol producing metabolic pathway with at least two isobutanol pathway enzymes localized in the cytosol. In yet another embodiment, the recombinant microorganisms comprise an isobutanol producing metabolic pathway with at least three isobutanol pathway enzymes localized in the cytosol. In yet another embodiment, the recombinant microorganisms comprise an isobutanol producing metabolic pathway with at least four isobutanol pathway enzymes localized in the cytosol. In an exemplary embodiment, the recombinant microorganisms comprise an isobutanol producing metabolic pathway with five isobutanol pathway enzymes localized in the cytosol. In yet another exemplary embodiment, the recombinant microorganisms comprise an isobutanol producing metabolic pathway with all isobutanol pathway enzymes localized in the cytosol. Isobutanol producing metabolic pathways in which one or more genes are localized to the cytosol are described in commonly owned and co-pending U.S. application Ser. No. 12/855,276, which is herein incorporated by reference in its entirety for all purposes.

As is understood in the art, a variety of organisms can serve as sources for the isobutanol pathway enzymes, including, but not limited to, Saccharomyces spp., including S. cerevisiae and S. uvarum, Kluyveromyces spp., including K. thermotolerans. K. lactis, and K. marxianus, Pichia spp., Hansenula spp., including H. polymorpha, Candida spp., Trichosporon spp., Yamadazyma spp., including Y. spp. stipitis, Torulaspora pretoriensis, Issatchenkia orientalis, Schizosaccharomyces spp., including S. pombe, Cryptococcus spp., Aspergillus spp., Neurospora spp., or Ustilago spp. Sources of genes from anaerobic fungi include, but not limited to, Piromyces spp., Orpinomyces spp., or Neocallimastix spp. Sources of prokaryotic enzymes that are useful include, but not limited to, Escherichia spp., Zymomonas spp., Staphylococcus spp., Bacillus spp., Clostridium spp., Corynebacterium spp., Pseudomonas spp., Lactococcus spp., Enterobacter spp., Streptococcus spp., Salmonella spp., Slackia spp., Cryptobacterium spp., and Eggerthella spp.

In some embodiments, one or more of these enzymes can be encoded by native genes. Alternatively, one or more of these enzymes can be encoded by heterologous genes.

For example, acetolactate synthases capable of converting pyruvate to acetolactate may be derived from a variety of sources (e.g., bacterial, yeast, Archaea, etc.), including B. subtilis (GenBank Accession No. Q04789.3). L. lactis (GenBank Accession No. NP_{—}267340.1), S. mutans (GenBank Accession No. NP_{—}721805.1), K. pneumoniae (GenBank Accession No. ZP_{—}06014957.1), C. glutamicum (GenBank Accession No. P42463.1), E. cloacae (GenBank Accession No. YP_{—}003613611.1), M. maripaludis (GenBank Accession No. ABX01060.1), M. grisea (GenBank Accession No. AAB81248.1), T. stipitatus (GenBank Accession No. XP_{—}002485976.1), or S. cerevisiae ILV2 (GenBank Accession No. NP_{—}013826.1). Additional acetolactate synthases capable of converting pyruvate to acetolactate are described in commonly owned and co-pending US Publication No. 2011/0076733, which is herein incorporated by reference in its entirety. A review article characterizing the biosynthesis of acetolactate from pyruvate via the activity of acetolactate synthases is provided by Chipman et al., 1998, Biochimica et Biophysica Acta 1385: 401-19, which is herein incorporated by reference in its entirety. Chipman et al. provide an alignment and consensus for the sequences of a representative number of acetolactate synthases. Motifs shared in common between the majority of acetolactate synthases include:

(SEQ ID NO: 13)
SGPG(A/C/V)(T/S)N,
(SEQ ID NO: 14)
GX(P/A)GX(V/A/T),
(SEQ ID NO: 15)
GX(Q/G)(T/A)(L/M)G(Y/F/W)(A/G)
X(P/G)(W/A)AX(G/T)(A/V),
and
(SEQ ID NO: 16)
GD(G/A)(G/S/C)F

motifs at amino acid positions corresponding to the 163-169, 240-245, 521-535, and 549-553 residues, respectively, of the S. cerevisiae ILV2. Thus, a protein harboring one or more of these amino acid motifs can generally be expected to exhibit acetolactate synthase activity.

Dihydroxy acid dehydratases capable of converting 2,3-dihydroxyisovalerate to α-ketoisovalerate may be derived from a variety of sources (e.g., bacterial, yeast, Archaea, etc.), including E. coli (GenBank Accession No. YP_{—}026248.1), L. lactis (GenBank Accession No. NP_{—}267379.1), S. mutans (GenBank Accession No. NP_{—}722414.1), M. stadtmanae (GenBank Accession No. YP_{—}448586.1), M. tractuosa (GenBank Accession No. YP_{—}004053736.1), Eubacterium SCB49 (GenBank Accession No. ZP_{—}01890126.1), G. forsetti (GenBank Accession No. YP_{—}862145.1), Y. lipolytica (GenBank Accession No. XP_{—}502180.2), N. crassa (GenBank Accession No. XP_{—}963045.1), or S. cerevisiae ILV3 (GenBank Accession No. NP_{—}012550.1). Additional dihydroxy acid dehydratases capable of 2,3-dihydroxyisovalerate to α-ketoisovalerate are described in commonly owned and co-pending US Publication No. 2011/0076733. Motifs shared in common between the majority of dihydroxy acid dehydratases include:

(SEQ ID NO: 17)
SLXSRXXIA,
(SEQ ID NO: 18)
CDKXXPG,
(SEQ ID NO: 19)
GXCXGXXTAN,
(SEQ ID NO: 20)
GGSTN,
(SEQ ID NO: 21)
GPXGXPGMRXE,
(SEQ ID NO: 22)
ALXTDGRXSG,
and
(SEQ ID NO: 23)
GHXXPEA

motifs at amino acid positions corresponding to the 93-101, 122-128, 193-202, 276-280, 482-491, 509-518, and 526-532 residues, respectively, of the E. coli dihydroxy acid dehydratase encoded by ilvD. Thus, a protein harboring one or more of these amino acid motifs can generally be expected to exhibit dihydroxy acid dehydratase activity.

2-keto-acid decarboxylases capable of converting α-ketoisovalerate to isobutyraldehyde may be derived from a variety of sources (e.g., bacterial, yeast, Archaea, etc.), including L. lactis kivD (GenBank Accession No. YP_{—}003353820.1), E. cloacae (GenBank Accession No. P23234.1). M. smegmatis (GenBank Accession No. A0R480.1), M. tuberculosis (GenBank Accession No. 053865.1), M. avium (GenBank Accession No. Q742Q2.1, A. brasilense (GenBank Accession No. P51852.1), L. lactis kdcA (GenBank Accession No. AAS49166.1), S. epidermidis (GenBank Accession No. NP_{—}765765.1), M. caseolyticus (GenBank Accession No. YP_{—}002560734.1), B. megaterium (GenBank Accession No. YP_{—}003561644.1), S. cerevisiae ARO10 (GenBank Accession No. NP_{—}010668.1), or S. cerevisiae THI3 (GenBank Accession No. CAA98646.1). Additional 2-keto-acid decarboxylases capable of converting α-ketoisovalerate to isobutyraldehyde are described in commonly owned and co-pending US Publication No. 2011/0076733. Motifs shared in common between the majority of 2-keto-acid decarboxylases include:

(SEQ ID NO: 24)
FG(V/I)(P/S)G(D/E)(Y/F),
(SEQ ID NO: 25)
(T/V)T(F/Y)G(V/A)G(E/A)(L/F)(S/N),
(SEQ ID NO: 26)
N(G/A)(L/I/V)AG(S/A)(Y/F)AE,
(SEQ ID NO: 27)
(V/I)(L/I/V)XI(V/T/S)G,
and
(SEQ ID NO: 28)
GDG(S/A)(L/F/A)Q(L/M)T

motifs at amino acid positions corresponding to the 21-27, 70-78, 81-89, 93-98, and 428-435 residues, respectively, of the L. lactis 2-keto-acid decarboxylase encoded by kivD. Thus, a protein harboring one or more of these amino acid motifs can generally be expected to exhibit 2-keto-acid decarboxylase activity.

Alcohol dehydrogenases capable of converting isobutyraldehyde to isobutanol may be derived from a variety of sources (e.g., bacterial, yeast, Archaea, etc.), including L. lactis (GenBank Accession No. YP_{—}003354381), B. cereus (GenBank Accession No. YP_{—}001374103.1), N. meningitidis (GenBank Accession No. CBA03965.1), S. sanguinis (GenBank Accession No. YP_{—}001035842.1), L. brevis (GenBank Accession No. YP_{—}794451.1), B. thuringiensis (GenBank Accession No. ZP_{—}04101989.1), P. acidilactici (GenBank Accession No. ZP_{—}06197454.1), B. subtilis (GenBank Accession No. EHA31115.1), N. crassa (GenBank Accession No. CAB91241.1) or S. cerevisiae ADH6 (GenBank Accession No. NP_{—}014051.1). Additional alcohol dehydrogenases capable of converting isobutyraldehyde to isobutanol are described in commonly owned and co-pending US Publication Nos. 2011/0076733 and 2011/0201072. Motifs shared in common between the majority of alcohol dehydrogenases include:

(SEQ ID NO: 29)
C(H/G)(T/S)D(L/I)H,
(SEQ ID NO: 30)
GHEXXGXV,
(SEQ ID NO: 31)
(L/V)(Q/K/E)(V/I/K)G(D/Q)(R/H)(V/A),
(SEQ ID NO: 32)
CXXCXXC,
(SEQ ID NO: 33)
(C/A)(A/G/D)(G/A)XT(T/V),
and
(SEQ ID NO: 34)
G(L/A/C)G(G/P)(L/I/V)G

motifs at amino acid positions corresponding to the 39-44, 59-66, 76-82, 91-97, 147-152, and 171-176 residues, respectively, of the L. lactis alcohol dehydrogenase encoded by adhA. Thus, a protein harboring one or more of these amino acid motifs can generally be expected to exhibit alcohol dehydrogenase activity.

In another embodiment, the yeast microorganism may be engineered to have increased ability to convert pyruvate to isobutanol. In one embodiment, the yeast microorganism may be engineered to have increased ability to convert pyruvate to isobutyraldehyde. In another embodiment, the yeast microorganism may be engineered to have increased ability to convert pyruvate to keto-isovalerate. In another embodiment, the yeast microorganism may be engineered to have increased ability to convert pyruvate to 2,3-dihydroxyisovalerate. In another embodiment, the yeast microorganism may be engineered to have increased ability to convert pyruvate to acetolactate.

Furthermore, any of the genes encoding the foregoing enzymes (or any others mentioned herein (or any of the regulatory elements that control or modulate expression thereof)) may be optimized by genetic/protein engineering techniques, such as directed evolution or rational mutagenesis, which are known to those of ordinary skill in the art. Such action allows those of ordinary skill in the art to optimize the enzymes for expression and activity in yeast.

Isobutanol-Producing Metabolic Pathways with Improved KARI Properties

As described herein, the present inventors have discovered that KARI enzymes currently being used in isobutanol-producing recombinant microorganisms suffer from product inhibition (i.e., inhibition by 2,3-dihydroxyisovalerate), resulting in low isobutanol productivity. To overcome this problem and thereby improve isobutanol production, the present inventors have identified a group of KARI enzymes exhibiting reduced inhibition by 2,3-dihydroxyisovalerate. Accordingly, this application describes methods of increasing isobutanol production through the use of recombinant microorganisms comprising KARI enzymes with improved properties for the production of isobutanol.

One aspect of the application is directed to an isolated nucleic acid molecule encoding a ketol-acid reductoisomerase (KARI), wherein said KARI is at least about 60% identical to SEQ ID NO: 2. Further within the scope of present application are KARIs which are at least about 65%, 70%, 75%, 80%, 85%, 90%, 91%, 92%, 93%, 94%, 95%, 96%, 97%, 98%, or 99% identical to SEQ ID NO: 2 and/or SEQ ID NO: 12.

In one embodiment, the KARI is derived from the genus Slackia. In a specific embodiment, the KARI is derived from Slackia exigua. In another specific embodiment, the isolated nucleic acid molecule is comprised of SEQ ID NO: 1. In another embodiment, the KARI is derived from the genus Cryptobacterium. In a specific embodiment, the KARI is derived from Cryptobacterium curtum. In another specific embodiment, the isolated nucleic acid molecule is comprised of SEQ ID NO: 3. In yet another embodiment, the KARI is derived from the genus Eggerthella. In a specific embodiment, the KARI is derived from Eggerthella lenta. In another specific embodiment, the isolated nucleic acid molecule is comprised of SEQ ID NO: 5, SEQ ID NO: 7, or SEQ ID NO: 9. Also included within the scope of this application are isolated KARI enzymes that have been modified to be NADH-dependent. Accordingly, the present application further relates to NADH-dependent ketol-acid reductoisomerases (NKRs) derived from a KARI that is at least about 60% identical to SEQ ID NO: 2 and/or SEQ ID NO: 12.

The invention also includes fragments of the disclosed KARI enzymes which comprise at least 50, 100, 150, 200, 250, 300, 350, 400, 450, 500, 550, or 600 amino acid residues and retain one or more activities associated with KARI enzymes. Such fragments may be obtained by deletion mutation, by recombinant techniques that are routine and well-known in the art, or by enzymatic digestion of the KARI enzyme(s) of interest using any of a number of well-known proteolytic enzymes. The invention further includes nucleic acid molecules which encode the above described mutant KARI enzymes and KARI enzyme fragments.

Another aspect of the application relates to a recombinant microorganism comprising at least one nucleic acid molecule encoding a ketol-acid reductoisomerase (KARI), wherein said KARI is at least about 60% identical to SEQ ID NO: 2 and/or SEQ ID NO: 12. Further within the scope of present application are recombinant microorganism comprising at least one nucleic acid molecule encoding a ketol-acid reductoisomerase (KARI), wherein said KARI is at least about 65%, 70%, 75%, 80%, 85%, 90%, 91%, 92%, 93%, 94%, 95%, 96%, 97%, 98%, or 99% identical to SEQ ID NO: 2.

In one embodiment, the KARI is derived from the genus Slackia. In a specific embodiment, the KARI is derived from Slackia exigua. In another specific embodiment, the KARI is encoded by SEQ ID NO: 1. In another embodiment, the KARI is derived from the genus Cryptobacterium. In a specific embodiment, the KARI is derived from Cryptobacterium curtum. In another specific embodiment, the KARI is encoded by SEQ ID NO: 3. In yet another embodiment, the KARI is derived from the genus Eggerthella. In a specific embodiment, the KARI is derived from Eggerthella lenta. In another specific embodiment, the KARI is encoded by SEQ ID NO: 5, SEQ ID NO: 7, or SEQ ID NO: 9.

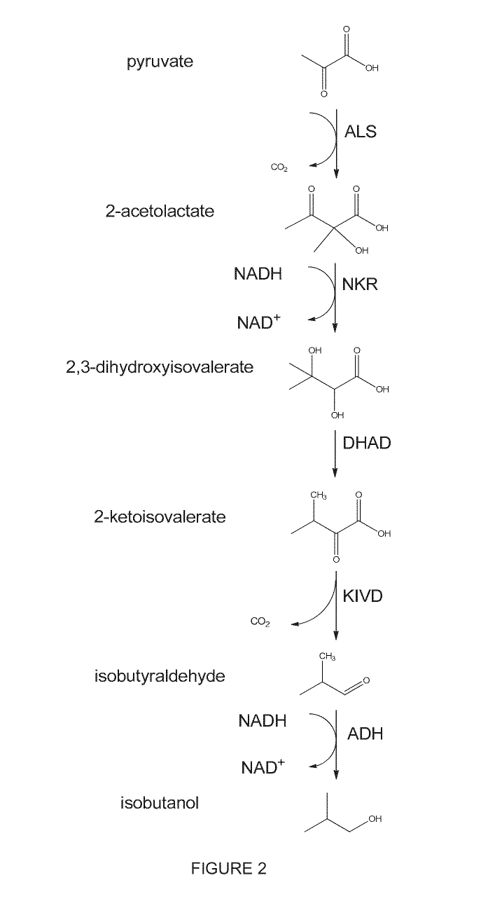

In an exemplary embodiment, pathway steps 2 and 5 of the isobutanol pathway may be carried out by KARI and ADH enzymes that utilize NADH (rather than NADPH) as a cofactor. It has been found previously that utilization of NADH-dependent KARI (NKR) and ADH enzymes to catalyze pathway steps 2 and 5, respectively, surprisingly enables production of isobutanol at theoretical yield and/or under anaerobic conditions. See, e.g., commonly owned and co-pending patent publication US 2010/0143997. An example of an NADH-dependent isobutanol pathway is illustrated in FIG. 2. Thus, in one embodiment, the recombinant microorganisms of the present invention may use an NKR to catalyze the conversion of acetolactate to produce 2,3-dihydroxyisovalerate. In another embodiment, the recombinant microorganisms of the present invention may use an NADH-dependent ADH to catalyze the conversion of isobutyraldehyde to produce isobutanol. In yet another embodiment, the recombinant microorganisms of the present invention may use both an NKR to catalyze the conversion of acetolactate to produce 2,3-dihydroxyisovalerate, and an NADH-dependent ADH to catalyze the conversion of isobutyraldehyde to produce isobutanol.

In an exemplary embodiment, the NKR is derived from a KARI that is at least about 60% identical to SEQ ID NO: 2. In another exemplary embodiment, the NKR is a KARI enzyme that has one or more modifications or mutations at positions corresponding to amino acids selected from: (a) tyrosine 35 of the S. exigua KARI (SEQ ID NO: 2); (b) leucine 57 of the S. exigua KARI (SEQ ID NO: 2); (c) arginine 58 of the S. exigua KARI (SEQ ID NO: 2); (d) glycine 60 of the S. exigua KARI (SEQ ID NO: 2); (e) serine 61 of the S. exigua KARI (SEQ ID NO: 2): (f) serine 62 of the S. exigua KARI (SEQ ID NO: 2); (g) serine 63 of the S. exigua KARI (SEQ ID NO: 2); (h) isoleucine 95 of the S. exigua KARI (SEQ ID NO: 2); and (i) valine 99 of the S. exigua KARI (SEQ ID NO: 2).

In one specific embodiment, the application is directed to KARI enzymes wherein the tyrosine corresponding to position 35 of the S. exigua KARI (SEQ ID NO: 2) is replaced with histidine. In another specific embodiment, the application is directed to KARI enzymes wherein the leucine corresponding to position 57 of the S. exigua KARI (SEQ ID NO: 2) is replaced with serine or arginine. In yet another specific embodiment, the application is directed to KARI enzymes wherein the arginine corresponding to position 58 of the S. exigua KARI (SEQ ID NO: 2) is replaced with proline, alanine, or arginine. In yet another specific embodiment, the application is directed to KARI enzymes wherein the glycine corresponding to position 60 of the S. exigua KARI (SEQ ID NO: 2) is replaced with valine. In yet another specific embodiment, the application is directed to KARI enzymes wherein the serine corresponding to position 61 of the S. exigua KARI (SEQ ID NO: 2) is replaced with aspartic acid, glutamic acid, or cysteine. In yet another specific embodiment, the application is directed to KARI enzymes wherein the serine corresponding to position 62 of the S. exigua KARI (SEQ ID NO: 2) is replaced with proline, alanine, or glutamic acid. In yet another specific embodiment, the application is directed to KARI enzymes wherein the serine corresponding to position 63 of the S. exigua KARI (SEQ ID NO: 2) is replaced with aspartic acid, glutamic acid, histidine, isoleucine, methionine, arginine, or glutamine. In yet another specific embodiment, the application is directed to KARI enzymes wherein the isoleucine corresponding to position 95 of the S. exigua KARI (SEQ ID NO: 2) is replaced with alanine, threonine, valine, or asparagine. In yet another specific embodiment, the application is directed to KARI enzymes wherein the valine corresponding to position 99 of the S. exigua KARI (SEQ ID NO: 2) is replaced with leucine.

In another specific embodiment, the application relates to a KARI enzyme having one or more modifications or mutations at positions corresponding to amino acids selected from: (a) serine 61 of the S. exigua KARI (SEQ ID NO: 2) and (b) serine 63 of the S. exigua KARI (SEQ ID NO: 2).

In another specific embodiment, the application relates to a KARI enzyme having one or more modifications or mutations at positions corresponding to amino acids selected from: (a) serine 63 of the S. exigua KARI (SEQ ID NO: 2) and (b) isoleucine 95 of the S. exigua KARI (SEQ ID NO: 2).

In yet another specific embodiment, the application relates to a KARI enzyme having one or more modifications or mutations at positions corresponding to amino acids selected from: (a) serine 61 of the S. exigua KARI (SEQ ID NO: 2); (b) serine 63 of the S. exigua KARI (SEQ ID NO: 2); and (c) isoleucine 95 of the S. exigua KARI (SEQ ID NO: 2).

In yet another specific embodiment, the application relates to a KARI enzyme having one or more modifications or mutations at positions corresponding to amino acids selected from (a) leucine 57 of the S. exigua KARI (SEQ ID NO: 2); (b) arginine 58 of the S. exigua KARI (SEQ ID NO: 2); and (c) serine 63 of the S. exigua KARI (SEQ ID NO: 2).

Further included within the scope of the application are KARI enzymes, other than the S. exigua KARI (SEQ ID NO: 2), which contain modifications or mutations corresponding to those set out above. The nucleotide sequences for several KARI enzymes are known. A representative listing of KARI enzymes capable of being modified are disclosed in commonly owned and co-pending US Publication No. US 2010/0143997.

The corresponding positions of the KARI enzyme identified herein (e.g., the S. exigua KARI) may be readily identified for other KARI enzymes by one of skill in the art. Thus, given the defined region and the assays described in the present application, one with skill in the art can make one or a number of modifications which would result in an increased ability to utilize NADH, particularly for the conversion of acetolactate to 2,3-dihydroxyisovalerate, in any KARI enzyme of interest. Residues to be modified in accordance with the present application may include those described in Examples 6-9.

The application also includes fragments of the modified KARI enzymes which comprise at least 50, 100, 150, 200, 250, 300, 350, 400, 450, 500, 550, or 600 amino acid residues and retain one or more activities associated with KARI enzymes. Such fragments may be obtained by deletion mutation, by recombinant techniques that are routine and well-known in the art, or by enzymatic digestion of the KARI enzyme(s) of interest using any of a number of well-known proteolytic enzymes. The invention further includes nucleic acid molecules which encode the above described mutant KARI enzymes and KARI enzyme fragments.

Another aspect of the application relates to a recombinant microorganism comprising at least one nucleic acid molecule encoding a ketol-acid reductoisomerase (KARI), wherein said KARI has one or more modifications or mutations at positions corresponding to amino acids selected from: (a) tyrosine 35 of the S. exigua KARI (SEQ ID NO: 2); (b) leucine 57 of the S. exigua KARI (SEQ ID NO: 2); (c) arginine 58 of the S. exigua KARI (SEQ ID NO: 2); (d) glycine 60 of the S. exigua KARI (SEQ ID NO: 2); (e) serine 61 of the S. exigua KARI (SEQ ID NO: 2); (f) serine 62 of the S. exigua KARI (SEQ ID NO: 2); (g) serine 63 of the S. exigua KARI (SEQ ID NO: 2); (h) isoleucine 95 of the S. exigua KARI (SEQ ID NO: 2); and (i) valine 99 of the S. exigua KARI (SEQ ID NO: 2). Further included within the scope of the application are recombinant microorganisms comprising a KARI enzyme, other than the S. exigua KARI (SEQ ID NO: 2), which contains modifications or mutations at positions corresponding to those set out above.

Further within the scope of present application are recombinant microorganisms comprising at least one nucleic acid molecule encoding a ketol-acid reductoisomerase (KARI), wherein said KARI is at least about 65%, 70%, 75%, 80%, 85%, 90%, 91%, 92%, 93%, 94%, 95%, 96%, 97%, 98%, or 99% identical to SEQ ID NO: 2. Also within the scope of present application are recombinant microorganisms comprising at least one nucleic acid molecule encoding a ketol-acid reductoisomerase (KARI), wherein said KARI is at least about 65%, 70%, 75%, 80%, 85%, 90%, 91%, 92%, 93%, 94%, 95%, 96%, 97%, 98%, or 99% identical to a KARI having one or more modifications or mutations at positions corresponding to amino acids selected from: (a) tyrosine 35 of the S. exigua KARI (SEQ ID NO: 2); (b) leucine 57 of the S. exigua KARI (SEQ ID NO: 2); (c) arginine 58 of the S. exigua KARI (SEQ ID NO: 2); (d) glycine 60 of the S. exigua KARI (SEQ ID NO: 2); (e) serine 61 of the S. exigua KARI (SEQ ID NO: 2); (f) serine 62 of the S. exigua KARI (SEQ ID NO: 2); (g) serine 63 of the S. exigua KARI (SEQ ID NO: 2); (h) isoleucine 95 of the S. exigua KARI (SEQ ID NO: 2); and (i) valine 99 of the S. exigua KARI (SEQ ID NO: 2).

In accordance with the invention, any number of mutations can be made to the KARI enzymes, and in a preferred aspect, multiple mutations can be made to result in an increased ability to utilize NADH for the conversion of acetolactate to 2,3-dihydroxyisovalerate. Such mutations include point mutations, frame shift mutations, deletions, and insertions, with one or more (e.g., one, two, three, four, five or more, etc.) point mutations preferred.

Mutations may be introduced into the KARI enzymes of the present application to create NKRs using any methodology known to those skilled in the art. Mutations may be introduced randomly by, for example, conducting a PCR reaction in the presence of manganese as a divalent metal ion cofactor. Alternatively, oligonucleotide directed mutagenesis may be used to create the NKRs which allows for all possible classes of base pair changes at any determined site along the encoding DNA molecule. In general, this technique involves annealing an oligonucleotide complementary (except for one or more mismatches) to a single stranded nucleotide sequence coding for the KARI enzyme of interest. The mismatched oligonucleotide is then extended by DNA polymerase, generating a double-stranded DNA molecule which contains the desired change in sequence in one strand. The changes in sequence can, for example, result in the deletion, substitution, or insertion of an amino acid. The double-stranded polynucleotide can then be inserted into an appropriate expression vector, and a mutant or modified polypeptide can thus be produced. The above-described oligonucleotide directed mutagenesis can, for example, be carried out via PCR.

In one aspect, the NADH-dependent activity of the modified or mutated KARI enzyme is increased.

In an exemplary embodiment, the catalytic efficiency of the modified or mutated KARI enzyme is improved for the cofactor NADH. Preferably, the catalytic efficiency of the modified or mutated KARI enzyme is improved by at least about 5% as compared to the wild-type or parental KARI for NADH. More preferably the catalytic efficiency of the modified or mutated KARI enzyme is improved by at least about 15% as compared to the wild-type or parental KARI for NADH. More preferably, the catalytic efficiency of the modified or mutated KARI enzyme is improved by at least about 25% as compared to the wild-type or parental KARI for NADH. More preferably, the catalytic efficiency of the modified or mutated KARI enzyme is improved by at least about 50% as compared to the wild-type or parental KARI for NADH. More preferably, the catalytic efficiency of the modified or mutated KARI enzyme is improved by at least about 75% as compared to the wild-type or parental KARI for NADH. More preferably, the catalytic efficiency of the modified or mutated KARI enzyme is improved by at least about 100% as compared to the wild-type or parental KARI for NADH. More preferably, the catalytic efficiency of the modified or mutated KARI enzyme is improved by at least about 300% as compared to the wild-type or parental KARI for NADH. More preferably, the catalytic efficiency of the modified or mutated KARI enzyme is improved by at least about 500% as compared to the wild-type or parental KARI for NADH. More preferably, the catalytic efficiency of the modified or mutated KARI enzyme is improved by at least about 1000% as compared to the wild-type or parental KARI for NADH. More preferably, the catalytic efficiency of the modified or mutated KARI enzyme is improved by at least about 5000% as compared to the wild-type or parental KARI for NADH.

In another exemplary embodiment, the catalytic efficiency of the modified or mutated KARI enzyme with NADH is increased with respect to the catalytic efficiency of the wild-type or parental enzyme with NADPH. Preferably, the catalytic efficiency of the modified or mutated KARI enzyme is at least about 10% of the catalytic efficiency of the wild-type or parental KARI enzyme for NADPH. More preferably, the catalytic efficiency of the modified or mutated KARI enzyme is at least about 25% of the catalytic efficiency of the wild-type or parental KARI enzyme for NADPH. More preferably, the catalytic efficiency of the modified or mutated KARI enzyme is at least about 50% of the catalytic efficiency of the wild-type or parental KARI enzyme for NADPH. More preferably, the catalytic efficiency of the modified or mutated KARI enzyme is at least about 75%, 85%, 95% of the catalytic efficiency of the wild-type or parental KARI enzyme for NADPH.

In another exemplary embodiment, the K_{M} of the KARI enzyme for NADH is decreased relative to the wild-type or parental enzyme. A change in K_{M} is evidenced by at least a 5% or greater increase or decrease in K_{M} compared to the wild-type KARI enzyme. In certain embodiments, modified or mutated KARI enzymes of the present invention may show greater than 10 times decreased K_{M} for NADH compared to the wild-type or parental KARI enzyme. In certain embodiments, modified or mutated KARI enzymes of the present invention may show greater than 30 times decreased K_{M} for NADH compared to the wild-type or parental KARI enzyme.

In another exemplary embodiment, the k_{cat} of the KARI enzyme with NADH is increased relative to the wild-type or parental enzyme. A change in k_{cat} is evidenced by at least a 5% or greater increase or decrease in K_{M} compared to the wild-type KARI enzyme. In certain embodiments, modified or mutated KARI enzymes of the present invention may show greater than 50% increased k_{cat} for NADH compared to the wild-type or parental KARI enzyme. In certain embodiments, modified or mutated KARI enzymes of the present invention may show greater than 100% increased k_{cat} for NADH compared to the wild-type or parental KARI enzyme. In certain embodiments, modified or mutated KARI enzymes of the present invention may show greater than 200% increased k_{cat} for NADH compared to the wild-type or parental KARI enzyme.

Recombinant Microorganisms Comprising KARI with Improved Properties

In addition to isobutanol producing metabolic pathways, a number of biosynthetic pathways use KARI enzymes to catalyze a reaction step, including pathways for the production of isoleucine, leucine, valine, pantothenate, coenzyme A, 1-butanol, 2-methyl-1-butanol, 3-methyl-1-butanol, 3-methyl-1-pentanol, 4-methyl-1-pentanol, 4-methyl-1-hexanol, and 5-methyl-1-heptanol. A representative list of the engineered biosynthetic pathways utilizing KARI enzymes is provided in Table 1.

| Biosynthetic Pathway | Reference^{a} |
|---|---|
| Isobutanol | US 2009/0226991 (Feldman et al.), US 2011/0020889 (Feldman et |
| | al.), and US 2010/0143997 (Buelter et al.) |
| Leucine | WO/2001/021772 (Yocum et al.) and McCourt et al., 2006, Amino |
| | Acids 31: 173-210 |
| Valine | WO/2001/021772 (Yocum et al.) and McCourt et al., 2006, Amino |
| | Acids 31: 173-210 |
| Pantothenic Acid | WO/2001/021772 (Yocum et al.) |
| Coenzyme A | WO/2001/021772 (Yocum et al.) |
| 1-Butanol | WO/2010/017230 (Lynch), WO/2010/031772 (Wu et al.), and |
| | KR2011002130 (Lee et al.) |
| 2-Methyl-1-Butanol | WO/2008/098227 (Liao et al.), WO/2009/076480 (Picataggio et al.), |
| | and Atsumi et al., 2008, Nature 451: 86-89 |
| 3-Methyl-1-Butanol | WO/2008/098227 (Liao et al.), Atsumi et al., 2008. Nature 451: 86- |
| | 89, and Connor et al., 2008, Appl. Environ. Microbiol. 74: 5769-5775 |
| 3-Methyl-1-Pentanol | WO/2010/045629 (Liao et al.), Zhang et al., 2008, Proc Natl Acad |
| | Sci USA 105: 20653-20658 |
| 4-Methyl-1-Pentanol | WO/2010/045629 (Liao et al.), Zhang et al., 2008, Proc Natl Acad |
| | Sci USA 105: 20653-20658 |
| 4-Methyl-1-Hexanol | WO/2010/045629 (Liao at el.), Zhang et al., 2008, Proc Natl Acad |
| | Sci USA 105: 20653-20658 |
| 5-Methyl-1-Heptanol | WO/2010/045629 (Liao et al.), Zhang et al., 2008, Proc Natl Acad |
| | Sci USA 105: 20653-20658 |

^{a}The contents of each of the references in this table are herein incorporated by reference in their entireties for all purposes.

As described above, each of these biosynthetic pathways uses a KARI enzyme to catalyze a reaction step. Therefore, the product yield from these biosynthetic pathways will in part depend upon the activity of KARI.

As will be understood by one skilled in the art equipped with the present disclosure, the KARI enzymes described herein would have utility in any of the above-described pathways. Thus, in an additional aspect, the present application relates to a recombinant microorganism comprising a KARI-requiring biosynthetic pathway, wherein said recombinant microorganism comprises at least one nucleic acid molecule encoding a KARI that is at least about 60% identical to SEQ ID NO: 2 and/or SEQ ID NO: 12. In one embodiment, the KARI is derived from the genus Slackia. In a specific embodiment, the KARI is derived from Slackia exigua. In another specific embodiment, the KARI is encoded by SEQ ID NO: 1. In another embodiment, the KARI is derived from the genus Cryptobacterium. In a specific embodiment, the KARI is derived from Cryptobacterium curtum. In another specific embodiment, the KARI is encoded by SEQ ID NO: 3. In yet another embodiment, the KARI is derived from the genus Eggerthella. In a specific embodiment, the KARI is derived from Eggerthella lenta. In another specific embodiment, the KARI is encoded by SEQ ID NO: 5, SEQ ID NO: 7, or SEQ ID NO: 9. In yet another embodiment, the KARI has one or more modifications or mutations at positions corresponding to amino acids selected from: (a) tyrosine 35 of the S. exigua KARI (SEQ ID NO: 2); (b) leucine 57 of the S. exigua KARI (SEQ ID NO: 2); (c) arginine 58 of the S. exigua KARI (SEQ ID NO: 2); (d) glycine 60 of the S. exigua KARI (SEQ ID NO: 2); (e) serine 61 of the S. exigua KARI (SEQ ID NO: 2); (f) serine 62 of the S. exigua KARI (SEQ ID NO: 2); (g) serine 63 of the S. exigua KARI (SEQ ID NO: 2); (h) isoleucine 95 of the S. exigua KARI (SEQ ID NO: 2); and (i) valine 99 of the S. exigua KARI (SEQ ID NO: 2).

As used herein, a “KARI-requiring biosynthetic pathway” refers to any metabolic pathway which utilizes KARI to convert acetolactate to 2,3-dihydroxyisovalerate or 2-aceto-2-hydroxy-butanoate to 2,3-dihydroxy-3-methylvalerate. Examples of KARI-requiring biosynthetic pathways include, but are not limited to, isobutanol, isoleucine, leucine, valine, pantothenate, coenzyme A, 1-butanol, 2-methyl-1-butanol, 3-methyl-1-butanol, 3-methyl-1-pentanol, 4-methyl-1-pentanol, 4-methyl-1-hexanol, and 5-methyl-1-heptanol metabolic pathways. The metabolic pathway may naturally occur in a microorganism (e.g., a natural pathway for the production of valine) or arise from the introduction of one or more heterologous polynucleotides through genetic engineering. In an exemplary embodiment, the recombinant microorganisms expressing the KARI-requiring biosynthetic pathway are yeast cells.

### The Microorganism in General

As described herein, the recombinant microorganisms of the present invention can express a plurality of heterologous and/or native enzymes involved in pathways for the production of a beneficial metabolite such as isobutanol.

As described herein, “engineered” or “modified” microorganisms are produced via the introduction of genetic material into a host or parental microorganism of choice and/or by modification of the expression of native genes, thereby modifying or altering the cellular physiology and biochemistry of the microorganism. Through the introduction of genetic material and/or the modification of the expression of native genes the parental microorganism acquires new properties, e.g., the ability to produce a new, or greater quantities of, an intracellular and/or extracellular metabolite. As described herein, the introduction of genetic material into and/or the modification of the expression of native genes in a parental microorganism results in a new or modified ability to produce beneficial metabolites such as isobutanol from a suitable carbon source. The genetic material introduced into and/or the genes modified for expression in the parental microorganism contains gene(s), or parts of genes, coding for one or more of the enzymes involved in a biosynthetic pathway for the production of isobutanol and may also include additional elements for the expression and/or regulation of expression of these genes, e.g., promoter sequences.

In addition to the introduction of a genetic material into a host or parental microorganism, an engineered or modified microorganism can also include the alteration, disruption, deletion or knocking-out of a gene or polynucleotide to alter the cellular physiology and biochemistry of the microorganism. Through the alteration, disruption, deletion or knocking-out of a gene or polynucleotide, the microorganism acquires new or improved properties (e.g., the ability to produce a new metabolite or greater quantities of an intracellular metabolite, to improve the flux of a metabolite down a desired pathway, and/or to reduce the production of by-products).

Recombinant microorganisms provided herein may also produce metabolites in quantities not available in the parental microorganism. A “metabolite” refers to any substance produced by metabolism or a substance necessary for or taking part in a particular metabolic process. A metabolite can be an organic compound that is a starting material (e.g., glucose or pyruvate), an intermediate (e.g., 2-ketoisovalerate), or an end product (e.g., isobutanol) of metabolism. Metabolites can be used to construct more complex molecules, or they can be broken down into simpler ones. Intermediate metabolites may be synthesized from other metabolites, perhaps used to make more complex substances, or broken down into simpler compounds, often with the release of chemical energy.

The disclosure identifies specific genes useful in the methods, compositions and organisms of the disclosure; however it will be recognized that absolute identity to such genes is not necessary. For example, changes in a particular gene or polynucleotide comprising a sequence encoding a polypeptide or enzyme can be performed and screened for activity. Typically such changes comprise conservative mutations and silent mutations. Such modified or mutated polynucleotides and polypeptides can be screened for expression of a functional enzyme using methods known in the art.

Due to the inherent degeneracy of the genetic code, other polynucleotides which encode substantially the same or functionally equivalent polypeptides can also be used to clone and express the polynucleotides encoding such enzymes.

As will be understood by those of skill in the art, it can be advantageous to modify a coding sequence to enhance its expression in a particular host. The genetic code is redundant with 64 possible codons, but most organisms typically use a subset of these codons. The codons that are utilized most often in a species are called optimal codons, and those not utilized very often are classified as rare or low-usage codons. Codons can be substituted to reflect the preferred codon usage of the host, in a process sometimes called “codon optimization” or “controlling for species codon bias.”

Optimized coding sequences containing codons preferred by a particular prokaryotic or eukaryotic host (Murray et al., 1989, Nucl Acids Res. 17: 477-508) can be prepared, for example, to increase the rate of translation or to produce recombinant RNA transcripts having desirable properties, such as a longer half-life, as compared with transcripts produced from a non-optimized sequence. Translation stop codons can also be modified to reflect host preference. For example, typical stop codons for S. cerevisiae and mammals are UAA and UGA, respectively. The typical stop codon for monocotyledonous plants is UGA, whereas insects and E. coli commonly use UAA as the stop codon (Dalphin et al., 1996, Nucl Acids Res. 24: 216-8). Methodology for optimizing a nucleotide sequence for expression in a plant is provided, for example, in U.S. Pat. No. 6,015,891, and the references cited therein.

Those of skill in the art will recognize that, due to the degenerate nature of the genetic code, a variety of DNA compounds differing in their nucleotide sequences can be used to encode a given enzyme of the disclosure. The native DNA sequence encoding the biosynthetic enzymes described above are referenced herein merely to illustrate an embodiment of the disclosure, and the disclosure includes DNA compounds of any sequence that encode the amino acid sequences of the polypeptides and proteins of the enzymes utilized in the methods of the disclosure. In similar fashion, a polypeptide can typically tolerate one or more amino acid substitutions, deletions, and insertions in its amino acid sequence without loss or significant loss of a desired activity. The disclosure includes such polypeptides with different amino acid sequences than the specific proteins described herein so long as the modified or variant polypeptides have the enzymatic anabolic or catabolic activity of the reference polypeptide. Furthermore, the amino acid sequences encoded by the DNA sequences shown herein merely illustrate embodiments of the disclosure.

In addition, homologs of enzymes useful for generating metabolites are encompassed by the microorganisms and methods provided herein.

As used herein, two proteins (or a region of the proteins) are substantially homologous when the amino acid sequences have at least about 30%, 40%, 50% 60%, 65%, 70%, 75%, 80%, 85%, 90%, 91%, 92%, 93%, 94%, 95%, 96%, 97%, 98%, or 99% identity. To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). In one embodiment, the length of a reference sequence aligned for comparison purposes is at least 30%, typically at least 40%, more typically at least 50%, even more typically at least 60%, and even more typically at least 70%, 80%, 90%, 100% of the length of the reference sequence. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology”). The percent identity between the two sequences is a function of the number of identical positions shared by the sequences, taking into account the number of gaps, and the length of each gap, which need to be introduced for optimal alignment of the two sequences.

When “homologous” is used in reference to proteins or peptides, it is recognized that residue positions that are not identical often differ by conservative amino acid substitutions. A “conservative amino acid substitution” is one in which an amino acid residue is substituted by another amino acid residue having a side chain (R group) with similar chemical properties (e.g., charge or hydrophobicity). In general, a conservative amino acid substitution will not substantially change the functional properties of a protein. In cases where two or more amino acid sequences differ from each other by conservative substitutions, the percent sequence identity or degree of homology may be adjusted upwards to correct for the conservative nature of the substitution. Means for making this adjustment are well known to those of skill in the art (See, e.g., Pearson W. R., 1994, Methods in Mol Biol 25: 365-89).

The following six groups each contain amino acids that are conservative substitutions for one another: 1) Serine (S), Threonine (T); 2) Aspartic Acid (D), Glutamic Acid (E); 3) Asparagine (N), Glutamine (Q); 4) Arginine (R), Lysine (K); 5) Isoleucine (I). Leucine (L), Alanine (A), Valine (V), and 6) Phenylalanine (F), Tyrosine (Y), Tryptophan (W).

Sequence homology for polypeptides, which is also referred to as percent sequence identity, is typically measured using sequence analysis software. See commonly owned and co-pending application US 2009/0226991. A typical algorithm used comparing a molecule sequence to a database containing a large number of sequences from different organisms is the computer program BLAST. When searching a database containing sequences from a large number of different organisms, it is typical to compare amino acid sequences. Database searching using amino acid sequences can be measured by algorithms described in commonly owned U.S. Pat. No. 8,017,375.

It is understood that a range of microorganisms can be modified to include an isobutanol producing metabolic pathway suitable for the production of isobutanol. In various embodiments, the microorganisms may be selected from yeast microorganisms. Yeast microorganisms for the production of isobutanol may be selected based on certain characteristics:

One characteristic may include the property that the microorganism is selected to convert various carbon sources into isobutanol. The term “carbon source” generally refers to a substance suitable to be used as a source of carbon for prokaryotic or eukaryotic cell growth. Examples of suitable carbon sources are described in commonly owned U.S. Pat. No. 8,017,375. Accordingly, in one embodiment, the recombinant microorganism herein disclosed can convert a variety of carbon sources to products, including but not limited to glucose, galactose, mannose, xylose, arabinose, lactose, sucrose, CO₂, and mixtures thereof.

The recombinant microorganism may thus further include a pathway for the production of isobutanol from five-carbon (pentose) sugars including xylose. Most yeast species metabolize xylose via a complex route, in which xylose is first reduced to xylitol via a xylose reductase (XR) enzyme. The xylitol is then oxidized to xylulose via a xylitol dehydrogenase (XDH) enzyme. The xylulose is then phosphorylated via a xylulokinase (XK) enzyme. This pathway operates inefficiently in yeast species because it introduces a redox imbalance in the cell. The xylose-to-xylitol step uses primarily NADPH as a cofactor (generating NADP+), whereas the xylitol-to-xylulose step uses NAD+ as a cofactor (generating NADH). Other processes must operate to restore the redox imbalance within the cell. This often means that the organism cannot grow anaerobically on xylose or other pentose sugars. Accordingly, a yeast species that can efficiently ferment xylose and other pentose sugars into a desired fermentation product is therefore very desirable.

Thus, in one aspect, the recombinant microorganism is engineered to express a functional exogenous xylose isomerase. Exogenous xylose isomerases (XI) functional in yeast are known in the art. See, e.g., Rajgarhia et al., U.S. Pat. No. 7,943,366, which is herein incorporated by reference in its entirety. In an embodiment according to this aspect, the exogenous XI gene is operatively linked to promoter and terminator sequences that are functional in the yeast cell. In a preferred embodiment, the recombinant microorganism further has a deletion or disruption of a native gene that encodes for an enzyme (e.g., XR and/or XDH) that catalyzes the conversion of xylose to xylitol. In a further preferred embodiment, the recombinant microorganism also contains a functional, exogenous xylulokinase (XK) gene operatively linked to promoter and terminator sequences that are functional in the yeast cell. In one embodiment, the xylulokinase (XK) gene is overexpressed.

In one embodiment, the yeast microorganism has reduced or no pyruvate decarboxylase (PDC) activity. PDC catalyzes the decarboxylation of pyruvate to acetaldehyde, which is then reduced to ethanol by ADH via an oxidation of NADH to NAD+. Ethanol production is the main pathway to oxidize the NADH from glycolysis. Deletion, disruption, or mutation of this pathway increases the pyruvate and the reducing equivalents (NADH) available for a biosynthetic pathway which uses pyruvate as the starting material and/or as an intermediate. Accordingly, deletion, disruption, or mutation of one or more genes encoding for pyruvate decarboxylase and/or a positive transcriptional regulator thereof can further increase the yield of the desired pyruvate-derived metabolite (e.g., isobutanol). In one embodiment, said pyruvate decarboxylase gene targeted for disruption, deletion, or mutation is selected from the group consisting of PDC1, PDC5, and PDC6, or homologs or variants thereof. In another embodiment, all three of PDC1, PDC5, and PDC6 are targeted for disruption, deletion, or mutation. In yet another embodiment, a positive transcriptional regulator of the PDC1, PDC5, and/or PDC6 is targeted for disruption, deletion or mutation. In one embodiment, said positive transcriptional regulator is PDC2, or homologs or variants thereof.

As is understood by those skilled in the art, there are several additional mechanisms available for reducing or disrupting the activity of a protein encoded by PDC1. PDC5, PDC6, and/or PDC2, including, but not limited to, the use of a regulated promoter, use of a weak constitutive promoter, disruption of one of the two copies of the gene in a diploid yeast, disruption of both copies of the gene in a diploid yeast, expression of an anti-sense nucleic acid, expression of an siRNA, over expression of a negative regulator of the endogenous promoter, alteration of the activity of an endogenous or heterologous gene, use of a heterologous gene with lower specific activity, the like or combinations thereof. Yeast strains with reduced PDC activity are described in commonly owned U.S. Pat. No. 8,017,375, as well as commonly owned and co-pending US Patent Publication No. 2011/0183392.

In another embodiment, the microorganism has reduced glycerol-3-phosphate dehydrogenase (GPD) activity. GPD catalyzes the reduction of dihydroxyacetone phosphate (DHAP) to glycerol-3-phosphate (G3P) via the oxidation of NADH to NAD+. Glycerol is then produced from G3P by Glycerol-3-phosphatase (GPP). Glycerol production is a secondary pathway to oxidize excess NADH from glycolysis. Reduction or elimination of this pathway would increase the pyruvate and reducing equivalents (NADH) available for the production of a pyruvate-derived metabolite (e.g., isobutanol). Thus, disruption, deletion, or mutation of the genes encoding for glycerol-3-phosphate dehydrogenases can further increase the yield of the desired metabolite (e.g., isobutanol). Yeast strains with reduced GPD activity are described in commonly owned and co-pending US Patent Publication Nos. 2011/0020889 and 2011/0183392.

In yet another embodiment, the microorganism has reduced 3-keto acid reductase (3-KAR) activity. 3-KARs catalyze the conversion of 3-keto acids (e.g., acetolactate) to 3-hydroxyacids (e.g., DH2 MB). Yeast strains with reduced 3-KAR activity are described in commonly owned U.S. Pat. Nos. 8,133,715, 8,153,415, and 8,158,404, which are herein incorporated by reference in their entireties.

In yet another embodiment, the microorganism has reduced aldehyde dehydrogenase (ALDH) activity. Aldehyde dehydrogenases catalyze the conversion of aldehydes (e.g., isobutyraldehyde) to acid by-products (e.g., isobutyrate). Yeast strains with reduced ALDH activity are described in commonly owned U.S. Pat. Nos. 8,133,715, 8,153,415, and 8,158,404, which are herein incorporated by reference in their entireties.

In one embodiment, the yeast microorganisms may be selected from the “Saccharomyces Yeast Clade”, as described in commonly owned U.S. Pat. No. 8,017,375.

The term “Saccharomyces sensu stricto” taxonomy group is a cluster of yeast species that are highly related to S. cerevisiae (Rainieri et al., 2003, J. Biosci Bioengin 96: 1-9). Saccharomyces sensu stricto yeast species include but are not limited to S. cerevisiae, S. kudriavzevii, S. mikatae, S. bayanus, S. uvarum, S. carocanis and hybrids derived from these species (Masneuf et al., 1998, Yeast 7: 61-72).

An ancient whole genome duplication (WGD) event occurred during the evolution of the hemiascomycete yeast and was discovered using comparative genomic tools (Kellis et al., 2004, Nature 428: 617-24; Dujon et al., 2004, Nature 430:35-44; Langkjaer et al., 2003, Nature 428: 848-52; Wolfe et al., 1997, Nature 387: 708-13). Using this major evolutionary event, yeast can be divided into species that diverged from a common ancestor following the WGD event (termed “post-WGD yeast” herein) and species that diverged from the yeast lineage prior to the WGD event (termed “pre-WGD yeast” herein).

Accordingly, in one embodiment, the yeast microorganism may be selected from a post-WGD yeast genus, including but not limited to Saccharomyces and Candida. The favored post-WGD yeast species include: S. cerevisiae, S. uvarum, S. bayanus, S. paradoxus, S. castelli, and C. glabrata.

In another embodiment, the yeast microorganism may be selected from a pre-whole genome duplication (pre-WGD) yeast genus including but not limited to Saccharomyces, Kluyveromyces, Candida, Pichia, Issatchenkia, Debaryomyces, Hansenula, Yarrowia and, Schizosaccharomyces. Representative pre-WGD yeast species include: S. kluyveri, K. thermotolerans, K. marxianus, K. waltii, K. lactis, C. tropicalis, P. pastoris, P. anomala, P. stipitis, I. orientalis, I. occidentalis, I. scutulata, D. hansenii, H. anomala, Y. lipolytica, and S. pombe.

A yeast microorganism may be either Crabtree-negative or Crabtree-positive as described in described in commonly owned U.S. Pat. No. 8,017,375. In one embodiment the yeast microorganism may be selected from yeast with a Crabtree-negative phenotype including but not limited to the following genera: Saccharomyces, Kluyveromyces, Pichia, Issatchenkia, Hansenula, and Candida. Crabtree-negative species include but are not limited to: S. kluyveri, K. lactis, K. marxianus, P. anomala, P. stipitis, I. orientalis, I. occidentalis, I. scutulata, H. anomala, and C. utilis. In another embodiment, the yeast microorganism may be selected from yeast with a Crabtree-positive phenotype, including but not limited to Saccharomyces, Kluyveromyces, Zygosaccharomyces, Debaryomyces, Pichia and Schizosaccharomyces. Crabtree-positive yeast species include but are not limited to: S. cerevisiae, S. uvarum, S. bayanus, S. paradoxus, S. castelli, K. thermotolerans, C. glabrata, Z. bailli, Z. rouxii, D. hansenii, P. pastorius, and S. pombe.

Another characteristic may include the property that the microorganism is that it is non-fermenting. In other words, it cannot metabolize a carbon source anaerobically while the yeast is able to metabolize a carbon source in the presence of oxygen. Nonfermenting yeast refers to both naturally occurring yeasts as well as genetically modified yeast. During anaerobic fermentation with fermentative yeast, the main pathway to oxidize the NADH from glycolysis is through the production of ethanol. Ethanol is produced by alcohol dehydrogenase (ADH) via the reduction of acetaldehyde, which is generated from pyruvate by pyruvate decarboxylase (PDC). In one embodiment, a fermentative yeast can be engineered to be non-fermentative by the reduction or elimination of the native PDC activity. Thus, most of the pyruvate produced by glycolysis is not consumed by PDC and is available for the isobutanol pathway. Deletion of this pathway increases the pyruvate and the reducing equivalents available for the biosynthetic pathway. Fermentative pathways contribute to low yield and low productivity of pyruvate-derived metabolites such as isobutanol. Accordingly, deletion of one or more PDC genes may increase yield and productivity of a desired metabolite (e.g., isobutanol).

In some embodiments, the recombinant microorganisms may be microorganisms that are non-fermenting yeast microorganisms, including, but not limited to those, classified into a genera selected from the group consisting of Tricosporon, Rhodotorula, Myxozyma, or Candida. In a specific embodiment, the non-fermenting yeast is C. xestobii.

### Methods in General

### Identification of KARI Homologs

Any method can be used to identify genes that encode for enzymes that are homologous to the genes described herein (e.g., KARI homologs). Generally, genes that are homologous or similar to the KARIs described herein may be identified by functional, structural, and/or genetic analysis. In most cases, homologous or similar genes and/or homologous or similar enzymes will have functional, structural, or genetic similarities.

Techniques known to those skilled in the art may be suitable to identify additional homologous genes and homologous enzymes. Generally, analogous genes and/or analogous enzymes can be identified by functional analysis and will have functional similarities. Techniques known to those skilled in the art may be suitable to identify analogous genes and analogous enzymes. For example, to identify homologous or analogous genes, proteins, or enzymes, techniques may include, but not limited to, cloning a gene by PCR using primers based on a published sequence of a gene/enzyme or by degenerate PCR using degenerate primers designed to amplify a conserved region among ketol-acid reductoisomerase genes. Further, one skilled in the art can use techniques to identify homologous or analogous genes, proteins, or enzymes with functional homology or similarity. Techniques include examining a cell or cell culture for the catalytic activity of an enzyme through in vitro enzyme assays for said activity (e.g. as described herein or in Kiritani, K. Branched-Chain Amino Acids Methods Enzymology, 1970), then isolating the enzyme with said activity through purification, determining the protein sequence of the enzyme through techniques such as Edman degradation, design of PCR primers to the likely nucleic acid sequence, amplification of said DNA sequence through PCR, and cloning of said nucleic acid sequence. To identify homologous or similar genes and/or homologous or similar enzymes, analogous genes and/or analogous enzymes or proteins, techniques also include comparison of data concerning a candidate gene or enzyme with databases such as BRENDA, KEGG, or MetaCYC. The candidate gene or enzyme may be identified within the above mentioned databases in accordance with the teachings herein.

### Genetic Insertions and Deletions

Any method can be used to introduce a nucleic acid molecule into yeast and many such methods are well known. For example, transformation and electroporation are common methods for introducing nucleic acid into yeast cells. See, e.g., Gietz et al., 1992, Nuc Acids Res. 27: 69-74; Ito et al., 1983, J. Bacteriol. 153: 163-8; and Becker et al., 1991, Methods in Enzymology 194: 182-7.

In an embodiment, the integration of a gene of interest into a DNA fragment or target gene of a yeast microorganism occurs according to the principle of homologous recombination. According to this embodiment, an integration cassette containing a module comprising at least one yeast marker gene and/or the gene to be integrated (internal module) is flanked on either side by DNA fragments homologous to those of the ends of the targeted integration site (recombinogenic sequences). After transforming the yeast with the cassette by appropriate methods, a homologous recombination between the recombinogenic sequences may result in the internal module replacing the chromosomal region in between the two sites of the genome corresponding to the recombinogenic sequences of the integration cassette. (Orr-Weaver et al., 1981, PNAS USA 78: 6354-58).

In an embodiment, the integration cassette for integration of a gene of interest into a yeast microorganism includes the heterologous gene under the control of an appropriate promoter and terminator together with the selectable marker flanked by recombinogenic sequences for integration of a heterologous gene into the yeast chromosome. In an embodiment, the heterologous gene includes an appropriate native gene desired to increase the copy number of a native gene(s). The selectable marker gene can be any marker gene used in yeast, including but not limited to, HIS3, TRP1, LEU2, URA3, bar, ble, hph, and kan. The recombinogenic sequences can be chosen at will, depending on the desired integration site suitable for the desired application.

In another embodiment, integration of a gene into the chromosome of the yeast microorganism may occur via random integration (Kooistra et al., 2004, Yeast 21: 781-792).

Additionally, in an embodiment, certain introduced marker genes are removed from the genome using techniques well known to those skilled in the art. For example, URA3 marker loss can be obtained by plating URA3 containing cells in FOA (5-fluoro-orotic acid) containing medium and selecting for FOA resistant colonies (Boeke et al., 1984, Mol. Gen. Genet 197: 345-47).

The exogenous nucleic acid molecule contained within a yeast cell of the disclosure can be maintained within that cell in any form. For example, exogenous nucleic acid molecules can be integrated into the genome of the cell or maintained in an episomal state that can stably be passed on (“inherited”) to daughter cells. Such extra-chromosomal genetic elements (such as plasmids, mitochondrial genome, etc.) can additionally contain selection markers that ensure the presence of such genetic elements in daughter cells. Moreover, the yeast cells can be stably or transiently transformed. In addition, the yeast cells described herein can contain a single copy, or multiple copies of a particular exogenous nucleic acid molecule as described above.

### Reduction of Enzymatic Activity

Yeast microorganisms within the scope of the invention may have reduced enzymatic activity such as reduced PDC, GPD, ALDH, or 3-KAR activity. The term “reduced” as used herein with respect to a particular polypeptide activity refers to a lower level of polypeptide activity than that measured in a comparable yeast cell of the same species. The term reduced also refers to the elimination of polypeptide activity as compared to a comparable yeast cell of the same species. Thus, yeast cells lacking activity for an endogenous PDC, GPD, ALDH, or 3-KAR are considered to have reduced activity for PDC, GPD, ALDH, or 3-KAR since most, if not all, comparable yeast strains have at least some activity for PDC, GPD, ALDH, or 3-KAR. Such reduced PDC, GPD, ALDH, or 3-KAR activities can be the result of lower PDC, GPD, ALDH, or 3-KAR concentration (e.g., via reduced expression), lower specific activity of the PDC, GPD, ALDH, or 3-KAR, or a combination thereof. Many different methods can be used to make yeast having reduced PDC, GPD, ALDH, or 3-KAR activity. For example, a yeast cell can be engineered to have a disrupted PDC-, GPD-, ALDH-, or 3-KAR-encoding locus using common mutagenesis or knock-out technology. See, e.g., Methods in Yeast Genetics (1997 edition). Adams, Gottschling, Kaiser, and Stems, Cold Spring Harbor Press (1998). In addition, a yeast cell can be engineered to partially or completely remove the coding sequence for a particular PDC, GPD, ALDH, or 3-KAR. Furthermore, the promoter sequence and/or associated regulatory elements can be mutated, disrupted, or deleted to reduce the expression of a PDC, GPD, ALDH, or 3-KAR. Moreover, certain point-mutation(s) can be introduced which results in a PDC, GPD, ALDH, or 3-KAR with reduced activity. Also included within the scope of this invention are yeast strains which when found in nature, are substantially free of one or more PDC, GPD, ALDH, or 3-KAR activities.

Alternatively, antisense technology can be used to reduce PDC, GPD, ALDH, or 3-KAR activity. For example, yeasts can be engineered to contain a cDNA that encodes an antisense molecule that prevents a PDC, GPD, ALDH, or 3-KAR from being made. The term “antisense molecule” as used herein encompasses any nucleic acid molecule that contains sequences that correspond to the coding strand of an endogenous polypeptide. An antisense molecule also can have flanking sequences (e.g., regulatory sequences). Thus antisense molecules can be ribozymes or antisense oligonucleotides. A ribozyme can have any general structure including, without limitation, hairpin, hammerhead, or axhead structures, provided the molecule cleaves RNA.

### Overexpression of Heterologous Genes

Methods for overexpressing a polypeptide from a native or heterologous nucleic acid molecule are well known. Such methods include, without limitation, constructing a nucleic acid sequence such that a regulatory element promotes the expression of a nucleic acid sequence that encodes the desired polypeptide. Typically, regulatory elements are DNA sequences that regulate the expression of other DNA sequences at the level of transcription. Thus, regulatory elements include, without limitation, promoters, enhancers, and the like. For example, the exogenous genes can be under the control of an inducible promoter or a constitutive promoter. Moreover, methods for expressing a polypeptide from an exogenous nucleic acid molecule in yeast are well known. For example, nucleic acid constructs that are used for the expression of exogenous polypeptides within Kluyveromyces and Saccharomyces are well known (see, e.g., U.S. Pat. Nos. 4,859,596 and 4,943,529, for Kluyveromyces and, e.g., Gellissen et al., Gene 190(1):87-97 (1997) for Saccharomyces). Yeast plasmids have a selectable marker and an origin of replication. In addition certain plasmids may also contain a centromeric sequence. These centromeric plasmids are generally a single or low copy plasmid. Plasmids without a centromeric sequence and utilizing either a 2 micron (S. cerevisiae) or 1.6 micron (K. lactis) replication origin are high copy plasmids. The selectable marker can be either prototrophic, such as HIS3, TRP1, LEU2, URA3 or ADE2, or antibiotic resistance, such as, bar, ble, hph, or kan.

In another embodiment, heterologous control elements can be used to activate or repress expression of endogenous genes. Additionally, when expression is to be repressed or eliminated, the gene for the relevant enzyme, protein or RNA can be eliminated by known deletion techniques.

As described herein, any yeast within the scope of the disclosure can be identified by selection techniques specific to the particular polypeptide (e.g. an isobutanol pathway enzyme) being expressed, over-expressed or repressed. Methods of identifying the strains with the desired phenotype are well known to those skilled in the art. Such methods include, without limitation, PCR, RT-PCR, and nucleic acid hybridization techniques such as Northern and Southern analysis, altered growth capabilities on a particular substrate or in the presence of a particular substrate, a chemical compound, a selection agent and the like. In some cases, immunohistochemistry and biochemical techniques can be used to determine if a cell contains a particular nucleic acid by detecting the expression of the encoded polypeptide. For example, an antibody having specificity for an encoded enzyme can be used to determine whether or not a particular yeast cell contains that encoded enzyme. Further, biochemical techniques can be used to determine if a cell contains a particular nucleic acid molecule encoding an enzymatic polypeptide by detecting a product produced as a result of the expression of the enzymatic polypeptide. For example, transforming a cell with a vector encoding acetolactate synthase and detecting increased acetolactate concentrations compared to a cell without the vector indicates that the vector is both present and that the gene product is active. Methods for detecting specific enzymatic activities or the presence of particular products are well known to those skilled in the art. For example, the presence of acetolactate can be determined as described by Hugenholtz and Starrenburg, 1992, Appl. Micro. Biot. 38:17-22.

### Increase of Enzymatic Activity

Yeast microorganisms of the invention may be further engineered to have increased activity of enzymes (e.g., increased activity of enzymes involved in an isobutanol producing metabolic pathway). The term “increased” as used herein with respect to a particular enzymatic activity refers to a higher level of enzymatic activity than that measured in a comparable yeast cell of the same species. For example, overexpression of a specific enzyme can lead to an increased level of activity in the cells for that enzyme. Increased activities for enzymes involved in glycolysis or the isobutanol pathway would result in increased productivity and yield of isobutanol.

Methods to increase enzymatic activity are known to those skilled in the art. Such techniques may include increasing the expression of the enzyme by increased copy number and/or use of a strong promoter, introduction of mutations to relieve negative regulation of the enzyme, introduction of specific mutations to increase specific activity and/or decrease the K_{M} for the substrate, or by directed evolution. See, e.g., Methods in Molecular Biology (vol. 231), ed. Arnold and Georgiou, Humana Press (2003).

### Methods of Using Recombinant Microorganisms for Metabolite Production

For a biocatalyst to produce a beneficial metabolite most economically, it is desirable to produce said metabolite at a high yield. Preferably, the only product produced is the desired metabolite, as extra products (i.e. by-products) lead to a reduction in the yield of the desired metabolite and an increase in capital and operating costs, particularly if the extra products have little or no value. These extra products also require additional capital and operating costs to separate these products from the desired metabolite.

In one aspect, the present application provides methods of producing a desired metabolite using a recombinant described herein. In one embodiment, the recombinant microorganism comprises a KARI-requiring biosynthetic pathway, wherein said recombinant microorganism comprises at least one nucleic acid molecule encoding a KARI that is at least about 60% identical to SEQ ID NO: 2 and/or SEQ ID NO: 12. In one embodiment, the KARI is derived from the genus Slackia. In a specific embodiment, the KARI is derived from Slackia exigua. In another specific embodiment, the KARI is encoded by SEQ ID NO: 1. In another embodiment, the KARI is derived from the genus Cryptobacterium. In a specific embodiment, the KARI is derived from Cryptobacterium curtum. In another specific embodiment, the KARI is encoded by SEQ ID NO: 3. In yet another embodiment, the KARI is derived from the genus Eggerthella. In a specific embodiment, the KARI is derived from Eggerthella lenta. In another specific embodiment, the KARI is encoded by SEQ ID NO: 5, SEQ ID NO: 7, or SEQ ID NO: 9. In yet another embodiment, the KARI has one or more modifications or mutations at positions corresponding to amino acids selected from: (a) tyrosine 35 of the S. exigua KARI (SEQ ID NO: 2); (b) leucine 57 of the S. exigua KARI (SEQ ID NO: 2); (c) arginine 58 of the S. exigua KARI (SEQ ID NO: 2); (d) glycine 60 of the S. exigua KARI (SEQ ID NO: 2); (e) serine 61 of the S. exigua KARI (SEQ ID NO: 2); (f) serine 62 of the S. exigua KARI (SEQ ID NO: 2); (g) serine 63 of the S. exigua KARI (SEQ ID NO: 2); (h) isoleucine 95 of the S. exigua KARI (SEQ ID NO: 2); and (i) valine 99 of the S. exigua KARI (SEQ ID NO: 2).

In an exemplary embodiment, the KARI-requiring biosynthetic pathway is a pathway for the production of a metabolite selected from isobutanol, isoleucine, leucine, valine, pantothenate, coenzyme A, 1-butanol, 2-methyl-1-butanol, 3-methyl-1-butanol, 3-methyl-1-pentanol, 4-methyl-1-pentanol, 4-methyl-1-hexanol, and 5-methyl-1-heptanol. In a further exemplary embodiment, the beneficial metabolite is isobutanol.

In a method to produce a beneficial metabolite (e.g., isobutanol) from a carbon source, the recombinant microorganism is cultured in an appropriate culture medium containing a carbon source. In certain embodiments, the method further includes isolating the beneficial metabolite (e.g., isobutanol) from the culture medium. For example, a beneficial metabolite (e.g., isobutanol) may be isolated from the culture medium by any method known to those skilled in the art, such as distillation, pervaporation, or liquid-liquid extraction. In certain exemplary embodiments, the beneficial metabolite is selected from isobutanol, isoleucine, leucine, valine, pantothenate, coenzyme A, 1-butanol, 2-methyl-1-butanol, 3-methyl-1-butanol, 3-methyl-1-pentanol, 4-methyl-1-pentanol, 4-methyl-1-hexanol, and 5-methyl-1-heptanol. In a further exemplary embodiment, the beneficial metabolite is isobutanol.

In one embodiment, the recombinant microorganism may produce the beneficial metabolite (e.g., isobutanol) from a carbon source at a yield of at least 5 percent theoretical. In another embodiment, the microorganism may produce the beneficial metabolite (e.g., isobutanol) from a carbon source at a yield of at least about 10 percent, at least about 15 percent, about least about 20 percent, at least about 25 percent, at least about 30 percent, at least about 35 percent, at least about 40 percent, at least about 45 percent, at least about 50 percent, at least about 55 percent, at least about 60 percent, at least about 65 percent, at least about 70 percent, at least about 75 percent, at least about 80 percent, at least about 85 percent, at least about 90 percent, at least about 95 percent, or at least about 97.5% theoretical. In a specific embodiment, the beneficial metabolite is isobutanol.

### Distillers Dried Grains Comprising Spent Yeast Biocatalysts

In an economic fermentation process, as many of the products of the fermentation as possible, including the co-products that contain biocatalyst cell material, should have value. Insoluble material produced during fermentations using grain feedstocks, like corn, is frequently sold as protein and vitamin rich animal feed called distillers dried grains (DDG). See, e.g., commonly owned and co-pending U.S. Publication No. 2009/0215137, which is herein incorporated by reference in its entirety for all purposes. As used herein, the term “DDG” generally refers to the solids remaining after a fermentation, usually consisting of unconsumed feedstock solids, remaining nutrients, protein, fiber, and oil, as well as spent yeast biocatalysts or cell debris therefrom that are recovered by further processing from the fermentation, usually by a solids separation step such as centrifugation.

Distillers dried grains may also include soluble residual material from the fermentation, or syrup, and are then referred to as “distillers dried grains and solubles” (DDGS). Use of DDG or DDGS as animal feed is an economical use of the spent biocatalyst following an industrial scale fermentation process.

Accordingly, in one aspect, the present invention provides an animal feed product comprised of DDG derived from a fermentation process for the production of a beneficial metabolite (e.g., isobutanol), wherein said DDG comprise a spent yeast biocatalyst of the present invention. In an exemplary embodiment, said spent yeast biocatalyst has been engineered to comprise at least one nucleic acid molecule encoding a KARI that is at least about 60% identical to SEQ ID NO: 2 and/or SEQ ID NO: 12. In one embodiment, the KARI is derived from the genus Slackia. In a specific embodiment, the KARI is derived from Slackia exigua. In another specific embodiment, the KARI is encoded by SEQ ID NO: 1. In another embodiment, the KARI is derived from the genus Cryptobacterium. In a specific embodiment, the KARI is derived from Cryptobacterium curtum. In another specific embodiment, the KARI is encoded by SEQ ID NO: 3. In yet another embodiment, the KARI is derived from the genus Eggerthella. In a specific embodiment, the KARI is derived from Eggerthella lenta. In another specific embodiment, the KARI is encoded by SEQ ID NO: 5, SEQ ID NO: 7, or SEQ ID NO: 9. In yet another embodiment, the KARI has one or more modifications or mutations at positions corresponding to amino acids selected from: (a) tyrosine 35 of the S. exigua KARI (SEQ ID NO: 2); (b) leucine 57 of the S. exigua KARI (SEQ ID NO: 2); (c) arginine 58 of the S. exigua KARI (SEQ ID NO: 2); (d) glycine 60 of the S. exigua KARI (SEQ ID NO: 2): (e) serine 61 of the S. exigua KARI (SEQ ID NO: 2); (f) serine 62 of the S. exigua KARI (SEQ ID NO: 2); (g) serine 63 of the S. exigua KARI (SEQ ID NO: 2); (h) isoleucine 95 of the S. exigua KARI (SEQ ID NO: 2); and (i) valine 99 of the S. exigua KARI (SEQ ID NO: 2).

In certain additional embodiments, the DDG comprising a spent yeast biocatalyst of the present invention comprise at least one additional product selected from the group consisting of unconsumed feedstock solids, nutrients, proteins, fibers, and oils.

In another aspect, the present invention provides a method for producing DDG derived from a fermentation process using a yeast biocatalyst (e.g., a recombinant yeast microorganism of the present invention), said method comprising: (a) cultivating said yeast biocatalyst in a fermentation medium comprising at least one carbon source; (b) harvesting insoluble material derived from the fermentation process, said insoluble material comprising said yeast biocatalyst; and (c) drying said insoluble material comprising said yeast biocatalyst to produce the DDG.

In certain additional embodiments, the method further comprises step (d) of adding soluble residual material from the fermentation process to said DDG to produce DDGS. In some embodiments, said DDGS comprise at least one additional product selected from the group consisting of unconsumed feedstock solids, nutrients, proteins, fibers, and oils.

This invention is further illustrated by the following examples that should not be construed as limiting. The contents of all references, patents, and published patent applications cited throughout this application, as well as the Figures and the Sequence Listing, are incorporated herein by reference for all purposes.

### Example 1

### Reduction of KARI Inhibition by 2,3-dihydroxyisovalerate (DHIV)

The purpose of this example is to show that the inhibition of KARI by its product DHIV limits the activity of this enzyme in isobutanol production strains.

### Materials and Methods for Example 1

TABLE 2
Strains and Plasmids Used in Example 1.

| GEVO No. | Genotype/Source |
|---|---|
| GEVO3121 | MATa ura3 leu2 his3 trp1 pdc1::P_{CUP1-1}-Bs_alsS_coSc, TRP1, pGV2398 |
| GEVO2962 | MATa ura3 leu2 his3 trp1 pdc1::P_{CUP1}:Bs_alsS_coSc:T_{CYC1}:P_{PGK1}:Ll_kivD:P_{ENO2}:Sp_HIS5 |
| | pdc5::LEU2:bla:P_{TEF1}:Sc_ILV3ΔN:P_{TDH3}:Ec_ilvC_coSc_Q110V |
| | pdc:6::URA3:bla:P_{TEF1}:Ll_kivD:P_{TDH3}:DmADH |
| pGV2227 | PSc_TEF1:Ll_ilvD_coSc, PSc_TDH3:Ec_ilvC_coScQ110V, PSc_TPI1:G418R, PSc_PGK1:Ll_kivD, |
| | PSc_ENO2:Ll_adhA, 2μ origin of replication, ApR, pMB1 origin of replication |
| pGV2398 | PSc_TEF1:Ll_ilvD_coSc, PSc_TDH3:Ec_ilvC_coScQ110V_his6, PSc_TPI1:G418R, PSc_PGK1: |
| | Ll_kivD, PSc_ENO2:Ll_adhA, 2μ origin of replication, ApR, pMB1 origin of replication |

Fermentation:

Two-stage growth and production fermentations of Saccharomyces cerevisiae, strains GEVO2962 (PDC-minus production strain) and GEVO3121 (PDC-plus production strain) were performed. The strains were growth to a target OD600 of 10 at high aeration, then switched to low OTR conditions for production. Samples were taken at the switch to production, at mid-production and at the end of the fermentations. Sampling times (EFT) were: 40, 48, 70.3 h. Broth samples were analyzed for products using GC and HPLC. Analysis for DHIV was done using LC4. At the mid-production timepoint (48 h) samples for intracellular metabolite analysis were taken in addition.

Fast Sampling for Intracellular Metabolite Analysis:

Samples were taken using a custom-made set up. Each sampling device consisted of a hard piped fermenter dip tube, plug valve, normally closed electromagnetic solenoid actuated pinch valve, relay timer, 24 volt power supply, sample container dip tube and a deep vacuum source (<50 mbar) which in this case was a stand alone pump. The fermenter dip tube and the sample container dip tube were connected with a short piece of size 15 silicone tubing (˜5 cm) to allow the placement of the solenoid actuated pinch valve which controlled the sample time. Hard pipe was used where possible to minimize inconsistencies due to flexible tubing collapse. A 24 volt system was selected as a safety feature. The sample assembly was autoclaved with the fermenter to maintain sterile operation.

To prepare for sample collection, a collection vessel (250 mL Schott flask) with a pre-measured aliquot of quench solution (150 mL methanol) that was pre-cooled to −40° C. was attached to the dip tube. The vacuum pump was turned on and a vacuum was pulled on the collection vessel up to the pinch valve. Once the desired vacuum pressure was attained (30 mbar), the plug valve was opened to create a path from the fermenter to the collection vessel and the timer actuated. The timer energized the solenoid pinch valve which opened it for the timer set time (1 s). After the elapsed time, the relay timer immediately de-energized the solenoid and the pinch valve closed by internal spring action.

Extraction of Intracellular Metabolites:

For each fermenter at mid production two samples were taken using the fast sampling method described above. 2×25 mL were sampled into Schott flask with 150 mL MeOH (−44° C.); the flasks were weighed before and after sampling to measure the sample volume. From the time of sampling the samples were kept as close to −40° C. as possible. The samples were kept in a cryostat (Lauda) adjusted to −40° C. The samples were divided into 4×50 mL Falcon tubes (40 mL each). The tubes were centrifuged at 5000 g for 5 min in a centrifuge rotor that was cooled to −40° C. in a centrifuge cooled to −11° C. The supernatant was decanted and 40 mL methanol (−40° C.) were added to each tube. The samples were centrifuged at 5000 g for 5 min in a second centrifuge rotor that was cooled to −40° C. in a centrifuge cooled to −11° C. The supernatant was decanted and the pellets were stored in a −40° C. freezer until extraction.

To one cell pellet a mix of standards was added before extraction and to a second pellet the same standard mix was added after extraction and evaporation. 15 mL of boiling ethanol was added to the Falcon tubes. The tubes were vortexed for 30 s and incubated in the 90° C. bath for 5 min. The samples were transferred to the cryostat, then centrifuged at 5000 g for 5 min at −11° C. The supernatant was transferred to 100 mL anaerobic flasks and the volume of the extracts was reduced to below 1 mL using a speed vac (Labconco). The extracts were transferred to microcentrifuge tubes and the volume was adjusted to 1 mL with water. The extracts were centrifuged and the supernatants were filtered through 0.2 μm syringe filters before submission to Analytics.

High Performance Liquid Chromatography LC4:

Analysis for DHIV was performed on a HP-1100 High Performance Liquid Chromatography system equipped with an IonPac AS11-HC Analytical, IonPac AG11-HC guard column (3-4 mm for IonPac ATC column) or equivalent and an IonPac ATC-1 Anion Trap column or equivalent. Oxo acids were detected using a conductivity detector (ED50-suppressed conductivity, Suppressor type: ASRS 4 mm in AutoSuppression recycle mode. Suppressor current: 300 mA). The column temperature was 35° C. This method used the following elution profile: 0.25 mM NaOH for 10 min, linear gradient to 3.5 mM NaOH at 25 min, linear gradient to 38.5 mM at 37 min, linear gradient to 0.25 mM at 37.1 min. Flow was set at 2 mL/min. Injection size is 5 μL.

KARI Enzyme Assay:

Ketol-acid reductoisomerase (KARI) activity was determined as follows: The following stock solutions were prepared: 1 M potassium phosphate, pH 7, 1 M MgCl₂, 100 mM DTT, 25 mM NADPH, 200 mM acetolactate. The acetolactate was made fresh each time by mixing 50 μL Ethyl-2-acetoxy-2-methylacetoacetate (EAMAA) with 990 μL water. Gradually 260 μL of 2 N NaOH was added in 10 μL increments to the EAMAA water mixture. After each addition the sample was vortexed for 15 seconds, and the procedure repeated until the entire 260 μL of 2 N NaOH was added. Afterwards the solution was mixed on an orbital shaker for 20 minutes (Krampitz, 1957). KARI reaction buffer (100 μL per reaction) was prepared to the following concentrations: 250 mM potassium phosphate, pH 7, 10 mM MgCl₂, 1 mM DTT, 0.2 mM NADPH, 10 mM acetolactate. For the no substrate controls, the acetolactate was substituted with water. 10 μL of each lysate were transferred into a 96 well half area assay plate (Greiner Bio-one product #675801). The reaction was started with 90 μL of reaction buffer to each well using a multichannel pipette. The samples were mixed immediately. The samples were read at 340 nm every 10 s for 5 min.

Results:

Some KARI enzymes have previously been shown to be inhibited by DHIV as shown for the Salmonella typhimurium KARI (Chunduru et al., Biochemistry, Vol. 28, 1989, 486-493), and the Spinacea oleracea KARI (Dumas et al., Biochemistry Journal, Vol. 288, 1992, 865-874). The inhibition constant for E. coli IIvC was measured at Kᵢ=0.1 mM (Example 5, Table 11). Analysis of DHIV concentrations in samples taken from fermentations of isobutanol production strains at 48 hrs show that GEVO2962 produced 0.65+0.06 g/L and 0.25±0.15 g/L of DHIV in broth and intracellularly, respectively, while GEVO3121 produced 2.1±0.03 g/L and 0.28±0.21 g/L of DHIV in broth and intracellularly, respectively. At these concentrations, the KARI of isobutanol-producing strains would be inhibited. Thus, the limited isobutanol productivity of currently-existing strains can be attributed in part to inhibition of the pathway enzyme KARI by DHIV.

### Example 2

The purpose of this example is to show that an NKR derived from the E. coli KARI is inhibited by DHIV and that this inhibition affects NKR activity in isobutanol production strains.

### Materials and Methods for Example 2

Medium Components:

The medium included 20 g/L of Difco Yeast Extract, 30 g/L of Difco Peptone, 50 g/L of Glucose, 5 g/L of ethanol and 0.5 g/L of MgSO₄.

Fermentation Conditions:

Two 80 mL shake flask cultures were inoculated with one 2 mL glycerol stock. The cultures were grown in 500 mL baffled shake flasks at 30° C., 250 rpm for 20 h. Cultures were diluted 3× and further incubated to a final OD of 12-13. The fermenters were inoculated at OD 0.3-0.4 with GEVO6143 in B6 and B8. The strain was grown at high aeration (10% dissolved oxygen (DO)) until agitation maxed out at 700 rpm, then DO was allowed to drop and at 20.5 h the fermenters were switched to low OTR conditions (0.3-0.4 mM/h) for production. Samples for intracellular metabolite (IM) analysis were taken at mid production and at the final timepoint.

Fast Sampling for Intracellular Metabolite Analysis:

Samples were taken using a custom-made set up: The sampling device consisted of a hard piped fermenter dip tube, plug valve, normally closed electromagnetic solenoid actuated pinch valve, relay timer, 24 volt power supply, sample container dip tube and a deep vacuum source (<50 mbar) which in this case was a standalone pump. The fermenter dip tube and the sample container dip tube were connected with a short piece of size 15 silicone tubing (˜5 cm) to allow the placement of the solenoid actuated pinch valve which controlled the sample time. Hard pipe was used where possible to minimize inconsistencies due to flexible tubing collapse. A 24 volt system was selected as a safety feature.

To prepare for sample collection, a collection vessel which is a 250 mL vacuum rated Schott flask (Schott North America, Inc., Elmsford, N.Y., USA. Fisher cat#NC9690963) with a pre-measured aliquot of quench solution (110 mL methanol) that was pre-cooled to −40° C. was attached to the dip tube. The vacuum pump was turned on and a vacuum was pulled on the collection vessel up to the pinch valve. Once the desired vacuum pressure was attained (30 mbar), the plug valve was opened to create a path from the fermenter to the collection vessel and the timer actuated. The timer energized the solenoid pinch valve which opened it for the timer set time (1 s). After the elapsed time, the relay timer immediately de-energized the solenoid and the pinch valve closed by internal spring action. To collect additional samples from the same fermenter, a blank collection vessel was installed and air was allowed to back flow through the plug valve. The back flow was accomplished by use of a syringe connected to the collection vessel through a sterile filter. Once the tubing was clear of broth as indicated by bubbling from the fermenter dip tube, the plug valve was closed thus insuring a consistent starting point relative to tubing hold up volume for the next sample. The next sample was collected by repeating the above process.

Samples were taken from fermenter B6 at two different time points (33.5 h, and 62.5 h) using the fast sampling method described above. Approximately 25 mL were sampled into collection vessels with 110 mL MeOH (−40° C.); the flasks were weighed before and after sampling to measure the sample volume. From the time of sampling the samples were kept as close to −40° C. as possible. The samples were kept in a cryostat (Proline RP1845, Lauda, Germany) adjusted to −40° C. Each sample was divided into 3×40 mL custom-made conical bottom glass tubes. The tubes were centrifuged at 3,000 g for 5 minutes in a centrifuge rotor that was cooled to −40° C. in a centrifuge cooled to −11° C. The supernatant was decanted and cell pellets were washed with 30 mL methanol (−40° C.). The cell pellets were resuspended by vortexing on a VXR basic Vibrax platform vortexer (IKA, Wilmington, N.C., USA) in a −40° C. freezer. The samples were centrifuged at 3,000 g for 5 min in a second centrifuge rotor that was cooled to −40° C. in a centrifuge cooled to −11° C. The supernatants were decanted and the pellets were stored at −40° C. until extraction. The decanted quench and wash solutions were collected and stored at −40° C. until analysis.

Cell Extraction for Intracellular Metabolite Analysis:

Methanol-chloroform cell extractions for intracellular metabolite analysis was based on Canelas, A. B., et al. (2009) Analytical Chemistry 81(17): 7379-7389. During the extraction process, samples were kept as close to −40° C. as possible by performing the work in a cryostat set to −40° C. 16 mL of 50% MeOH pre-cooled to −40° C. were added to each cell pellet (in glass tubes). The pellets were resuspended in 200 μL of 20 mM Tris-HCl, pH 8.5 by vortexing on the platform vortexer (IKA. Wilmington, N.C., USA) in a −40° C. freezer. 16 mL of CHCl₃ pre-cooled to −40° C. were then added to each cell suspension. The samples were vortexed at −40° C. for 45 min, and then centrifuged at 3.000 g for 5 min in a centrifuge rotor that was cooled to −40° C. in a centrifuge cooled to −11° C. The centrifuge rotor was returned to the −40° C. freezer while the following work was performed. The upper, aqueous 50% MeOH phases of the extractions were removed with a 5 mL pipette tip and transferred to a 50 mL Falcon tube pre-cooled to −40° C. in the cryostat. The lower organic CHCl₃ phases were re-extracted with 16 mL 50% MeOH (−40° C.). After addition of the MeOH, the samples were vortexed on the platform vortexer in the −40° C. freezer for 30 s. The samples were then centrifuged at 3,000 g for 5 min in the same rotor as before that was cooled to −40° C. in a centrifuge cooled to −11° C. The upper aqueous phases were removed as described above and pooled with the previous extracts. To concentrate the extracts, samples were transferred to 100 mL RapidVap tubes for use with the RapidVap evaporation system (Labconco, Kansas City, Mo. USA). In order to concentrate the extracts to a volume of ≦2.0 mL, the samples were incubated in the RapidVap set to 40° C. with shaking at full speed for approximately 2 hours. The extracts were transferred to microcentrifuge tubes and the volume of each extract was adjusted to 2 mL with water. The extracts were centrifuged at 21,500 g for 10 min at 4° C. and the supernatants were filtered through 0.2 μm syringe filters before submission to Analytics for method LC4. Recovery and matrix effects were quantitated using internal standards.

High Performance Liquid Chromatography LC4:

Analysis of dihydroxyisovalerate was performed on a HP-1100 High Performance Liquid Chromatography system equipped with an IonPac AS11-HC Analytical (Dionex: 9 μm, 4.6×250 mm) coupled with an IonPac AG11-HC guard column (Dionex: 13 μm, 4.6×50 mm) and an IonPac ATC-1 Anion Trap column (Dionex: 9×24 mm). 2-acetolactate is detected using a UV detector at 225 nm, while all other analytes were detected using a conductivity detector (ED50-suppressed conductivity, Suppressor type: ASRS 4 mm in AutoSuppression recycle mode, Suppressor current: 300 mA). The column temperature was 35° C. This method used the following elution profile: 0.25 mM NaOH for 3 min, linear gradient to 5 mM NaOH at 25 min, linear gradient to 38.25 mM at 25.1 min, hold at 38.25 mM until 30 min, linear gradient to 0.25 mM at 30.1 min, re-equilibrate at 0.25 mM for 7 min. Flow was set at 2 mL/min. Injection size is 10 μL. Analysis was performed using standards prepared from DHIV (2,3-dihidroxy-3-methyl-butanoate, CAS 1756-18-9, which was custom synthesized at Caltech (Cioffi et al., Anal Biochem 104 pp. 485 (1980)).

NKR Assay in Microtiter Plates with Purpose to Measure Inhibition:

NKR activity was assayed kinetically by monitoring the decrease in NADH concentration by measuring the absorbance at 340 nm. A reaction buffer was prepared containing 250 mM potassium phosphate pH 7, 1 mM DTT, 200 μM NADH, 2.5 mM (S)-2-acetolactate, and 10 mM MgCl₂. Ten μL purified enzyme were placed into a microtiter plate. The reaction was initiated by addition of 90 μL of the reaction buffer. The kinetics with respect to substrate conversion in presence of the inhibitor were determined by varying the substrate concentration (5.1, 2.55, 1.275, 0.638, 0.319, 0.159, and 0.0799 mM), while keeping the cofactor concentration constant at 200 μM. The inhibition constant was measured by repeating these measurements with different amounts of inhibitor (0, 0.07, 0.13, 0.26, 0.51, and 1.07 mM, final concentrations).

Results:

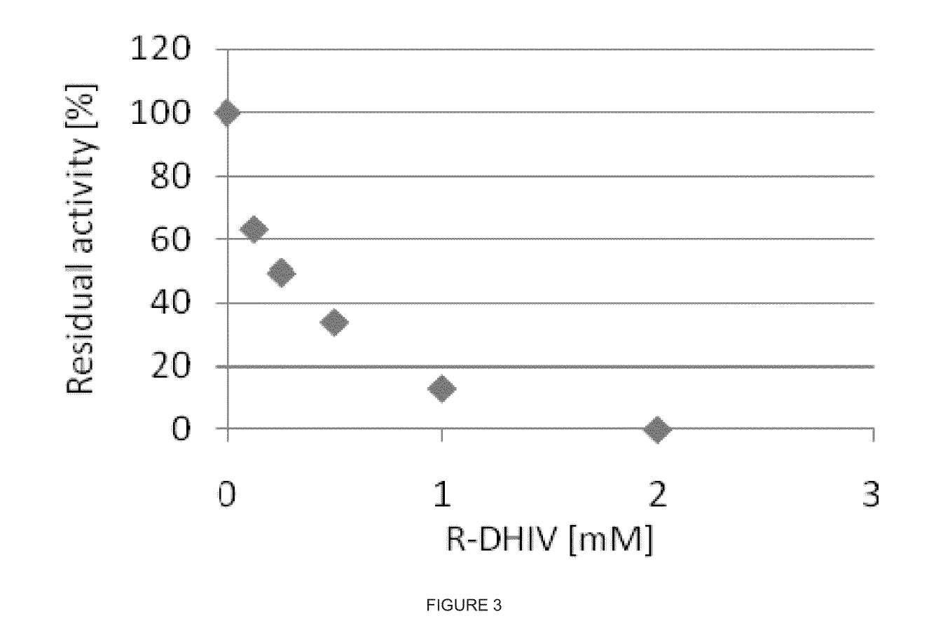

The intracellular levels of DHIV in GEVO6143, an S. cerevisiae strain comprising the E. coli KARI, were 1.5 (+/−0.2) mM and 1.8 (+/−0.14) mM at 62.5 h. As shown in FIG. 3, the NKR used in GEVO6143 (Ec_ilvC^{P2D1-A1}, described in US 2010/0143997) is inhibited by its product DHIV in vitro. The K of Ec_ilvC^{P2D1-A1} for DHIV was 0.045 (+/−0.011) mM.

### Example 3

### Identification of a High-Performance KARI from Slackia exigua

The purpose of this example is to show how a high-performance KARI from Slackia exigua was identified.

### Materials and Methods for Example 3

TABLE 4
Strain Used in Example 3.

| | |
|---|---|
| GEVO3956 | MATa ura3 leu2 his3 trp1 ald6::P_{ENO2}-Ll_adhA^{RE1}-P_{FBA1}-Sc_TRP1 gpd1::T_{KI}_URA3 |
| | gpd2::T_{KI}_URA3 tma29::T_{KI}_URA3 pdc1::P_{PDC1}-Ll_kivD2_coSc5-P_{FBA1}-LEU2-T_{LEU2}-P_{ADH1}- |
| | Bs_alsS1_coSc-T_{CYC1}-P_{PGK1}-Ll_kivD2_coEc-P_{ENO2}-Sp_HIS5 pdc5::T_{KI}_URA3 |
| | pdc6::P_{TDH3}-Sc_AFT1-P_{ENO2}- Ll_adhA^{RE1}-T-_{KI}_URA3_short-P_{FBA1}-KI_URA3-T_{KI}_URA3 |
| | {evolved for C2 supplement-independence, glucose tolerance and faster growth} |

TABLE 5
Plasmids Used in Example 3.

| | |
|---|---|
| pGV3009 | P_{Sc}_TEF1:Ll_ilvD_coSc:T_{Sc}_ADH1, P_{Sc}_PDC1-350:Ec_ilvC_coSc^{P2D1}_A1_his6, P_{Sc}_TPI1:G418^{R}, |
| | P_{Sc}_ENO2:Ll_adhA^{RE1}, CEN/ARS origin of replication, Ap^{R}, pMB1 origin of replication |
| pGV3022 | P_{Sc}_TEF1:Ll_ilvD_coSc:T_{Sc}_ADH1, P_{Sc}_PDC1-350:Ec_ilvC_coSc^{his6}, P_{Sc}_TPI1:G418^{R}, |
| | P_{Sc}_ENO2:Ll_adhA^{RE1}, CEN/ARS origin of replication, Ap^{R}, pMB1 origin of replication |
| pGV3012 | P_{Sc}_TEF1:Ll_ilvD_coSc:T_{Sc}_ADH1, P_{Sc}_TPI1:G418^{R}, P_{Sc}_ENO2:Ll_adhA^{RE1}, CEN/ARS origin of |
| | replication, Ap^{R}, pMB1 origin of replication |

In this example, a series of KARI genes were individually expressed from a yeast promoter in conjunction with other components of an isobutanol production pathway in yeast such that KARI was the limiting enzyme in the pathway and the amount of isobutanol produced during a fermentation was dependent on the KARI activity level. In this system, the S. cerevisiae host strain GEVO3956 was used to produce isobutanol when supplied with an isobutanol production pathway plasmid expressing ALS and KIVD, and a low copy number plasmid expressing KARI, DHAD, and ADH enzymes.

KARIs were identified and grouped by bioinformatic and phylogenetic methods based on the amino acid sequence. Individual KARIs were chosen for the above analysis to provide a representative sample of broadly diverse clades. KARI genes were designed and synthesized based on the primary amino acid sequence of the chosen KARI, with codon optimization of the genes for expression in S. cerevisiae.

Shake Flask Fermentations:

Shake flask fermentations using GEVO3956 carrying these individual plasmids were performed together with GEVO3956 carrying pGV3022 (derived from pGV3009 but containing the E. coli ilvC-coSc gene expressed from the Sc_PDC1⁻³⁵⁰ promoter) and GEVO3956 carrying pGV3012 (equivalent to pGV3009 lacking the Sc_PDC1⁻³⁵⁰ promoter and KARI gene) for comparison of isobutanol production. The shake flask fermentations were performed as follows. The strains were grown overnight in 3 mL of YPD medium containing 1% v/v ethanol and 0.1 g/L G418 at 30° C. and 250 rpm. The OD₆₀₀ of these cultures was determined after overnight growth and the appropriate amount of culture was added to 50 mL of YP medium containing 5% w/v glucose, 1% v/v ethanol. 200 mM MES, pH 6.5, and 0.1 g/L G418 to obtain an OD₆₀₀ of 0.1 in 250 mL baffled flasks with sleeve caps. Cultures were incubated at 30° C. and 250 rpm overnight. The OD₆₀₀ of these cultures was determined after overnight growth and the appropriate amount of culture to total 250 ODs was added to 50 mL Falcon tubes and centrifuged at 2700×g for 5 minutes. The supernatant was removed and cells were resuspended in 50 mL of YP medium containing 8% w/v glucose, 1% v/v ethanol, 200 mM MES, pH 6.5, and 0.1 g/L G418 to obtain a final OD₆₀₀ of 5 OD per ml. At t=0 the OD₆₀₀ of each flask was determined. The fermentation cultures were incubated at 30° C. and 250 rpm in non-baffled 250 mL flasks with vented screw cap tops. After 24, 48 and 72 hours of incubation, 1.5 mL of culture was removed into 1.5 mL microcentrifuge tubes from each culture. OD₆₀₀ values were determined from the samples and the remainder of each sample was centrifuged for 10 min at 14,000 rpm in a microcentrifuge and 1 mL of the supernatant was removed to be submitted for gas chromatographic analysis. Analysis of volatile organic compounds, including ethanol and isobutanol, was performed on an Agilent 6890 gas chromatograph (GC) fitted with a 7683B liquid autosampler, a split/splitless injector port, a ZB-FFAP column (Phenomenex 30 m length, 0.32 mm ID, 0.25 μM film thickness) connected to a flame ionization detector (FID). The temperature program is as follows: 230° C. for the injector, 300° C. for the detector, 100° C. oven for 1 minute, 35° C./minute gradient to 230° C., and then hold for 2.5 min. Analysis is performed using authentic standards (>98%, obtained from Sigma-Aldrich), and a 6-point calibration curve with 1-pentanol as the internal standard. Injection size is 0.5 μL with a 50:1 split and run time is 7.4 min.

Results:

KARI gene clones resulting in isobutanol production equivalent to or higher than fermentations with pGV3022 or within two standard deviations below that of fermentations with pGV3022, averaged from multiple experiments, were chosen as encoding high-performing KARIs. These fermentations identified the Slackia exigua KARI from Slackia exigua strain ATCC 700122 (SEQ ID NO: 2) as a high-performance KARI. Table 6 shows the results of 48 hr and 72 hr isobutanol fermentation timepoints.

TABLE 6
Isobutanol Titers from Fermentations with the S. exigua and E. coli KARIs.

| Gene Expressed | 48 h isobutanol Titer (g/L) | 72 h isobutanol Titer (g/L) |
|---|---|---|
| Se1_KARI_coSc | 2.82 ± 0.11 | 4.40 ± 0.34 |
| (Mean of 3 experiments ± 1 standard | | |
| deviation | | |
| Ec_ilvC_coSc | 3.93 ± 1.16 | 4.72 ± 0.90 |
| (Mean of 3 experiments ± 2 standard | | |
| deviations) | | |

BLAST (BLASTP 2.2.25+(Altschul et al., 1990, J. Mol. Biol. 215: 403-10; Altschul et al., 2005, FEBS J. 272: 51019) and phylogenetic analysis revealed that the S. exigua KARI enzyme (SEQ ID NO: 2) shows significant identity to KARI enzymes from Cryptobacterium curtum (SEQ ID NO: 4) (71% identity), Eggerthella spp. (SEQ ID NO: 6) (68% identity), Eggerthella lenta (SEQ ID NO: 8) (68% identity), and Eggerthella spp. (SEQ ID NO: 10) (67% identity). S. exigua. C. curtum, and the Eggerthella strains all belong to the family Coriobacteriaceae of high GC Gram-positive bacteria in the phylum Actinobacteria, class Actinobacteria.

The alignments of the S. exigua KARI with the closely related KARIs from C. curtum and the Eggerthella strains indicates that the N-terminal 9 amino acids of the S. exigua KARI are not conserved and are missing from these related proteins. Additional analysis of the S. exigua KARI gene sequence annotated from the Slackia exigua genome sequence project (GenBank Acc. No. ACUX02000006) indicates that the annotated start of the S. exigua KARI protein is at the GTG codon with suboptimal ribosome binding sites (either due to spacing or sequence) while downstream and in-frame of the annotated GTG start codon is an ATG codon at nucleotide positions 374413-374415 with a predicted excellent ribosome binding site (both sequence and spacing, nucleotides 374391-374421 of the contig sequence from GenBank accession number ACUX02000006). This indicates that the actual start codon of S. exigua KARI gene is the ATG codon at nucleotide positions 374413-374415 and that a version of the S. exigua KARI lacking the N-terminal 9 amino acids would function as well or better for isobutanol production in yeast as compared with performance of S. exigua KARI of SEQ ID NO: 2. Such a protein would have the sequence of SEQ ID NO: 12.

### Example 4

### The S. exigua KARI Exhibits High Native NADH-Dependent Activity

As described in commonly owned and co-pending US Patent Publication No. 2010/0143997, NADH-dependent ketol-acid reductoisomerases (NKRs) are desirable in the context of biosynthetic pathways for the production of useful fuels or chemicals, including isobutanol. However, NKR enzymes have not been identified in nature. This example shows that the S. exigua KARI is an enzyme with high activity with the cofactor NADH, making it useful in applications that require an NKR and making it a valuable starting point for cofactor switch engineering with the aim of reducing or eliminating the activity of the enzyme with NADPH as a cofactor.

### Materials and Methods for Example 4

In this example, different KARI enzymes (Table 7) were screened for their activity with NADH and NADPH as cofactor. The enzymes were expressed in an S. cerevisiae strain by means of 2 micron plasmids carrying the KARI genes under the control of the S. cerevisiae TDH3 promoter. The transformants were cultivated in shake flask fermentations as described below and samples were taken after 44 h, centrifuged at 4000×g for 10 mins at 4° C. and the cell pellets were stored at −80° C. until analysis. The pellets were lysed by bead beating and the lysates were analyzed by KARI activity assay as described below using NADH and NADPH as cofactors.

TABLE 7
Plasmids Used in Example 4.

| Plasmid | KARI homolog source | Genotype |
|---|---|---|
| pGV3235 | Gramella forsetii | P_{SCTDH3}: Gf_KARI_coSc^{his6}, |
| | | P_{ScENO2}: Ll_adhA^{RE1} 2μ-ori, |
| | | pUC ori, bla, G418r |
| pGV3236 | Slackia exigua | P_{SCTDH3}: Se1_KARI_coSc^{his6}, |
| | | P_{ScENO2}: Ll_adhA^{RE1} 2μ-ori, |
| | | pUC ori bla, G418r |
| pGV3237 | Zymomonas mobilis | P_{SCTDH3}: Zm_KARI_coSc^{his6}, |
| | | P_{ScENO2}: Ll_adhA^{RE1} 2μ-ori, |
| | | pUC ori, bla, G418r |
| pGV3238 | Schizosaccharomyces | P_{SCTDH3}: Sp_ILV5_tr^{his6}, |
| | pombe* | P_{ScENO2}: Ll_adhA^{RE1} 2μ-ori, |
| | | pUC ori, bla, G418r |
| pGV3240 | Escherichia coli | P_{SCTDH3}: Ec_ilvC_coSc^{his6}, |
| | | P_{ScENO2}: Ll_adhA^{RE1} 2μ-ori, |
| | | pUC ori, bla, G418r |
| pGV3241 | none | P_{ScENO2}: Ll_adhA^{RE1} 2μ-ori, |
| | | pUC ori, bia, G418r |

*Truncated at the N-terminus to remove the mitochondrial targeting sequence.

Shake Flask Fermentations:

Patched cells (or single colonies) grown on YPD+G418 plates at 30° C. were mixed into 3 mL YP+12% dextrose+200 mM MES, pH 6.5+G418, in 14 mL culture tubes and incubated at 250 RPM, 30° C. for ˜24 hours. The cells from the cultures were used to inoculate YP+12% dextrose+200 mM MES, pH 6.5+G418, in non-baffled flasks with a vented grey screw-caps at OD₆₀₀ 0.01. The cultures were incubated at 250 RPM, 30° C. for 24 hours, diluted 1:3 and incubated for another 20 h at 250 RPM, 30° C. Samples for enzyme assay pellets and protein quantification pellets were taken from each shake flask, and cell-free supernatant was used to measure glucose levels in shake flasks.

KARI Enzyme Assay:

The assay and no substrate control reactions were performed in triplicate for each sample. The following stock solutions were prepared: 1 M potassium phosphate, pH 7, 0.1 M MgCl₂, 100 mM DTT, 25 mM NADH or NADPH, 200 mM 2-acetolactate. The 2-acetolactate was made fresh each time by mixing 50 μL Ethyl-2-acetoxy-2-methylacetoacetate (EAMAA) with 990 μL water. Gradually 260 μL of 2 N NaOH was added in 10 μL increments to the EAMAA water mixture. After each addition the sample was vortexed for 15 seconds, and the procedure repeated until the entire 260 μL of 2 N NaOH was added. Afterwards the solution was mixed on an orbital shaker for 20 min (Krampitz, 1957). KARI reaction buffer (100 μL per reaction) was prepared to the following concentrations: 250 mM potassium phosphate, pH 7, 10 mM MgCl₂, 1 mM DTT, 0.2 mM NADH or NADPH, 10 mM 2-acetolactate. For the no substrate controls, the 2-acetolactate was substituted with water. 10 μL of each lysate were transferred into a 96 well half area assay plate. The reaction was started with 90 μL of reaction buffer to each well using a multichannel pipette. The samples were mixed immediately. The samples were read at 340 nm every 10 s for 5 min.

Results:

The results of activity assays using KARI enzymes from S. exigua, E. coli, Z. mobilis, S. pombe, and G. forsetii are shown in Table 8.

TABLE 8
Specific activities measured on cell lysates prepared from
cell pellets taken from shake flask fermentations
of an S. cerevisiae strain expressing different KARI
homologs. The activities measured for the empty
vector controls were subtracted from the sample activities.

| | NADH-Dependent | Specific Activity |
|---|---|---|
| | Specific Activity | with NADH/Specific |
| KARI | [U/mg lysate] | Activity with NADPH |
| Se1 | 0.059 | 0.96 |
| Ec | 0.04 | 0.175 |
| Zm | 0.002 | 0.074 |
| Sp | <0.002* | <0.101 |
| Gf | <0.002* | <0.067 |

*The NADH dependent activities of these lysates were below the detection limit of the assay.
Se1: Slackia exigua,
Ec: Escherichia coli,
Zm: Zymomonas mobilis,
Sp: Schizosaccharomyces pombe,
Gf: Gramella forsetii

As Table 8 demonstrates, the S. exigua KARI showed similar activity with NADH and with NADPH as cofactor. Other KARI enzymes tested had below 20% activity with NADH as compared to the activity with NADPH.

Based upon these results, the S. exigua KARI shows several advantages over prior art KARIs. First, the engineering of a cofactor switched enzyme is time-consuming. An important factor dictating the speed and effectiveness of a cofactor switch is the availability of sufficient starting activity with the target cofactor. The S. exigua KARI is an enzyme with high starting activity reducing the time necessary for the engineering of a NKR enzyme. Second, the cofactor specificity of KARI has a significant physiological impact on an isobutanol producing microorganism. Conversion of one mole of glucose to two moles of pyruvate via glycolysis leads to the production of two moles of NADH. A metabolic pathway that converts pyruvate to a target product that consumes either two moles of NADPH or one mole of NADH and one mole of NADPH leads to cofactor imbalance that results in (a) the cell's inability to produce isobutanol at theoretical yield, and (b) the cell's inability to produce isobutanol under anaerobic conditions. In conjunction with the use of an NADH-dependent alcohol dehydrogenase enzyme (ADH), the S. exigua KARI or an NKR derivative thereof may help resolve these problems. Third, the S. exigua KARI or an NKR derivative thereof may be useful in the context of other biosynthetic pathways comprising KARI enzymes. Such metabolites, include, but are not limited to, isoleucine, leucine, valine, pantothenate, coenzyme A, 1-butanol, 2-methyl-1-butanol, 3-methyl-1-butanol, 3-methyl-1-pentanol, 4-methyl-1-pentanol, 4-methyl-1-hexanol, and 5-methyl-1-heptanol.

### Example 5

### The S. exigua KARI Exhibits Reduced Product Inhibition

The purpose of this example is to show that the S. exigua KARI exhibits a high product inhibition constant (Kᵢ) and a high ratio of product inhibition constant to Michaelis-Menten constant (K_{M}). Unlike the S. exigua KARI, most KARI enzymes found in nature generally exhibit low K values and low Kᵢ/K_{M} values. The S. exigua KARI disclosed herein appears to be fundamentally different (better) with respect to product inhibition by DHIV. This example also shows that the S. exigua KARI exhibits a low K_{M} for Mg²⁺, acetolactate and cofactor. Unlike the S. exigua KARI, most KARI enzymes found in nature generally exhibit higher K_{M} values. The S. exigua KARI disclosed herein appears to be fundamentally different (better) with respect to affinity towards substrate (S)-2-acetolactate and cofactors (NADH, NADPH, Mg²⁺). This example also shows that the S. exigua KARI exhibits a low optimum pH. Unlike the S. exigua KARI, most KARI enzymes found in nature generally exhibit higher pH optima. The S. exigua KARI disclosed herein appears to be fundamentally different (better) with respect to pH optimum.

### Materials and Methods for Example 5

TABLE 9
Strain Used in Example 5.

| Strain/Organism | Genotype |
|---|---|
| E. coli BL21(DE3) | F⁻ ompT gal dcm lon hsdS_{B}(r_{B}⁻ m_{B}⁻) λ(DE3 |
| | [lacl lacUV5-T7 gene 1 ind1 sam7 nin5] |

TABLE 10
Plasmids Used in Example 5.

| Plasmids | Genotype |
|---|---|
| pET22b(+) | PT7, bla, ori pBR322, lacl, C-term 6xHis |
| pET[ilvC] | PT7::Ec_ilvC_coEc^{his6}, bla, oripBR322, lacl |
| pET3002 | PT7::Se1_KARI_coSc^{his6}, bla, oripBR322, lacl |
| pGV3281 | PT7::Se1_KARI_coSc^{his6}, bla, oripBR322, lacl |

Heterologous Expression of S. exigua KARI in E. coli:

Expression of the S. exigua KARI was conducted in a 2-L baffled Erlenmeyer flask filled with 1 L LBₐₘₚ (Luria Bertani Broth, Research Products International Corp, supplemented with 100 μg/mL ampicillin) inoculated with overnight culture to an initial OD₆₀₀ of 0.1. After growing the expression culture at 37° C. with shaking at 250 rpm for 4 h, the cultivation temperature was dropped to 25° C., and KARI expression was induced with IPTG to a final concentration of 0.5 mM. After 24 h at 25° C. and shaking at 250 rpm, the cells were pelleted at 5,300 g for 10 min and then frozen at −20° C. until further use.

Synthesis of Enantiopure S-2-Acetolactate:

Enzymatic synthesis of (S)-2-acetolactate was performed in an anaerobic flask. The reaction was carried out in a total volume of 55 mL containing 20 mM potassium phosphate buffer, pH 7.0, 1 mM MgCl₂, 0.05 mM thiamine pyrophosphate (TPP), and 200 mM sodium pyruvate. The synthesis was initiated by the addition of 65 units of purified B. subtilis acetolactate synthase (Bs_AlsS), and the reaction was incubated at 30° C. (in a static incubator) for 7.5 hours. A buffer exchange was performed on the purified Bs_AlsS before the synthesis to remove glycerol. This was done using a microcon filter with a 50 kDa nominal molecular weight cutoff membrane to filter 0.5 mL of the purified enzyme until only 50 μL were left on top of the membrane. 450 μL of 20 mM KPO₄ pH 7.0, 1 mM MgCl₂, and 0.05 mM TPP were then added to the membrane and filtered again, this process was repeated 3 times. The final acetolactate concentration was determined by HPLC and was 200 mM in this batch used here.

KARI Assay in 1-mL Scale to Measure NADPH and NADH K_{M} Values:

KARI activity was assayed kinetically by monitoring the decrease in NADPH or NADH concentration by measuring the change in absorbance at 340 nm. An assay buffer was prepared containing 100 mM potassium phosphate pH 7.0, 1 mM DTT, 2.5 mM (S)-2-acetolactate, and 10 mM MgCl₂ (final concentrations in the 1-mL assay). Twenty μL purified enzyme and 960 μL of the assay buffer were placed into a 1-mL cuvette. The reaction was initiated by addition of 20 μL NADPH or NADH (200 μM final concentration) for a general activity assay. Michaelis-Menten constants for the cofactors were determined with varying concentrations of NADPH or NADH (200-12 μM final).

KARI Assay in 100-μL Scale to Measure MgCl₂ K_{M} Value:

An assay buffer was prepared containing 100 mM potassium phosphate pH 7.0, 1 mM DTT, 2.5 mM (S)-2-acetolactate, and 200 μM NADPH (final concentrations). Five μL purified enzyme and 5 μL of varying concentrations of MgCl₂ were placed into a flat-bottom assay plate (Evergreen Scientific). The reactions were initiated by addition of 90 μL assay buffer. The change in absorbance at 340 nm was monitored over 1 min in a plate reader (TECAN).

KARI Assay in 100-μL Scale to Measure Acetolactate K_{M} Value:

An assay buffer was prepared containing 100 mM potassium phosphate pH 7.0, 1 mM DTT, 10 mM MgCl₂, and 200 μM NADPH (final concentrations). Five μL purified enzyme and 5 μL of varying concentrations of acetolactate (8-0.125 mM final) were placed into a flat-bottom assay plate (Evergreen Scientific). The reactions were initiated by addition of 90 μL assay buffer. The change in absorbance at 340 nm was monitored over 1 min in a plate reader (TECAN).

KARI Assay in 100-μL Scale to Measure pH Optimum:

Assay buffers were prepared containing 100 mM potassium phosphate with pH values ranging from 6.0-8.5 in 0.5-steps, 1 mM DTT, 10 mM MgCl₂, 2.5 mM acetolactate, and 200 μM NADPH (final concentrations). Ten μL purified enzyme were placed into a flat-bottom assay plate (Evergreen Scientific). The reactions were initiated by addition of 90 μL assay buffer. The change in absorbance at 340 nm was monitored over 1 min in a plate reader (TECAN).

KARI Assay in 100-μL Scale to Measure IC₅₀ in Presence of R-DHIV:

Assay buffers were prepared containing 250 mM potassium phosphate pH 7.0, 1 mM DTT, 10 mM MgCl₂, 2.5 mM acetolactate, 200 μM NADPH, and increasing concentrations of R-DHIV (0-85.5 mM (final concentrations). Ten μL purified enzyme were placed into a flat-bottom assay plate (Evergreen Scientific). The reactions were initiated by addition of 90 μL assay buffer. The change in absorbance at 340 nm was monitored over 1 min in a plate reader (TECAN).

Results:

Purification of Enzymes and Comparison of S. exigua KARI to L. lactis KARI and E. coli KARI:

All three KARIs were expressed and purified under the same conditions (see material and methods section). After purification, the purity of the enzymes was determined using an SDS gel. 70 μg of the E. coli KARI, 30 μg of the L. lactis KARI, and 35 μg of S. exigua KARI were loaded. The proteins were >90% pure after the nickel column. The proteins were characterized for their kinetic properties. The results are summarized in Table 11.

NADPH K_{M} Value:

The K_{M} value of the S. exigua KARI for NADPH was measured as described in the Material and Methods section. However, it was not possible to determine the K_{M} value exactly, since the enzyme was still saturated when the NADPH detection limit in the spectrophotometer was reached. The estimated K_{M} value for NADPH is most likely below 1 μM. The NADPH K_{M} for E. coli KARI was likely 20 times higher and the NADPH K_{M} for L. lactis KARI was 13 fold higher than the NADPH K_{M} for S. exigua KARI

NADH K_{M} Value:

The K_{M} value of the S. exigua KARI for NADH was measured as described in the Material and Methods section. The NADH K_{M} value was determined at 45±10 μM. The NADH K_{M} for E. coli KARI was 22 times higher and the NADH K_{M} for L. lactis KARI was 6 fold higher than the NADH K_{M} for S. exigua KARI. The k_{cat} with NADH was calculated and is 0.4 s⁻¹.

Acetolactate K_{M} Value:

The K_{M} value of the S. exigua KARI for acetolactate was measured as described above and determined to be 0.17 mM±0.01. The K_{M} for acetolactate measured for E. coli KARI and L. lactis KARI were 32 times and 1.8 times higher than the K_{M} value of the S. exigua KARI for acetolactate.

Mg²⁺ K_{M} Value:

The K_{M} value of the S. exigua KARI for Mg²⁺ was measured as described above and determined to be 0.7 mM±0.08. The K_{M} for Mg²⁺ measured for E. coli KARI and L. lactis KARI were 2.9 times and 6.9 times higher than the K_{M} value of the S. exigua KARI for Mg²⁺.

R-DHIV Inhibition Described as IC₅₀ Value and as Calculated Kᵢ:

The inhibition of the S. exigua KARI with the product R-DHIV was measured as described above. The E. coli KARI was used as a positive control, since making R-DHIV solutions of accurate concentrations is complicated by the highly viscous nature of the material. The E. coli KARI IC₅₀ was 1 mM±0.1. In contrast, the IC₅₀ of the S. exigua KARI was dramatically higher (26.7 mM±7.5). Using the IC₅₀ value, the Kᵢ was calculated to be 1.7 mM, and 17 times higher than the K, calculated for E. coli KARI and similar to the Kᵢ calculated for a KARI from Lactococcus lactis (Table 11).

DH Optimum:

The optimum pH of S. exigua KARI was measured as described above and the result is shown in FIG. 3. The optimal pH was determined to be approximately 7.0. This pH optimum was lower than the pH optimum for L. lactis and for E. coli KARI (Table 11).

TABLE 11
Summary of Analysis For Three Analyzed KARIs

| Parameter | Se1_KARI | Ll_KARI | E. coli IlvC |
|---|---|---|---|
| IC₅₀ [mM]^{a} | 27 ± 7.5 | 2.5 ± 0.7 | 1.1 ± 0.1 |
| Kᵢ [mM]^{a, b} | 1.7 | 1.7 | 0.1 |
| optimum pH | 7 | ≧8.5 | 7.5 |
| K_{M} Mg²⁺ [mM] | 1.7 ± 0.08 | 4.8 ± 0.9 | 2 ± 0.5 |
| K_{M} (S)-2-acetolactate [mM] | 0.17 ± 0.01 | 5.6 ± 1.6 | 0.3 ± 0.1 |
| K_{M} NADPH [μM] | n.d.^{c} | 13 ± 1.3 | 20 ± 3 |
| K_{M} NADH [μM] | 45 ± 10 | 285 ± 30 | 1000 |

^{a}For (R)-DHIV
^{b}Calculated via Cheng-Prusoff equation
^{c}Not measurable because the enzyme was still saturated at the lowest detectable NADPH concentration of 12 μM. Estimated to be lower than 1 μM.
(Se1 = S. exigua;
Ll = L. lactis).

Conclusions:

(1) The NADH K_{M} value of the S. exigua KARI is very low (45 μM) compared to the E. coli KARI (˜1000 μM); (2) The IC₅₀ of the S. exigua KARI measured in the presence of 2.5 mM (R)-DHIV of 27 mM, is 10 times higher than the IC₅₀ of the L. lactis KARI and 27 times higher than the IC₅₀ of the E. coli KARI; (3) The K_{M} of the S. exigua KARI is 10-fold lower than the Kᵢ for R-DHIV, while for the L. lactis KARI, the K_{M} is 3.3 fold higher than the K, and for the E. coli KARI the K_{M} is 3 fold higher than the K; and (4) The S. exigua KARI has the lowest substrate K_{M} value of the three KARIs tested.

### Example 6

### Cofactor Switch of the S. exigua KARI

The purpose of this example is to demonstrate how the cofactor specificity of the S. exigua KARI can be switched from NADPH to NADH.

Similar to all known native KARI enzymes, the S. exigua KARI is NADPH-dependent. To enable the enzyme's use in the production of isobutanol at theoretical yield and/or under anaerobic conditions, the enzyme's cofactor usage was switched from NADPH to NADH.

### Materials and Methods for Example 6

TABLE 12
Strains Used in Example 6.

| Strain | Genotype/Source |
|---|---|
| E. coli | F⁻ ompT gal dcm lon hsdS_{B}(r_{B}⁻ m_{B}⁻) λ(DE3 |
| BL21(DE3) | [lacl lacUV5-T7 gene 1 ind1 sam7 nin5] |

TABLE 13
Plasmids Used in Example 6.

| Plasmid | Genotype |
|---|---|
| pET22b(+) | PT7, bla, ori pBR322, lacl, C-term 6xHis |
| pET[ilvC] | PT7::Ec_ilvC_coEc^{his6}, bla, oripBR322, lacl |
| pGV3195 | PT7::Se1_KARI_coSc^{his6}, bla, oripBR322, lacl |
| pETSe1LE1 | PT7::Se1_KARI^{LE1}_coSc^{his6}, bla, oripBR322, lacl |
| pETSe1GE11 | PT7::Se1_KARI^{GE11}_coSc^{his6}, bla, oripBR322, lacl |
| pETSe1HF2 | PT7::Se1_KARI^{HF2}_coSc^{his6}, bla, oripBR322, lacl |
| pETSe11H1 | PT7::Se1_KARI^{1H1}_coSc^{his6}, bla, oripBR322, lacl |
| pETSe12C6 | PT7::Se1_KARI^{2C6}_coSc^{his6}, bla, oripBR322, lacl |
| pETSe14E10 | PT7::Se1_KARI^{4E10}_coSc^{his6}, bla, oripBR322, lacl |
| pETSe11A8 | PT7::Se1_KARI^{1A8}_coSc^{his6}, bla, oripBR322, lacl |
| pETSe113G6 | PT7::Se1_KARI^{13G6}_coSc^{his6}, bla, oripBR322, lacl |
| pETSe114B7 | PT7::Se1_KARI^{14B7}_coSc^{his6}, bla, oripBR322, lacl |
| pETSe1AC1 | PT7::Se1_KARI^{AC1}_coSc^{his6}, bla, oripBR322, lacl |
| pETSe11AA10 | PT7::Se1_KARI^{1AA10}_coSc^{his6}, bla, oripBR322, lacl |

TABLE 14
Primers Used in Examples 6-9.

| Pair # | Primer name | Sequence |
|---|---|---|
| 1 | T7_for | TAATACGACTCACTATAGGG (SEQ ID NO: 35) |
| 2 | T7_rev | GCTAGTTATTGCTCAGCGG (SEQ ID NO: 36) |
| 3 | Se_I33NNK_for | GGTCTTAAAGTTGGTATCNNKGGTTACGGTTCCC |
| | | AAGGT (SEQ ID NO: 37) |
| 4 | Se_I33NNK_rev | ACCTTGGGAACCGTAACCMNNGATACCAACTTTA |
| | | AGACC (SEQ ID NO: 38) |
| 5 | Se_G34NNK_for | AAAGTTGGTATCATCNNKTACGGTTCCCAAGGT |
| | | (SEQ ID NO: 39) |
| 6 | Se_G34NNK_rev | ACCTTGGGAACCGTAMNNGATGATACCAACTTT |
| | | (SEQ ID NO: 40) |
| 7 | Se_Y35NNK_for | GTTGGTATCATCGGTNNKGGTTCCCAAGGTCAC |
| | | (SEQ ID NO: 41) |
| 8 | Se_Y35NNK_rev | GTGACCTTGGGAACCMNNACCGATGATACCAAC |
| | | (SEQ ID NO: 42) |
| 9 | Se_G36NNK_for | GGTATCATCGGTTACNNKTCCCAAGGTCACGCT |
| | | (SEQ ID NO: 43) |
| 10 | Se_G36NNK_rev | AGCGTGACCTTGGGAMNNGTAACCGATGATACC |
| | | (SEQ ID NO: 44) |
| 11 | Se_L57NNK_for | GATGTTAGAGTTGGCNNKAGAGAAGGCTCATCT |
| | | (SEQ ID NO: 45) |
| 12 | Se_L57NNK_rev | AGATGAGCCTTCTCTMNNGCCAACTCTAACATC |
| | | (SEQ ID NO: 46) |
| 13 | Se_R58NNK_for | GTTAGAGTTGGCTTANNKGAAGGCTCATCTAGT |
| | | (SEQ ID NO: 47) |
| 14 | Se_R58NNK_rev | ACTAGATGAGCCTTCMNNTAAGCCAACTCTAAC |
| | | (SEQ ID NO: 48) |
| 15 | Se_G60NNK_for | GTTGGCTTAAGAGAANNKTCATCTAGTTGGAAA |
| | | (SEQ ID NO: 49) |
| 16 | Se_G60NNK_rev | TTTCCAACTAGATGAMNNTTCTCTTAAGCCAAC |
| | | (SEQ ID NO: 50) |
| 17 | Se_S61NNK_for | GGCTTAAGAGAAGGCNNKTCTAGTTGGAAAACG |
| | | (SEQ ID NO: 51) |
| 18 | Se_S61NNK_rev | CGTTTTCCAACTAGAMNNGCCTTCTCTTAAGCC |
| | | (SEQ ID NO: 52) |
| 19 | Se_S62NNK_for | TTAAGAGAAGGCTCANNKAGTTGGAAAACGGCT |
| | | (SEQ ID NO: 53) |
| 20 | Se_S62NNK_rev | AGCCGTTTTCCAACTMNNTGAGCCTTCTCTTAA |
| | | (SEQ ID NO: 54) |
| 21 | Se_S63NNK_for | AGAGAAGGCTCATCTNNKTGGAAAACGGCTGAG |
| | | (SEQ ID NO: 55) |
| 22 | Se_S63NNK_rev | CTCAGCCGTTTTCCAMNNAGATGAGCCTTCTCT |
| | | (SEQ ID NO: 56) |
| 23 | Se_L90NNK_for | GATGTCATCATGGTTNNKGTGCCTGATGAAATT |
| | | (SEQ ID NO: 57) |
| 24 | Se_L90NNK_rev | AATTTCATCAGGCACMNNAACCATGATGACATC |
| | | SEQ ID NO: 58) |
| 25 | Se_I95NNK_for | TTGGTGCCTGATGAANNKCAACCTAAGGTATAT |
| | | (SEQ ID NO: 59) |
| 26 | Se_I95NNK_rev | ATATACCTTAGGTTGMNNTTCATCAGGCACCAA |
| | | (SEQ ID NO: 60) |
| 27 | Se_V99NNK_for | GAAATTCAACCTAAGNNKTATCAGGAACATATC |
| | | (SEQ ID NO: 61) |
| 28 | Se_V99NNK_rev | GATATGTTCCTGATAMNNCTTAGGTTGAATTTC |
| | | (SEQ ID NO: 62) |
| 29 | Se_recomb1_Y35YAC_for | GGTATCATCGGTYACGGTTCCCAAGGT |
| | | (SEQ ID NO:63) |
| 30 | Se_recomb1_Y35YAC_rev | ACCTTGGGAACCGTRACCGATGATACC |
| | | (SEQ ID NO: 64) |
| 31 | Se_recomb2a_for | GGCTTAAGAGAAGKATSCTCTAGTTGGAAAACGGCT |
| | | (SEQ ID NO: 65) |
| 32 | Se_recomb2b_for | GGCTTAAGAGAAGKATSCTCTGATTGGAAAACGGCT |
| | | (SEQ ID NO: 66) |
| 33 | Se_recomb2c_for | GGCTTAAGAGAAGKATSCTCTCAGTGGAAAACGGCT |
| | | (SEQ ID NO: 67) |
| 34 | Se_recomb2a_rev | AGCCGTTTTCCAACTAGAGSATMCTTCTCTTAAGCC |
| | | (SEQ ID NO: 68) |
| 35 | Se_recomb2b_rev | AGCCGTTTTCCAATCAGAGSATMCTTCTCTTAAGCC |
| | | (SEQ ID NO: 69) |
| 36 | Se_recomb2c_rev | AGCCGTTTTCCACTGAGAGSATMCTTCTCTTAAGCC |
| | | (SEQ ID NO: 70) |
| 37 | Se_recomb3a_for | CCTGATGAAAHCCAACCTAAGKTATATCAGGAA |
| | | (SEQ ID NO: 71) |
| 38 | Se_recomb3b_for | CCTGATGAAGYACAACCTAAGKTATATCAGGAA |
| | | (SEQ ID NO: 72) |
| 39 | Se_recomb3a_rev | TTCCTGATATAMCTTAGGTTGGDTTTCATCAGG |
| | | (SEQ ID NO: 73) |
| 40 | Se_recomb3b_rev | TTCCTGATATAMCTTAGGTTGTRCTTCATCAGG |
| | | (SEQ ID NO: 74) |
| 41 | Se_recomb4a_L57TYA_for | GTCGATGTTAGAGTTGGCTYAAGAGAA |
| | | (SEQ ID NO: 75) |
| 42 | Se_recomb4a_L57TYA_rev | TTCTCTTRAGCCAACTCTAACATCGAC |
| | | (SEQ ID NO: 76) |
| 43 | Se_recomb4b_L57CRA_for | GTCGATGTTAGAGTTGGCCRAAGAGAA |
| | | (SEQ ID NO: 77) |
| 44 | Se_recomb4b_L57CRA_rev | TTCTCTTYGGCCAACTCTAACATCGAC |
| | | (SEQ ID NO: 78) |
| 45 | Se_L57NNK_R58P_S61TSC_for | AGAGTTGGCNNKCCAGAAGGCTSCTCTAGTTGG |
| | | (SEQ ID NO: 79) |
| 46 | Se_L57NNK_R58P_S61TSC_rev | CCAACTAGAGSAGCCTTCTGGMNNGCCAACTCT |
| | | (SEQ ID NO: 80) |
| 47 | Serec4_fancy3_rec_A_for | GTTAGAGTTGGCGTACCAGAAGKATGCTCTAGTT |
| | | GGAAA (SEQ ID NO: 81) |
| 48 | Serec4_fancy3_rec_A_rev | TTTCCAACTAGAGCATMCTTCTGGTACGCCAACT |
| | | CTAAC (SEQ ID NO: 82) |
| 49 | Serec4_fancy3_rec_B_for | CCTGATGAARYACAACCTAAGKTATATCAGGAACAT |
| | | (SEQ ID NO: 83) |
| 50 | Serec4_fancy3_rec_B_rev | ATGTTCCTGATATAMCTTAGGTTGTRYTTCATCAGG |
| | | (SEQ ID NO: 84) |
| 51 | Se1_S63D_S61D_for | GGCTTAAGAGAAGGCGACTCTGACTGGAAAACG |
| | | GCTGAG (SEQ ID NO: 85) |
| 52 | Se1_S63D_S61D_rev | CTCAGCCGTTTTCCAGTCAGAGTCGCCTTCTCTT |
| | | AAGCC (SEQ ID NO: 86) |
| 53 | SeAA10R58S62NNK_for | GTTGGCTTANNKGAAGGCTGCNNKGATTGGAAA |
| | | ACGGCT (SEQ ID NO: 87) |
| 54 | SeAA10R58S62NNK_rev | AGCCGTTTTCCAATCMNNGCAGCCTTCMNNTAA |
| | | GCCAAC (SEQ ID NO: 88) |

* A (Adenine),
G (Guanine),
C (Cytosine),
T (Thymine),
R (Purine - A or G),
Y (Pyrimidine - C or T),
N (Any nucleotide),
S (Strong - G or C),
M (Amino - A or C),
K (Keto - G or T),
H (Not G - A or C or T), and
D (Not C - A or G or T)

Heterologous Expression of Wild-Type S. exigua KARI in E. coli:

Expression of wild-type S. exigua KARI was conducted in a 2-L baffled Erlenmeyer flask filled with 1 L LBₐₘₚ (Luria Bertani Broth, Research Products International Corp, supplemented with 100 μg/mL ampicillin) inoculated with overnight culture to an initial OD₆₀₀ of 0.1. After growing the expression culture at 37° C. with shaking at 250 rpm for 4 h, the cultivation temperature was dropped to 25° C., and KARI expression was induced with IPTG to a final concentration of 0.5 mM. After 24 h at 25° C. and shaking at 250 rpm, the cells were pelleted at 5,300 g for 10 min and then frozen at −20° C. until further use.

Heterologous Expression of S. exigua KARI Variants in E. coli:

The expression of S. exigua KARI variants was conducted in 0.25-L Erlenmeyer flasks filled with 50 mL LBₐₘₚ (Luria Bertani Broth, Research Products International Corp, supplemented with 100 μg/mL ampicillin) inoculated with overnight culture to an initial OD₆₀₀ of 0.1. After growing the expression cultures at 37° C. with shaking at 250 rpm for 4 h, the cultivation temperature was dropped to 25° C., and KARI expression was induced with IPTG to a final concentration of 0.5 mM. After 24 h at 25° C. and shaking at 250 rpm, the cells were pelleted at 5,300 g for 10 min and then frozen at −20° C. until further use.

Histrap Purification of S. exigua KARI:

S. exigua KARI was purified over a 5-mL histrap column.

Histrap Purification of S. exigua KARI Variants:

S. exigua KARI variants were purified over 1-mL histrap columns.

Preparation of Enantiopure (S)-2-Acetolactate:

Enzymatic synthesis of (S)-2-acetolactate was performed in an anaerobic flask. The reaction was carried out in a total volume of 55 mL containing 20 mM potassium phosphate buffer, pH 7.0, 1 mM MgCl₂, 0.05 mM thiamine pyrophosphate (TPP), and 200 mM sodium pyruvate. The synthesis was initiated by the addition of 65 units of purified B. subtilis acetolactate synthase (Bs_AlsS), and the reaction was incubated at 30° C. (in a static incubator) for 7.5 hours. A buffer exchange was performed on the purified Bs_AlsS before the synthesis to remove as much glycerol as possible. This was done using a microcon filter with a 50 kDa nominal molecular weight cutoff membrane to filter 0.5 mL of the purified enzyme until only 50 μL were left on top of the membrane. 450 μL of 20 mM KPO₄ pH 7.0, 1 mM MgCl₂, and 0.05 mM TPP were then added to the membrane and filtered again; this process was repeated three times. The final acetolactate concentration was determined by liquid chromatography and was ˜200 mM.

KARI Assay in 1-mL Scale to Measure NADPH and NADH K_{M} Values:

S. exigua KARI activity or activities of its variants were assayed kinetically by monitoring the decrease in NADPH or NADH concentration by measuring the change in absorbance at 340 nm. An assay buffer was prepared containing 100 mM potassium phosphate pH 7.0, 1 mM DTT, 2.5 mM (S)-2-acetolactate, and 10 mM MgCl₂ (final concentrations in the 1-mL assay, accounting for dilution with enzyme and cofactor). Fifty μL purified enzyme and 930 μL of the assay buffer were placed into a 1-mL cuvette. The reaction was initiated by addition of 20 μL NADPH or NADH (200 μM final concentration) for a general activity assay. Michaelis-Menten constants of the cofactors were determined with varying concentrations of NADPH (200-12 μM final) or NADH (200-6 μM final).

Construction of Site-Saturation Libraries (Generation 1):

Thirteen one-site site-saturation libraries (with NNK codons) were constructed using standard SOE PCR with Phusion polymerase, pGV3195 as template, and respective primer pair numbers 1-28. The fragments were DpnI digested for 1 h, separated on an agarose gel, freeze'n'squeeze (BIORAD) treated, and finally precipitated with pellet paint (Novagen). The clean fragments served as templates for the assembly PCRs using commercial T7 forward and reverse primers (primers #1 and 2) as flanking primers. After successful assembly, the insert was restriction digested with NdeI and XhoI, ligated into pET22b(+), and electro-competent BL21(D3) cells (Lucigen) were transformed.

Construction of Recombination Library (Generation 2a):

The recombination library was constructed using SOE PCR introducing mutations found at the 13 target sites while allowing for the respective wild-type residues as well. Using pGV3195 as template and primers #1, 2, and 29-44, four fragments were generated. Primers 31 through 44, with 31-36, 37-40, and 41-44 as the respective forward and reverse pairs for fragment generation, were mixed manually to give equimolar distributions of the mutations they contained. The fragments were DpnI-digested for 1 h at 37° C., separated them on an agarose gel, freeze'n'squeezed (BIORAD) and finally pellet painted them (Novagen). The fragments served as templates in the assembly PCR using commercial T7 forward and reverse primers as flanking primers. The purified assembly product (Zymo clean up) was restriction digested with NdeI and XhoI, ligated into pET22b(+), and electro-competent BL21(D3) cells (Lucigen) were transformed.

High-throughput expression of S. exigua KARI Variants in E. coli:

For growth and expression of KARI variants in deep well plates, sterile toothpicks were used to pick single colonies into shallow 96-well plates filled with 300 μL LBₐₘₚ. Fifty μL of these overnight cultures were used to inoculate deep well plates filled with 600 μL of LBₐₘₚ per well. The plates were grown at 37° C. with shaking at 250 rpm for 3 h. One hour before induction with IPTG (final concentration 0.5 mM), the temperature of the incubator was reduced to 25° C. After induction, growth and expression continued for 20 h at 25° C. and 250 rpm. Cells were harvested at 5,300 g and 4° C. and then stored at −20=C. The plates always contained four wild-type or parent S. exigua KARI colonies, three BL21(DE3) colonies carrying pET22b(+) to control for background reactions in cell lysates, and one well that contained only media to make sure the plates were free of contaminations.

High-Throughput Screening:

Frozen cell pellets were thawed at room temperature for 20 min and then 200 μL of lysis buffer (100 mM Kpi, 750 mg/L lysozyme, 10 mg/L DNaseI, pH 7) were added. Plates were vortexed to resuspend the cell pellets. After a 60 min incubation phase at 37° C. and shaking at 130 rpm, plates were centrifuged at 5,300 g and 4° C. for 10 min. Forty μL of the resulting crude extracts were transferred into assay plates (flat bottom, Rainin) using a liquid handling robot. Twenty mL assay buffer per plate were prepared (100 mM Kpi, pH 7, 2.5 mM (S)-2-acetolactate, 1 mM DTT, 200 μM NADPH or NADH, and 10 mM MgCl₂) and 160 μL thereof were added to each well to start the reaction resulting in a 20% dilution of the ingredients. The depletion of NAD(P)H was monitored at 340 nm in a plate reader (TECAN) over 200 s.

The residues chosen to test by site-saturation mutagenesis were I33, G34, Y35, G36, L57, R58, G60, S61, S62, S63, L90, 195, and V99 of the S. exigua KARI (SEQ ID NO: 2). Site-saturation libraries were constructed as described in the materials and method section. After successful transformation of BL21(DE3) cells, 88 individual clones per library were chosen. The libraries were screened with NADH (not NADPH) as cofactor. After sequencing 23 variants from all libraries, potential variants from libraries I33, G34, G36, R58, S62, and L90 were eliminated. Screening results are summarized in Table 15.

TABLE 15
Exemplary Variants of Generation 1 NNK Libraries.

| | | | | | Ratio |
|---|---|---|---|---|---|
| | S. exigua | Beneficial | U/mg | U/mg | NADH/ |
| Variant name | KARI | mutations | NADH | NADPH | NADPH |
| Se1_KARI^{his6} | Parent | — | 0.15 | 0.32 | 0.47 |
| Se1_KARI^{CD10-his6} | Y35 | H | 0.13 | 0.19 | 0.68 |
| Se1_KARI^{EA8-his6} | L57 | S | 0.19 | 0.25 | 0.76 |
| Se1_KARI^{EE1-his6} | | R | 0.06 | 0.04 | 1.58 |
| Se1_KARI^{GE11-his6} | G60 | V | 0.4 | 0.8 | 0.48 |
| Se1_KARI^{HF2-his6} | S61 | C | 0.18 | 0.23 | 0.78 |
| Se1_KARI^{JB7-his6} | S63 | Q | 0.28 | 0.6 | 0.47 |
| Se1_KARI^{LC1-his6} | I95 | A | 0.13 | 0.17 | 0.76 |
| Se1_KARI^{LE9-his6} | | T | 0.32 | 0.56 | 0.58 |
| Se1_KARI^{LE1-his6} | | V | 0.3 | 0.4 | 0.8 |
| Se1_KARI^{LD1-his6} | | N | 0.12 | 0.15 | 0.86 |
| Se1_KARI^{MH9-his6} | V99 | L | 0.21 | 0.34 | 0.64 |

Residue S63 in the S. exigua KARI corresponds to residue S78 in the wild-type E. coli KARI.

Recombination Library (Generation 2a):

A recombination library introducing all mutations identified at each targeted site while also allowing for the wild-type residues was constructed. In addition, the S63D mutation was also included. 2,000 clones were screened with NADPH and NADH. 33 clones were rescreened. Six were chosen for sequencing and characterization. The six remaining clones showed at least doubled NADH/NADPH activity ratios in the rescreen.

Five variants (1A8, 13G6, 14B7, AC1, and 1AA10) were expressed, purified, and characterized (Table 16). Variant six was the single mutant 195V identified in Generation 1. All five variants were mutated at residue 95, with valine and alanine each showing up twice, and threonine showing up once in variant 1AA10. Three variants, 14B7, AC1, and 1AA10, contained the S63D mutation, but only 1AA10 was found to have a switch in cofactor specificity. It is the only variant which also carries a mutation at position S61 (S61C). The NADPH K_{M} value was at least 367-fold increased; the NADH K_{M} value was 1.3-fold increased. Variant 1AA10 has parent-like activity (in U/mg) on NADH, with 0.8 U/mg, and a parent-like k_{cat} value with NADH (0.8 s⁻¹).

TABLE 16
Comparison of S. exigua KARI and Variants Thereof.

| | Variant | Mutations | | | | | | | | U/mg | | |
|---|---|---|---|---|---|---|---|---|---|---|---|---|
| Gen | (gene) | Y35 | L57 | R58 | G60 | S61 | S63 | I95 | V99 | NADH | NADPH | ratio |
| 0 | Se1_KARI | Y | L | R | G | S | S | I | V | 0.15 | 0.32 ± 0.03 | 0.47 |
| 1 | Se1_KARI^{LE1} | | | | | | | V | | 0.30 | 0.40 | 0.8 |
| 1 | Se1_KARI^{GE11} | | | | V | | | | | 0.40 | 0.8 | 0.50 |
| 1 | Se1_KARI^{HF2} | | | | | C | | | | 0.18 | 0.23 | 0.78 |
| 1 | Se1_KARI^{JB7} | | | | | | Q | | | 0.28 | 0.6 | 0.47 |
| 2a | Se1_KARI^{1A8} | | | | V | | | V | L | 0.26 | 0.32 | 0.8 |
| 2a | Se1_KARI^{13G6} | | | | V | | | A | | 0.30 | 0.37 | 0.8 |
| 2a | Se1_KARI^{14B7} | H | | | | | D | V | | 0.18 | 0.56 | 0.32 |
| 2a | Se1_KARI^{AC1} | | | | | | D | A | | 0.2 | 0.4 | 0.5 |
| 2a | Se1_KARI^{1AA10} | | | | | C | D | T | | 0.32 | 0.27 | 1.2 |
| | | | | | | Kₘ [μM] for | | | | | | |
| | | | | | | cofactor | | k_{cat} [^{s-1}] | | k_{cat}/Kₘ [M⁻¹*s⁻¹] | | |
| | | | | | Gen | NADH | NADPH | NADH | NADPH | NADH | NADPH | ratio |
| | | | | | 0 | 45 ± 10 | <1 | 0.40 | 0.80 | 8,889 | >800,000 | 0.0 |
| | | | | | 1 | 19 | n/a* | 0.74 | 1.00 | | | |
| | | | | | 1 | n/a | n/a | 1 | 2 | | | |
| | | | | | 1 | 43.8 | n/a | 0.45 | 0.57 | | | |
| | | | | | 1 | n/a | n/a | 0.69 | 1.5 | | | |
| | | | | | 2a | n/a | n/a | 0.65 | 0.79 | | | |
| | | | | | 2a | 34 | <1 | 0.74 | 0.90 | 21,765 | >800,000 | 0.03 |
| | | | | | 2a | n/a | n/a | 0.45 | 1.4 | | | |
| | | | | | 2a | 68 | 45 | 0.5 | 1 | 7,353 | 22,222 | 0.3 |
| | | | | | 2a | 62 | 367 | 0.8 | 0.67 | 12,903 | 1,826 | 7.1 |

L57NNKR58PS61 S/C-Library (Generation 2b) and Recombination Library (Generation 2c) of Variant 4E10 with Gen2a Variant 1A8:

A focused library was built in parallel to the recombination library Gen2a. Residues L57, R58, and S61 were pinpointed as most likely candidates involved in binding. Both S and C in the Generation 2b library was allowed at position 61. Potential binding to R58 was disrupted by replacing it with a proline, which appeared to be least disruptive for folding. This allowed for the targeting of L57 with site-saturation mutagenesis. The benefit of generating a focused library, instead of generating a double NNK library, was the library size.

After screening four 96-well plates for the consumption of NADH and NADPH, 18 were rescreened and three variants were identified (Table 17), which showed almost doubled (and one, 4E10, more than doubled) NADH/NADPH ratios in the screen. After purification, variant 4E10 had a ratio of 1 in U/mg, being the only triple mutant with mutations L57V, R58P, and S61C. The improved ratio, however, did not result from decreased NADPH activity, which in fact was slightly improved, but from more than doubled activity on NADH. This supports the suggestion that another phosphate-binding residue, likely S63D, is still available to bind phosphate.

TABLE 17
Variants Found in L57NNKR58PS61S/C-library (Generation 2b).

| | Variant | Mutations | | | | | | | | U/mg | | |
|---|---|---|---|---|---|---|---|---|---|---|---|---|
| Gen | (gene) | Y35 | L57 | R58 | G60 | S61 | S63 | I95 | V99 | NADH | NADPH | ratio |
| 0 | Se1_KARI | Y | L | R | G | S | S | I | V | 0.15 | 0.32 ± 0.03 | 0.47 |
| 1 | Se1_KARI^{LE1} | | | | | | | V | | 0.30 | 0.40 | 0.8 |
| 1 | Se1_KARI^{GE11} | | | | V | | | | | 0.40 | 0.8 | 0.50 |
| 1 | Se1_KARI^{HF2} | | | | | C | | | | 0.18 | 0.23 | 0.78 |
| 1 | Se1_KARI^{JB7} | | | | | | Q | | | 0.28 | 0.6 | 0.47 |
| 2b | Se1_KARI^{1H1} | | | P | | C | | | | 0.20 | 0.26 | 0.8 |
| 2b | Se1_KARI^{2C6} | | V | P | | | | | | 0.22 | 0.33 | 0.7 |
| 2b | Se1_KARI^{4E10} | | V | P | | C | | | | 0.36 | 0.36 | 1.0 |
| | | | | | | Kₘ [μM] for | | | | | | |
| | | | | | | cofactor | | k_{cat} [^{s-1}] | | k_{cat}/Kₘ [M⁻¹*s⁻¹] | | |
| | | | | | Gen | NADH | NADPH | NADH | NADPH | NADH | NADPH | ratio |
| | | | | | 0 | 45 ± 10 | <1 | 0.40 | 0.80 | 8,889 | >800,000 | 0.0 |
| | | | | | 1 | 19 | n/a* | 0.74 | 1.00 | | | |
| | | | | | 1 | n/a | n/a | 1 | 2 | | | |
| | | | | | 1 | 43.8 | n/a | 0.45 | 0.57 | | | |
| | | | | | 1 | n/a | n/a | 0.69 | 1.5 | | | |
| | | | | | 2b | n/a | n/a | 0.50 | 0.65 | | | |
| | | | | | 2b | n/a | n/a | 0.55 | 0.80 | | | |
| | | | | | 2b | 49 | 4 | 0.90 | 0.90 | 18,367 | 225,000 | 0.1 |

Generation 2a variant Se1_KARI^{1AA10-his6} exhibits a 7-fold specificity for NADH over NADPH. The variant shows a native-like k_{cat} with NADH (0.8 s⁻¹) which corresponds to a 2-fold increase. The k_{cat} with NADPH was 1.2-fold decreased. The NADPH K_{M} value was >367-fold increased while the NADH K_{M} may have increased slightly. The 1AA10 variant carries three mutations: S61C, S63D, and 195T.

### Example 7

### Further Engineering of Slackia KARI

This example describes how the incorporation of aspartic acid mutations at positions S61 and S63 of the Slackia KARI improved NADH-dependent KARI activity.

Aspartic acid mutations were introduced into the Slackia KARI at positions S61 and S63 via “quikchange” PCR using primer pairs 51 and 52 (Table 14). Pfu turbo (Stratagene) was used as the polymerase and applied using the following PCR conditions: 95° C. for 2 mins, 95° C. for 30 secs, 55° C. for 30 secs, 72° C. for 8 mins (repeat 15 times); 72° C. for 10 mins. After the PCR program was completed, the reaction mixtures were digested with DpnI for 1 hr at 37° C. Then, chemically competent E. coli XL1-Gold cells were transformed with 3 μL of the uncleaned PCR mixtures and plated on LBₐₘₚ plates. After confirming the correct sequence, E. coli BL21(DE3) cells were transformed for expression.

The S61D/S63D variant exhibited a 10-fold higher kcat in the presence of NADH than in the presence of NADPH, with 1 s⁻¹ versus 0.1 s⁻¹. Even though the NADH K_{M} value is 2.5 times higher than the wild-type, the NADPH K_{M} is more than 800 times greater than the parent (Table 18). In conclusion, simultaneously introducing aspartic acids at positions S61 and S63 yielded a cofactor switched Slackia KARI.

Given the positive results associated with the S63D mutation, this residue was mutated to other amino acids to determine if other mutations would support the cofactor switch. S63E, S63H, S631, S63M, and S63R exhibited ratios of NADH- and NADPH-dependent specific activity of 9, 4, 4, 4, and 3.7, respectively, indicating that these mutations can also support cofactor switching of the Slackia KARI.

TABLE 18
Comparison of wild-type S. exigua KARI (Se1_KARI) and double aspartic acid variant.

| | U/mg | | | Kₘ [μM] for cofactor | | k_{cat} [^{s-1}] | | k_{cat}/Kₘ [M⁻¹*s⁻¹] | | |
|---|---|---|---|---|---|---|---|---|---|---|
| Variant (gene) | NADH | NADPH | ratio | NADH | NADPH | NADH | NADPH | NADH | NADPH | ratio |
| Se1_KARI (wt) | 0.15 | 0.32 | 0.47 | 45 ± 10 | <1 | 0.40 | 0.80 | 8,889 | >800,000 | 0.08 |
| Se1_NKR^{S61DS63D} | 0.4 ± 0.05 | 0.042 ± 0.005 | 9.5 | 113 ± 4 | 880 ± 500 | 1 ± 0.13 | 0.1 ± 0.01 | 8,850 ± 1300 | 114 ± 50 | 78 |

### Example 8

### Additional Evolutionary Engineering of Slackia KARI

This example describes how the third generation of Slackia KARI variants were obtained. These variants exhibited improved NADH-dependent activity and abolished NADPH-dependent activity.

In this example, a double NNK library was constructed using SOE PCR. Two fragments were generated using pGV3195^{1AA10} as template and primers 1, 2, 53, and 54 (Table 14) as the respective forward and reverse pairs for fragment generation. Fragments were digested for 1 hr at 37° C. with DpnI, separated on an agarose gel, freeze'n'squeezed (BIORAD) and finally pellet painted (Novagen). The fragments served as templates in assembly PCR using commercial T7 forward and reverse primers as flanking primers. The purified assembly product (Zymo clean up) was restriction digested with NdeI and XhoI, ligated into pET22b(+), and transformed into electro-competent BL21(D3) cells (Lucigen).

Using a high-throughput screening methodology, R58 and S62 were identified as important residues for cofactor switching the Slackia KARI enzyme. The sequencing results derived from this high-throughput screening and the specific activity (in terms of U/mg of purified proteins is summarized in Table 18.

TABLE 18
Summary of Sequencing and Activity Data Compared to Wild-Type
Slackia KARI

| | | | U/mg | | Ratio |
|---|---|---|---|---|---|
| Variant | R58 | S62 | NADH | NADPH | NADH/NADPH |
| Se1_KARI^{his6} | — | — | 0.15 | 0.32 | 0.47 |
| Se1_NKR^{2E8-his6} | P | P | 0.16 | 0.044 | 3.6 |
| Se1_NKR^{3F6-his6} | A | S | 0.086 | 0.03 | 2.9 |
| Se1_NKR^{5C5-his6} | R | E | 0.25 | 0.32 | 0.8 |
| Se1_NKR^{10C2-his6} | P | A | 0.11 | 0.032 | 3.4 |

Mutants 2E8 and 10C2 were further characterized in terms of cofactor K_{M} values and catalytic efficiency. The results are summarized in Table 19. Se1_NKR^{2E8-his6} and Se1_NKR^{10C2-his6} showed a switch in cofactor usage of 46- and 14-fold, respectively. NADPH-dependent activity was virtually abolished. The NADH K_{M} value increased 2-fold compared to wild type, with 87 μM vs. wild type's 45 μM.

TABLE 19
Comparison of S. exigua KARI and Variants Thereof (Generation 3).

| | | Mutations | | | | | | | | | U/mg | | |
|---|---|---|---|---|---|---|---|---|---|---|---|---|---|
| Gen | Variant (gene) | Y35 | L57 | R58 | G60 | S61 | S62 | S63 | I95 | V99 | NADH | NADPH | ratio |
| 0 | Se1_KARI | Y | L | R | G | S | S | S | I | V | 0.15 | 0.32 ± 0.03 | 0.47 |
| 3 | Se1_NKR^{2E8} | | | P | | C | P | D | T | | 0.16 | 0.044 | 3.6 |
| 3 | Se1_NKR^{10C2} | | | p | | C | A | D | T | | 0.11 | 0.032 | 3.4 |
| | | | | | | | Kₘ [μM] for | | | | | | |
| | | | | | | | cofactor | | k_{cat} [^{s-1}] | | k_{cat}/Kₘ [M⁻¹*s⁻¹] | | |
| | | | | | | Gen | NADH | NADPH | NADH | NADPH | NADH | NADPH | ratio |
| | | | | | | 0 | 45 ± 10 | <1 | 0.40 | 0.80 | 8,889 | >800,000 | 0.08 |
| | | | | | | 3 | 87 | >1000 | 0.4 | 0.1 | 4600 | <100 | 46 |
| | | | | | | 3 | 250 | >1000 | 0.27 | 0.08 | 1100 | <80 | 14 |

### Example 9

### Additional Evolutionary Engineering of Slackia KARI

This example describes how the additional generations of Slackia KARI variants were obtained. These variants exhibited improved NADH-dependent activity and abolished NADPH-dependent activity.

In this example, error prone libraries for previous generations with varying concentrations of MnCl₂ (100, 200, 300, and 400 μM final concentrations) were constructed using primers 1 and 2 (Table 14) as the respective forward and reverse primers and Taq polymerase (Roche). The conditions for error prone PCR are illustrated in Table 20. Amplicons were DpnI-digested for 1 hr at 37° C., separated on an agarose gel, freeze'n'squeezed (BIORAD) and finally pellet painted (Novagen). The purified PCR products were restriction digested with NdeI and XhoI, ligated into pET22b(+), and transformed into electro-competent BL21(D3) cells (Lucigen).

TABLE 20
Error Prone PCR Conditions. Total volume of each reaction (rxn) was 100
μL. The volumes given in the table are in terms of μL.

| | 100-μm MnCl₂ | 200-μM MnCl₂ | 300-μM MnCl₂ | 400-μM MnCl₂ |
|---|---|---|---|---|
| | rxn | rxn | rxn | rxn |
| Taq buffer (10x) | 10 | 10 | 10 | 10 |
| dNTPs (25 mM each) | 1 | 1 | 1 | 1 |
| Primer for (10 mM) | 1 | 1 | 1 | 1 |
| Primer rev (10 mM) | 1 | 1 | 1 | 1 |
| Template (100 ng/μL) | 1 | 1 | 1 | 1 |
| MnCl₂ (1 mM) | 10 | 20 | 30 | 40 |
| Taq polymerase | 1.6 | 1.6 | 1.6 | 1.6 |
| PCR_{grade} water | 74.4 | 64.4 | 54.4 | 44.4 |

High-throughput screening methodology revealed that a mutation at position 95, 195V, located in the cofactor binding domain, contributed to reduced NADH K_{M} as compared to the double aspartic acid mutant of Example 8 (Table 21).

TABLE 21
Characterization of S. exigua variant, Gen6, in comparison to the parent, Se1_NKR^{DD-his6}, and the wild-type.

| | |
|---|---|
| Mutations | U/mg |

| Gen | Variant | Y35 | L57 | R58 | G60 | S61 | S62 | S63 | I95 | V99 | NADH | NADPH | ratio |
|---|---|---|---|---|---|---|---|---|---|---|---|---|---|
| 0 | Se1_KARI^{his6} | | | | | | | | | | 0.15 | 0.32 ± 0.03 | 0.47 |
| DD | Se1_NKR^{DD-his6} | | | | | D | | D | | | 0.4 ± 0.05 | 0.04 ± 0.005 | 9.5 |
| 6 | Se1_NKR^{Gen6-his6} | | | | | D | | D | V | | 0.4 ± 0.01 | 0.07 ± 0.0 | 5.7 |
| | | | | | | | Kₘ [μM] for | | | | | | |
| | | | | | | | cofactor | | k_{cat} [^{s-1}] | | k_{cat}/Kₘ [M⁻¹*s⁻¹] | | |
| | | | | | | Gen | NADH | NADPH | NADH | NADPH | NADH | NADPH | ratio |
| | | | | | | 0 | 45 ± 10 | <1 | 0.4 | 0.8 | 8,889 | >800,000 | 0.08 |
| | | | | | | DD | 113 | 880 | 1 | 0.1 | 8850 | 114 | 76 |
| | | | | | | 6 | 47 ± 15 | >1000 | 1 ± 0.01 | 0.18 ± 0.00 | 21000 | 250 | 87 |

The foregoing detailed description has been given for clearness of understanding only and no unnecessary limitations should be understood there from as modifications will be obvious to those skilled in the art.

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth and as follows in the scope of the appended claims.

The disclosures, including the claims, figures and/or drawings, of each and every patent, patent application, and publication cited herein are hereby incorporated herein by reference in their entireties.

1. A recombinant microorganism comprising at least one nucleic acid molecule encoding a ketol-acid reductoisomerase (KARI), wherein said KARI is at least about 60% identical to SEQ ID NO: 2.

2. The recombinant microorganism of claim 1, wherein said KARL is derived from the genus Slackia.

3. The recombinant microorganism of claim 2, wherein said KARI is derived from Slackia exigua.

4. The recombinant microorganism of claim 1, wherein said KARI is encoded by SEQ ID NO: 1.

5-10. (canceled)

11. The recombinant microorganism of claim 1, wherein said KARI is modified to be an NADH-dependent ketol acid reductoisomerase.

12-23. (canceled)

24. A mutant ketol-acid reductoisomerase (KARI) comprising one or more mutations or modifications at positions corresponding to amino acids selected from the group consisting of: (a) tyrosine 35 of the S. exigua KARI (SEQ ID NO: 2); (b) leucine 57 of the S. exigua KARI (SEQ ID NO: 2); (c) arginine 58 of the S. exigua KARI (SEQ ID NO: 2); (d) glycine 60 of the S. exigua KARI (SEQ ID NO: 2); (e) serine 61 of the S. exigua KARI (SEQ ID NO: 2); (f) serine 62 of the S. exigua KARI (SEQ ID NO: 2); (g) serine 63 of the S. exigua KARI (SEQ ID NO: 2); (h) isoleucine 95 of the S. exigua KARI (SEQ ID NO: 2); and (i) valine 99 of the S. exigua KARI (SEQ ID NO: 2).

25. The mutant KARI of claim 24, wherein said tyrosine 35 is replaced with a histidine residue.

26. The mutant KARI of claim 24, wherein said leucine 57 is replaced with a residue selected from serine and arginine.

27. The mutant KARI of claim 24, wherein said arginine 58 is replaced with a residue selected from proline, alanine, or arginine.

28. The mutant KARI of claim 24, wherein said glycine 60 is replaced with a valine residue.

29. The mutant KARI of claim 24, wherein said serine 61 is replaced with a residue selected from aspartic acid, glutamic acid, or cysteine.

30. The mutant KARI of claim 24, wherein said serine 62 is replaced with a residue selected from proline, alanine, or glutamic acid.

31. The mutant KARI of claim 24, wherein said serine 63 is replaced with a residue selected from aspartic acid, glutamic acid, histidine, isoleucine, methionine, arginine, or glutamine.

32. The mutant KARI of claim 24, wherein said isoleucine 95 is replaced with a residue selected from alanine, threonine, valine, and asparagine.

33. The mutant KARI of claim 24, wherein said valine 99 is replaced with a leucine residue.

34. A recombinant microorganism comprising at least one nucleic acid molecule encoding a mutant KARI of claim 24.

35-36. (canceled)

37. The recombinant microorganism of claim 1, wherein said recombinant microorganism further expresses exogenous genes encoding an acetolactate synthase, a dihydroxy acid dehydratase, a keto-isovalerate decarboxylase, and an alcohol dehydrogenase, and wherein said recombinant microorganism produces isobutanol.

38. (canceled)

39. The recombinant microorganism of claim 37, wherein said recombinant microorganism is a yeast microorganism.

40. (canceled)

41. A method of producing isobutanol, comprising:
(a) providing a recombinant microorganism of claim 37; and
(b) cultivating the recombinant microorganism in a culture medium containing a feedstock providing a carbon source, until the isobutanol is produced.

42. A method of producing isobutanol, comprising:
(a) providing a recombinant microorganism of claim 39; and
(b) cultivating the recombinant microorganism in a culture medium containing a feedstock providing a carbon source, until the isobutanol is produced.


SEQUENCE LISTING (88 sequences)

SEQ ID NO: 1 - DNA, 1029 bp, Slackia exigua
gtgtcggtcaagactaaggagaaagaaatggctgtcaccatcttgtacgaacaggacgtcgatcccaaagtcatacagggcctcaaggtcggcatcatcggctacggctcccagggccatgcccatgcgctgaacctcatggattccggcgtcgacgtgcgcgtcggcctgcgcgaaggatcctcttcctggaagaccgccgaagaggccggcctgaaggtcaccgacatggacaccgcggccgaagaagccgacgtcatcatggtcctcgtccccgacgagatccagccgaaggtctaccaggagcacatcgccgcgcacctgaaggcaggcaacacgctcgccttcgcccatggcttcaacatccactacggctacatcgtgccgcccgaggacgtcaacgtcatcatgtgcgctcccaagggcccggggcacatcgtccgccgtcagttcaccgaaggttccggcgtgcccgacctggcctgcgtccagcaggatgccaccggcaacgcctgggatatcgtcctgtcctactgctggggcgtcggcggggcccgttccggcatcatcaaggcgaccttcgccgaggagaccgaggaagacctcttcggcgagcaggccgtgctctgcggaggcctggtggagctggtcaaggccggcttcgagaccctgaccgaggcagggtatccgcccgagctggcatacttcgagtgctatcacgagatgaagatgatcgtcgacctcatgtacgagagcggcatccacttcatgaactactcgatctccaacaccgccgaatacggcgagtactacgccggcccgaaggtcatcaacgagcagtcccgcgaggccatgaaggagatcctgaagcgcattcaggacggctccttcgcccaggaattcgtcgacgactgcaacaacggccataagcgcctgctcgagcagcgcgaagcgatcaatacgcaccccatcgagaccaccggcgcccagatccgcagcatgttctcctggatcaagaaggaagactag

SEQ ID NO: 2 - protein, 342 aa, Slackia exigua
MSVKTKEKEMAVTILYEQDVDPKVIQGLKVGIIGYGSQGHAHALNLMDSGVDVRVGLREGSSSWKTAEEAGLKVTDMDTAAEEADVIMVLVPDEIQPKVYQEHIAAHLKAGNTLAFAHGFNIHYGYIVPPEDVNVIMCAPKGPGHIVRRQFTEGSGVPDLACVQQDATGNAWDIVLSYCWGVGGARSGIIKATFAEETEEDLFGEQAVLCGGLVELVKAGFETLTEAGYPPELAYFECYHEMKMIVDLMYESGIHFMNYSISNTAEYGEYYAGPKVINEQSREAMKEILKRIQDGSFAQEFVDDCNNGHKRLLEQREAINTHPIETTGAQIRSMFSWIKKED

SEQ ID NO: 3 - DNA, 996 bp, Cryptobacterium curtum
atggctgtcaccatcttgcacgaagaggatgcaaatccagcaatcattcaaggaaagaaaattgccattattgggtatggttcacaagggcatgctcatgcgcttaatctgcatgattcaggttgcgatgtacgtgttggcctgcgccgtgattcatcttcttggaagatcgcagaagaggctggtttgaaagttatgaccactgctgaagcggctcgcgaagccaatgtgattatgattttgacacctgatgaaagtcagcgtgcggtgtacgctcaagatattgtcccgcatctgcaaccaggcgatacgcttgcctttgcacatggattcaacatccattttggatacatcgtgccgcccaaggatgttaacgtcatcatggtggcgccaaaaggccctggtcatattgtgcgtcgccaatttactgaaggatccggcgttcctgatcttatctgcgtgcagcaagactttaccgaggatgcctgggatattgccttatcgtatgcgtgggggctcggcggcacgcgcgcgggggttattaagagtacctttaaagacgaaactgaagaggatctctttggcgaacaggcggtgctctgcggcggggttactcacctgatcgaagcaggatacgaagtgctgactgaggctggctatccgggtgagttggcgtattttgaggtgtaccatgaaatgaaaatgattgtggacctcatgtacgaaaacggtatgcatggtatgcgtagctccatttccaatacagctgaatatggtgactattacgctggtccgcggattattaccgaagacaccaaggcagcgatgcgtaccattcttgcccgcattcaagatggatcgtttgcgaaagagtttatcgacgattgcgatcgtggacatgaagtgctgctgagcaagcgtgctgaatatgcgaaccacgaaatcgaacaggttggcgaagaaattcgcaacatgtttagctggatcaaacgatag

SEQ ID NO: 4 - protein, 331 aa, Cryptobacterium curtum
MAVTILHEEDANPAIIQGKKIAIIGYGSQGHAHALNLHDSGCDVRVGLRRDSSSWKIAEEAGLKVMTTAEAAREANVIMILTPDESQRAVYAQDIVPHLQPGDTLAFAHGFNIHFGYIVPPKDVNVIMVAPKGPGHIVRRQFTEGSGVPDLICVQQDFTEDAWDIALSYAWGLGGTRAGVIKSTFKDETEEDLFGEQAVLCGGVTHLIEAGYEVLTEAGYPGELAYFEVYHEMKMIVDLMYENGMHGMRSSISNTAEYGDYYAGPRIITEDTKAAMRTILARIQDGSFAKEFIDDCDRGHEVLLSKRAEYANHEIEQVGEEIRNMFSWIKR

SEQ ID NO: 5 - DNA, 996 bp, Eggerthella sp.
atggctgttacgatctatcacgagaaagacgcgaacccccagctcatccaggacaagaaggtggccatcatcggctacggcagccagggccacgcccatgcgctcaacctgctggactcgggcgtggacgtgcgcgtgggcctgcgcgaggactcgcgctcgcgggccaaggccgaggaggcgggcctcaaggtgatgagcgtcgccgacgctgccgaggaagccgacttcatcatgatcctcacgcccgacgagacccaggccgccacgtacgaagcggacatcgccccgcatctcaaggccggcgacacgctggcgttcgcgcacggcttcaacatccacttcggctacatcgagccgcccgcggacgtcgatgtggtcatgatcgcgccgaagggcccgggccacatggtgcgccgcgtgttcaccgaaggcgcgggcgtgccgtgcctcatctgcgtgaaccaggacgcgtcgggtgcggccaaggatgtggcgctgtcgtacgcgtggggcatcggcggcgcacgcgccggcgtcatcgagaccacgttcaagaacgagaccgaaaccgacctgttcggcgagcaggccgtgctgtgcggcggcgtgacggcgctcatcaacgccggcttcgagacgctggtggaggcgggctatccgcccgagatggcctacttcgagtgcttccacgagatgaagctcatcgtggacctcatgtacgagggcggcatgtcgaacatgcgctactccatctcgaacacggccgagtacggcgactactacgccggcccgcaggtgatcaccgacgagacgaaggccgcgatgaagaccgtgctggcgcgcatccaggacggcagcttcgcccacgagttcatggaggactcgaagaacggccagaagtggctcaaagagcagcgcatggagcacggcaacgcccagatcgaggaagtgggcgccaacatccgctcgatgttcagcttcgtgaggaggtag

SEQ ID NO: 6 - protein, 331 aa, Eggerthella sp.
MAVTIYHEKDANPQLIQDKKVAIIGYGSQGHAHALNLLDSGVDVRVGLREDSRSRAKAEEAGLKVMSVADAAEEADFIMILTPDETQAATYEADIAPHLKAGDTLAFAHGFNIHFGYIEPPADVDVVMIAPKGPGHMVRRVFTEGAGVPCLICVNQDASGAAKDVALSYAWGIGGARAGVIETTFKNETETDLFGEQAVLCGGVTALINAGFETLVEAGYPPEMAYFECFHEMKLIVDLMYEGGMSNMRYSISNTAEYGDYYAGPQVITDETKAAMKTVLARIQDGSFAHEFMEDSKNGQKWLKEQRMEHGNAQIEEVGANIRSMFSFVRR

SEQ ID NO: 7 - DNA, 996 bp, Eggerthella lenta
atggctgttacgatctatcatgagaacgacgcgaacccccagctcatccaggacaagaaagtggccatcatcgggtacggcagccagggccacgcccatgcgctcaacctgaaagactcgggggtcgacgtgcgcgtgggcctgcgcgaggactcgaagtcgcgggtcaaggccgaggaggcgggcctcaaggtgatgagcgtcgccgacgccgccgaggaagccgacttcatcatgatcctcacgcccgacgagacgcaggccgcaacctacgaggccgagatcgccccgcatctcaagcccggcgacaccctcgcgttcgcgcacggcttcaacatccacttcggctacatcacgccgcccgaggacgtggacgtcgtcatgatcgcgccgaagggcccgggccacatggtgcgccgcgtattcaccgagggcgcgggcgtgccgtgtctcatctgcgtccagcaggacgcgtcgggccgggctaaggacgtggcgctgtcctacgcgtggggcatcggcggcgcgcgcgccggcgtcatcgagaccacgttcaagaacgagacggaaaccgacctgttcggcgagcaggccgtgctgtgcggcggcgtgacggccctcatcaacgccgggttcgagacgctcgtcgaggcgggctacccgcccgagatggcctacttcgagtgcttccacgagatgaagctcatcgtggacctcatgtacgaaggcggcatgtcgaacatgcgctactccatctcgaacacggccgagtacggcgactactacgccggcccccaggtgatcaccgacgacgcgaaggccgcgatgaagaccatcctcgagcgcatccaggacggcagcttcgcccacgagttcatggaggactccaagaacggccaggcgtggctgaaggagcagcgcatggagcacggcaacgcccagatcgaggaagtgggcgccagcatccgctcgatgttcagcttcgtgaggaggtag

SEQ ID NO: 8 - protein, 331 aa, Eggerthella lenta
MAVTIYHENDANPQLIQDKKVAIIGYGSQGHAHALNLKDSGVDVRVGLREDSKSRVKAEEAGLKVMSVADAAEEADFIMILTPDETQAATYEAEIAPHLKPGDTLAFAHGFNIHFGYITPPEDVDVVMIAPKGPGHMVRRVFTEGAGVPCLICVQQDASGRAKDVALSYAWGIGGARAGVIETTFKNETETDLFGEQAVLCGGVTALINAGFETLVEAGYPPEMAYFECFHEMKLIVDLMYEGGMSNMRYSISNTAEYGDYYAGPQVITDDAKAAMKTILERIQDGSFAHEFMEDSKNGQAWLKEQRMEHGNAQIEEVGASIRSMFSFVRR

SEQ ID NO: 9 - DNA, 996 bp, Eggerthella sp.
atggctgtcacgatctatcacgagaaagacgcgaacccccagctcatccaggacaagaaggtggccatcatcggctacggcagccagggccacgcccatgcgctcaacctgctggactcgggcgtggacgtgcgcgtgggcctgcgcgaggactcgcgctcgcgggccaaggccgaggaggcgggcctcaaggtgatgagcgtcgccgacgctgccgaggaagccgacttcatcatgatcctcacgcccgacgagacccaggccgccacgtacgaagcggacatcgccccgcatctcaaggccggcgacacgctggcgttcgcgcacggcttcaacatccacttcggctacatcgagtcgcccgcggaggtcgacgtggtcatgatcgcgccgaagggcccgggccacatggtgcgccgcgtgttcaccgaaggcgcgggcgtgccgtgcctcatctgcgtgaaccaggacgcgtcgggccaggccaaggatgtggcgctgtcgtacgcgtggggcatcggcggcgcgcgcgccggcgtcatcgaaaccacgttcaagaacgagaccgaaaccgacctgttcggcgagcaggccgtgctgtgcggcggcgtgacggcgctcatcaacgccggcttcgagacgctggtggaggcgggctatccgcccgagatggcctacttcgagtgcttccacgagatgaagctcatcgtggacctcatgtacgagggcggcatgtcgaacatgcgctactccatctcgaacacggccgagtacggcgactactacgccggcccgcaggtgatcaccgacgagacgaaagccgcgatgaagaccgtcctcgcgcgcatccaggacggcagcttcgcccacgagttcatggaggactcgaagaacggccagaagtggctcaaagagcagcgcatggagcacggcaacgcccagatcgaggaagtgggcgccaacatccgctcgatgttcagcttcgtgaggaggtag

SEQ ID NO: 10 - protein, 331 aa, Eggerthella sp.
MAVTIYHEKDANPQLIQDKKVAIIGYGSQGHAHALNLLDSGVDVRVGLREDSRSRAKAEEAGLKVMSVADAAEEADFIMILTPDETQAATYEADIAPHLKAGDTLAFAHGFNIHFGYIESPAEVDVVMIAPKGPGHMVRRVFTEGAGVPCLICVNQDASGQAKDVALSYAWGIGGARAGVIETTFKNETETDLFGEQAVLCGGVTALINAGFETLVEAGYPPEMAYFECFHEMKLIVDLMYEGGMSNMRYSISNTAEYGDYYAGPQVITDETKAAMKTVLARIQDGSFAHEFMEDSKNGQKWLKEQRMEHGNAQIEEVGANIRSMFSFVRR

SEQ ID NO: 11 - DNA, 1002 bp, Slackia exigua
atggctgtcaccatcttgtacgaacaggacgtcgatcccaaagtcatacagggcctcaaggtcggcatcatcggctacggctcccagggccatgcccatgcgctgaacctcatggattccggcgtcgacgtgcgcgtcggcctgcgcgaaggatcctcttcctggaagaccgccgaagaggccggcctgaaggtcaccgacatggacaccgcggccgaagaagccgacgtcatcatggtcctcgtccccgacgagatccagccgaaggtctaccaggagcacatcgccgcgcacctgaaggcaggcaacacgctcgccttcgcccatggcttcaacatccactacggctacatcgtgccgcccgaggacgtcaacgtcatcatgtgcgctcccaagggcccggggcacatcgtccgccgtcagttcaccgaaggttccggcgtgcccgacctggcctgcgtccagcaggatgccaccggcaacgcctgggatatcgtcctgtcctactgctggggcgtcggcggggcccgttccggcatcatcaaggcgaccttcgccgaggagaccgaggaagacctcttcggcgagcaggccgtgctctgcggaggcctggtggagctggtcaaggccggcttcgagaccctgaccgaggcagggtatccgcccgagctggcatacttcgagtgctatcacgagatgaagatgatcgtcgacctcatgtacgagagcggcatccacttcatgaactactcgatctccaacaccgccgaatacggcgagtactacgccggcccgaaggtcatcaacgagcagtcccgcgaggccatgaaggagatcctgaagcgcattcaggacggctccttcgcccaggaattcgtcgacgactgcaacaacggccataagcgcctgctcgagcagcgcgaagcgatcaatacgcaccccatcgagaccaccggcgcccagatccgcagcatgttctcctggatcaagaaggaagactag

SEQ ID NO: 12 - protein, 333 aa, Slackia exigua
MAVTILYEQDVDPKVIQGLKVGIIGYGSQGHAHALNLMDSGVDVRVGLREGSSSWKTAEEAGLKVTDMDTAAEEADVIMVLVPDEIQPKVYQEHIAAHLKAGNTLAFAHGFNIHYGYIVPPEDVNVIMCAPKGPGHIVRRQFTEGSGVPDLACVQQDATGNAWDIVLSYCWGVGGARSGIIKATFAEETEEDLFGEQAVLCGGLVELVKAGFETLTEAGYPPELAYFECYHEMKMIVDLMYESGIHFMNYSISNTAEYGEYYAGPKVINEQSREAMKEILKRIQDGSFAQEFVDDCNNGHKRLLEQREAINTHPIETTGAQIRSMFSWIKKED

SEQ ID NO: 13 - protein, 7 aa, Artificial Sequence
  acetolactate synthase consensus sequence
SGPGXXN

SEQ ID NO: 14 - protein, 6 aa, Artificial Sequence
  acetolactate synthase consensus sequence
GXXGXX

SEQ ID NO: 15 - protein, 15 aa, Artificial Sequence
  acetolactate synthase consensus sequence
GXXXXGXXXXXAXXX

SEQ ID NO: 16 - protein, 5 aa, Artificial Sequence
  acetolactate synthase consensus sequence
GDXXF

SEQ ID NO: 17 - protein, 9 aa, Artificial Sequence
  dihydroxy acid dehydratase consensus sequence
SLXSRXXIA

SEQ ID NO: 18 - protein, 7 aa, Artificial Sequence
  dihydroxy acid dehydratase consensus sequence
CDKXXPG

SEQ ID NO: 19 - protein, 10 aa, Artificial Sequence
  dihydroxy acid dehydratase consensus sequence
GXCXGXXTAN

SEQ ID NO: 20 - protein, 5 aa, Artificial Sequence
  dihydroxy acid dehydratase consensus sequence
GGSTN

SEQ ID NO: 21 - protein, 11 aa, Artificial Sequence
  dihydroxy acid dehydratase consensus sequence
GPXGXPGMRXE

SEQ ID NO: 22 - protein, 10 aa, Artificial Sequence
  dihydroxy acid dehydratase consensus sequence
ALXTDGRXSG

SEQ ID NO: 23 - protein, 7 aa, Artificial Sequence
  dihydroxy acid dehydratase consensus sequence
GHXXPEA

SEQ ID NO: 24 - protein, 7 aa, Artificial Sequence
  2-keto-acid decarboxylase consensus sequence
FGXXGXX

SEQ ID NO: 25 - protein, 9 aa, Artificial Sequence
  2-keto-acid decarboxylase consensus sequence
XTXGXGXXX

SEQ ID NO: 26 - protein, 9 aa, Artificial Sequence
  2-keto-acid decarboxylase consensus sequence
NXXAGXXAE

SEQ ID NO: 27 - protein, 6 aa, Artificial Sequence
  2-keto-acid decarboxylase consensus sequence
XXXIXG

SEQ ID NO: 28 - protein, 8 aa, Artificial Sequence
  2-keto-acid decarboxylase consensus sequence
GDGXXQXT

SEQ ID NO: 29 - protein, 6 aa, Artificial Sequence
  alcohol dehydrogenase consensus sequence
CXXDXH

SEQ ID NO: 30 - protein, 8 aa, Artificial Sequence
  alcohol dehydrogenase consensus sequence
GHEXXGXV

SEQ ID NO: 31 - protein, 7 aa, Artificial Sequence
  alcohol dehydrogenase consensus sequence
XXXGXXX

SEQ ID NO: 32 - protein, 7 aa, Artificial Sequence
  alcohol dehydrogenase consensus sequence
CXXCXXC

SEQ ID NO: 33 - protein, 6 aa, Artificial Sequence
  alcohol dehydrogenase consensus sequence
XXXXTX

SEQ ID NO: 34 - protein, 6 aa, Artificial Sequence
  alcohol dehydrogenase consensus sequence
GXGXXG

SEQ ID NO: 35 - DNA, 20 bp, Artificial Sequence
  Primer T7_for
taatacgactcactataggg

SEQ ID NO: 36 - DNA, 19 bp, Artificial Sequence
  Primer T7_rev
gctagttattgctcagcgg

SEQ ID NO: 37 - DNA, 39 bp, Artificial Sequence
  Primer Se_I33NNK_for
ggtcttaaagttggtatcnnkggttacggttcccaaggt

SEQ ID NO: 38 - DNA, 39 bp, Artificial Sequence
  Primer Se_I33NNK_rev
accttgggaaccgtaaccmnngataccaactttaagacc

SEQ ID NO: 39 - DNA, 33 bp, Artificial Sequence
  Primer Se_G34NNK_for
aaagttggtatcatcnnktacggttcccaaggt

SEQ ID NO: 40 - DNA, 33 bp, Artificial Sequence
  Primer Se_G34NNK_rev
accttgggaaccgtamnngatgataccaacttt

SEQ ID NO: 41 - DNA, 33 bp, Artificial Sequence
  Primer Se_Y35NNK_for
gttggtatcatcggtnnkggttcccaaggtcac

SEQ ID NO: 42 - DNA, 33 bp, Artificial Sequence
  Primer Se_Y35NNK_rev
gtgaccttgggaaccmnnaccgatgataccaac

SEQ ID NO: 43 - DNA, 33 bp, Artificial Sequence
  Primer Se_G36NNK_for
ggtatcatcggttacnnktcccaaggtcacgct

SEQ ID NO: 44 - DNA, 33 bp, Artificial Sequence
  Primer Se_G36NNK_rev
agcgtgaccttgggamnngtaaccgatgatacc

SEQ ID NO: 45 - DNA, 33 bp, Artificial Sequence
  Primer Se_L57NNK_for
gatgttagagttggcnnkagagaaggctcatct

SEQ ID NO: 46 - DNA, 33 bp, Artificial Sequence
  Primer Se_L57NNK_rev
agatgagccttctctmnngccaactctaacatc

SEQ ID NO: 47 - DNA, 33 bp, Artificial Sequence
  Primer Se_R58NNK_for
gttagagttggcttannkgaaggctcatctagt

SEQ ID NO: 48 - DNA, 33 bp, Artificial Sequence
  Primer Se_R58NNK_rev
actagatgagccttcmnntaagccaactctaac

SEQ ID NO: 49 - DNA, 33 bp, Artificial Sequence
  Primer Se_G60NNK_for
gttggcttaagagaannktcatctagttggaaa

SEQ ID NO: 50 - DNA, 33 bp, Artificial Sequence
  Primer Se_G60NNK_rev
tttccaactagatgamnnttctcttaagccaac

SEQ ID NO: 51 - DNA, 33 bp, Artificial Sequence
  Primer Se_S61NNK_for
ggcttaagagaaggcnnktctagttggaaaacg

SEQ ID NO: 52 - DNA, 33 bp, Artificial Sequence
  Primer Se_S61NNK_rev
cgttttccaactagamnngccttctcttaagcc

SEQ ID NO: 53 - DNA, 33 bp, Artificial Sequence
  Primer Se_S62NNK_for
ttaagagaaggctcannkagttggaaaacggct

SEQ ID NO: 54 - DNA, 33 bp, Artificial Sequence
  Primer Se_S62NNK_rev
agccgttttccaactmnntgagccttctcttaa

SEQ ID NO: 55 - DNA, 33 bp, Artificial Sequence
  Primer Se_S63NNK_for
agagaaggctcatctnnktggaaaacggctgag

SEQ ID NO: 56 - DNA, 33 bp, Artificial Sequence
  Primer Se_S63NNK_rev
ctcagccgttttccamnnagatgagccttctct

SEQ ID NO: 57 - DNA, 33 bp, Artificial Sequence
  Primer Se_L90NNK_for
gatgtcatcatggttnnkgtgcctgatgaaatt

SEQ ID NO: 58 - DNA, 33 bp, Artificial Sequence
  Primer Se_L90NNK_rev
aatttcatcaggcacmnnaaccatgatgacatc

SEQ ID NO: 59 - DNA, 33 bp, Artificial Sequence
  Primer Se_I95NNK_for
ttggtgcctgatgaannkcaacctaaggtatat

SEQ ID NO: 60 - DNA, 33 bp, Artificial Sequence
  Primer Se_I95NNK_rev
atataccttaggttgmnnttcatcaggcaccaa

SEQ ID NO: 61 - DNA, 33 bp, Artificial Sequence
  Primer Se_V99NNK_for
gaaattcaacctaagnnktatcaggaacatatc

SEQ ID NO: 62 - DNA, 33 bp, Artificial Sequence
  Primer Se_V99NNK_rev
gatatgttcctgatamnncttaggttgaatttc

SEQ ID NO: 63 - DNA, 27 bp, Artificial Sequence
  Primer Se_recomb1_Y35YAC_for
ggtatcatcggtyacggttcccaaggt

SEQ ID NO: 64 - DNA, 27 bp, Artificial Sequence
  Primer Se_recomb1_Y35YAC_rev
accttgggaaccgtraccgatgatacc

SEQ ID NO: 65 - DNA, 36 bp, Artificial Sequence
  Primer Se_recomb2a_for
ggcttaagagaagkatsctctagttggaaaacggct

SEQ ID NO: 66 - DNA, 36 bp, Artificial Sequence
  Primer Se_recomb2b_for
ggcttaagagaagkatsctctgattggaaaacggct

SEQ ID NO: 67 - DNA, 36 bp, Artificial Sequence
  Primer Se_recomb2c_for
ggcttaagagaagkatsctctcagtggaaaacggct

SEQ ID NO: 68 - DNA, 36 bp, Artificial Sequence
  Primer Se_recomb2a_rev
agccgttttccaactagagsatmcttctcttaagcc

SEQ ID NO: 69 - DNA, 36 bp, Artificial Sequence
  Primer Se_recomb2b_rev
agccgttttccaatcagagsatmcttctcttaagcc

SEQ ID NO: 70 - DNA, 36 bp, Artificial Sequence
  Primer Se_recomb2c_rev
agccgttttccactgagagsatmcttctcttaagcc

SEQ ID NO: 71 - DNA, 33 bp, Artificial Sequence
  Primer Se_recomb3a_for
cctgatgaaahccaacctaagktatatcaggaa

SEQ ID NO: 72 - DNA, 33 bp, Artificial Sequence
  Primer Se_recomb3b_for
cctgatgaagyacaacctaagktatatcaggaa

SEQ ID NO: 73 - DNA, 33 bp, Artificial Sequence
  Primer Se_recomb3a_rev
ttcctgatatamcttaggttggdtttcatcagg

SEQ ID NO: 74 - DNA, 33 bp, Artificial Sequence
  Primer Se_recomb3b_rev
ttcctgatatamcttaggttgtrcttcatcagg

SEQ ID NO: 75 - DNA, 27 bp, Artificial Sequence
  Primer Se_recomb4a_L57TYA_for
gtcgatgttagagttggctyaagagaa

SEQ ID NO: 76 - DNA, 27 bp, Artificial Sequence
  Primer Se_recomb4a_L57TYA_rev
ttctcttragccaactctaacatcgac

SEQ ID NO: 77 - DNA, 27 bp, Artificial Sequence
  Primer Se_recomb4b_L57CRA_for
gtcgatgttagagttggccraagagaa

SEQ ID NO: 78 - DNA, 27 bp, Artificial Sequence
  Primer Se_recomb4b_L57CRA_rev
ttctcttyggccaactctaacatcgac

SEQ ID NO: 79 - DNA, 33 bp, Artificial Sequence
  Primer Se_L57NNK_R58P_S61TSC_for
agagttggcnnkccagaaggctsctctagttgg

SEQ ID NO: 80 - DNA, 33 bp, Artificial Sequence
  Primer Se_L57NNK_R58P_S61TSC_rev
ccaactagagsagccttctggmnngccaactct

SEQ ID NO: 81 - DNA, 39 bp, Artificial Sequence
  Primer Serec4_fancy3_rec_A_for
gttagagttggcgtaccagaagkatgctctagttggaaa

SEQ ID NO: 82 - DNA, 39 bp, Artificial Sequence
  Primer Serec4_fancy3_rec_A_rev
tttccaactagagcatmcttctggtacgccaactctaac

SEQ ID NO: 83 - DNA, 36 bp, Artificial Sequence
  Primer Serec4_fancy3_rec_B_for
cctgatgaaryacaacctaagktatatcaggaacat

SEQ ID NO: 84 - DNA, 36 bp, Artificial Sequence
  Primer Serec4_fancy3_rec_B_rev
atgttcctgatatamcttaggttgtryttcatcagg

SEQ ID NO: 85 - DNA, 39 bp, Artificial Sequence
  Primer Se1_S63D_S61D_for
ggcttaagagaaggcgactctgactggaaaacggctgag

SEQ ID NO: 86 - DNA, 39 bp, Artificial Sequence
  Primer Se1_S63D_S61D_rev
ctcagccgttttccagtcagagtcgccttctcttaagcc

SEQ ID NO: 87 - DNA, 39 bp, Artificial Sequence
  Primer SeAA10R58S62NNK_for
gttggcttannkgaaggctgcnnkgattggaaaacggct

SEQ ID NO: 88 - DNA, 39 bp, Artificial Sequence
  Primer SeAA10R58S62NNK_rev
agccgttttccaatcmnngcagccttcmnntaagccaac